(19) United States
(12) Patent Application Publication
(10) Pub. No.: US 2026/0071228 A1
(43) Pub. Date: Mar. 12, 2026

(54) GUIDE POLYNUCLEOTIDE MULTIPLEXING

(71) Applicant: PIONEER HI-BRED INTERNATIONAL, INC., JOHNSTON, IA (US)

(72) Inventors: WILLIAM JAMES GORDON-KAMM, URBANDALE, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 19/108,376

(22) PCT Filed: Aug. 9, 2023
(86) PCT No.: PCT/US2023/071900

§ 371 Date: Mar. 3, 2025

Related U.S. Application Data
(60) Provisional application No. 63/370,819, filed on Aug. 9, 2022.
(60) Provisional application No. 63/380,781, filed on Oct. 25, 2022.

(51) Int. Cl. C12N 15/82; C12N 9/22; C12N 15/11

(52) U.S. Cl. CPC C12N 15/8207; C12N 9/22; C12N 9/226; C12N 15/11; C12Y 301/26003; C12N 2310/20; C12N 2310/531

(57) ABSTRACT

The present disclosure relates to methods and compositions for expressing multiple guide polynucleotides from one or more transcripts. Compositions and methods for delivering a plurality of guide polynucleotides to target multiple independent sites in a cell's genome are also provided.

### FIELD OF THE DISCLOSURE

The disclosure relates to the field of plant molecular biology, in particular, to compositions and methods for delivering multiplexed guide polynucleotides to a cell.

### BACKGROUND

Polygenic plant traits such as yield involve the coordinated effect of tens, hundreds, or even thousands of genes. To improve these traits using CRISPR-based genome editing, approaches that enable the simultaneously delivery of tens, hundreds, or even thousands of guide polynucleotides are needed.

The present disclosure relates to molecular strategies for expressing multiple guide polynucleotides from a single transcript that is processed to produce multiple, individual guide polynucleotides capable of directing one or more Cas polypeptides to a DNA target site in a plant cell.

### SUMMARY OF DISCLOSURE

Provided herein are methods and compositions for expressing multiple guide polynucleotides from a single transcript.

In a first aspect, the disclosure provides a method of delivering a plurality of guide polynucleotides for multiplexed genome editing of multiple genomic targets in a plant cell, the method comprising: providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop.

In some aspects of the method, the at least one stem loop is a rabbit-ear stem loop. Generally, Y-shaped RNA dimers (loops) with a longer extension (stem) are referred to as rabbit-ear stem loop based on the structural characteristics of the shape of a rabbit ear or the letter Y.

In some aspects of the method, the eukaryotic RNase III is endogenous to the plant cell.

In some aspects of the method, the eukaryotic RNase III is maize-optimized yeast RNase III.

In some aspects of the method, the eukaryotic RNase III is a heterologous RNase III and the method further comprises: engineering the heterologous RNase III to comprise a polypeptide domain capable of recognizing the stem loop of the RNA molecule.

In some aspects of the method, the eukaryotic RNase III is a heterologous RNase III and the method further comprises: engineering the heterologous RNase III as a heterodimer molecule comprising two distinct polypeptide domains capable of recognizing two stem loops of the RNA molecule. In some aspects, the two distinct polypeptide domains are operatively-associated through a linker. In some aspects, the heterologous RNase III exhibits increased specificity to double-stranded RNA molecules such that a proportionately higher amount of the RNA molecule comprising the at least two guide polynucleotide sequences are cleaved compared to a control.

In another aspect, the disclosure provides a composition for delivering a plurality of guide polynucleotides for multiplexed genome editing of multiple genomic targets in a plant cell, the composition comprising: (a) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and (b) a heterologous eukaryotic RNase III that cleaves an RNA molecule transcribed from the polynucleotide molecule, wherein the RNA molecule having the RNase III recognition sequence comprises at least one stem loop.

In some aspects of the composition, the heterologous eukaryotic RNase III comprises a polypeptide domain that recognizes the stem loop of the RNA molecule.

In some aspects of the composition, the heterologous eukaryotic RNase III is a heterodimer molecule comprising two distinct polypeptide domains that recognize two stem loops of the RNA molecule. In some aspects, the two distinct polypeptide domains are operatively-associated through a linker.

In another aspect, the disclosure provides a plant cell or cells comprising provides a composition for delivering a plurality of guide polynucleotides for multiplexed genome editing of multiple genomic targets in a plant cell, the composition comprising: (a) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and (b) a heterologous eukaryotic RNase III that cleaves an RNA molecule transcribed from the polynucleotide molecule, wherein the RNA molecule having the RNase III recognition sequence comprises at least one stem loop. In some aspects, the heterologous eukaryotic RNase III comprises a polypeptide domain that recognizes the stem loop of the RNA molecule. In some aspects, the heterologous eukaryotic RNase III is a heterodimer molecule comprising two distinct polypeptide domains that recognize two stem loops of the RNA molecule. In some aspects, the two distinct polypeptide domains are operatively-associated through a linker.

In yet another aspect, the disclosure provides a method for editing a plant genome, the method comprising: providing a plant cell with: (a) a Cas endonuclease; and (b) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects of the method, the first and/or second site-specific modification in the first target nucleotide sequence is an insertion, a deletion, a substitution, a transversion, and/or a transition.

In some aspects of the method, the method further comprises providing a donor DNA to the plant cell.

In some aspects of the method, the Cas endonuclease is a Cas12 endonuclease or a Cas9 endonuclease.

In some aspects of the method, the Cas endonuclease comprises a deactivated Cas endonuclease (dCas) complexed to a deaminase. In some aspects, the deactivated Cas endonuclease is dCas12f or dCas9. In some aspects, the deaminase is a cytosine deaminase or an adenosine deaminase.

In some aspects of the method, the Cas endonuclease comprises a deactivated Cas endonuclease (dCas) complexed to a reverse transcriptase. In some aspects, the deactivated Cas endonuclease is dCas12f or dCas9.

In some aspects of the method, the Cas endonuclease has nickase activity.

In some aspects of the method, the at least one stem loop is a rabbit-ear stem loop.

In some aspects of the method, the eukaryotic RNase III is endogenous to the plant cell.

In some aspects of the method, the eukaryotic RNase III is a heterologous RNase III and the method further comprises: engineering the heterologous RNase III to comprise a polypeptide domain capable of recognizing the stem loop of the RNA molecule.

In some aspects of the method, the RNase III is a heterologous RNase III and the method further comprises: engineering the heterologous RNase III as a heterodimer molecule comprising two distinct polypeptide domains capable of recognizing two stem loops of the RNA molecule. In some aspects, the two distinct polypeptide domains are operatively-associated through a linker.

In a further aspect, the disclosure provides a method of delivering a plurality of guide polynucleotides for multiplexed genome editing of multiple genomic targets in a plant cell, the method comprising: providing a polynucleotide molecule to the plant cell, the polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In some aspects of the method, a ribozyme encoded by the self-cleaving ribozyme-encoding nucleotide sequence is a Hammer-head self-cleaving ribozyme. Generally, the hammerhead ribozyme refers to a catalytic RNA motif that catalyzes reversible cleavage/ligation reactions at a specific site within an RNA molecule. The term Hammer-head generally refers to certain secondary structure patterns that resemble a hammerhead shark.

In yet a further aspect, the disclosure provides a composition for delivering a plurality of guide polynucleotides for multiplexed genome editing of multiple genomic targets in a plant cell, the composition comprising a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotides is flanked by the RNase recognition sequence at a 5′ end.

In another aspect, the disclosure provides a plant cell or cells comprising a composition comprising a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotides is flanked by the RNase recognition sequence at a 5′ end.

In another aspect, the disclosure provides a method for editing a plant genome, the method comprising: providing a plant cell with: (a) a Cas endonuclease; and (b) a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end, expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects of the method, the first and/or second site-specific modification in the first target nucleotide sequence is an insertion, a deletion, a substitution, a transversion, and/or a transition. In some aspects, the method further comprises providing a donor DNA to the plant cell.

In some aspects of the method, the Cas endonuclease is a Cas12 endonuclease or a Cas9 endonuclease.

In some aspects of the method, the Cas endonuclease comprises a deactivated Cas endonuclease (dCas) complexed to a deaminase. In some aspects, the deactivated Cas endonuclease is dCas12f or dCas9. In some aspects, the deaminase is a cytosine deaminase or an adenosine deaminase.

In some aspects of the method, the Cas endonuclease comprises a deactivated Cas endonuclease (dCas) complexed to a reverse transcriptase. In some aspects, the deactivated Cas endonuclease is dCas12f or dCas9.

In some aspects of the method, the Cas endonuclease has nickase activity.

In some aspects of the method, a ribozyme encoded by the self-cleaving ribozyme-encoding nucleotide sequence is a Hammer-head self-cleaving ribozyme.

In another aspect, the disclosure provides a method of delivering a plurality of guide polynucleotides for multiplexed genome editing of multiple genomic targets in a plant cell, the method comprising: providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop.

In some aspects of the method, the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

In some aspects of the method, the ribonuclease domain of the viral coat protein comprises a PilT N-terminus (PIN) ribonuclease domain. In some aspects, the PIN ribonuclease domain is from a human telomerase-binding protein EST1A.

In some aspects of the method, the viral coat protein is a MS2 bacteriophage coat protein. In some aspects, the method further comprises: engineering the MS2 bacteriophage coat protein to comprise a polypeptide domain capable of recognizing the stem loop of the RNA molecule. In some aspects, the MS2 bacteriophage coat protein is a homodimer molecule comprising two polypeptide domains capable of recognizing two stem loops of the RNA molecule.

In some aspects of the method, the 5′ and 3′ ends of the polynucleotide molecule comprising at least two guide polynucleotide sequences are flanked with polynucleotide sequences encoding stem loop-forming RNA.

In some aspects of the method, the viral coat protein is a PP7 bacteriophage coat protein. In some aspects, the method further comprises engineering the PP7 bacteriophage coat protein to comprise a polypeptide domain capable of recognizing the stem loop of the RNA molecule.

In some aspects of the method, the 5′ end of the polynucleotide molecule comprising at least two guide polynucleotide sequences is flanked with a polynucleotide sequence encoding stem loop-forming RNA and the 3′ end of the polynucleotide molecule comprising at least two guide polynucleotide sequences is flanked with a polynucleotide sequence encoding a ribozyme.

In yet another aspect, the disclosure provides a composition for delivering a plurality of guide polynucleotides for multiplexed genome editing of multiple genomic targets in a plant cell, the composition comprising: (a) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and (b) a double-stranded RNA-specific protein comprising a ribonuclease domain that cleaves an RNA molecule transcribed from the polynucleotide molecule, wherein the RNA molecule having the double-stranded RNA-specific protein recognition sequence comprises at least one stem loop.

In some aspects of the composition, the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

In some aspects of the composition, the ribonuclease domain of the viral coat protein comprises a PilT N-terminus (PIN) ribonuclease domain. In some aspects, the PIN ribonuclease domain is from a human telomerase-binding protein EST1A.

In some aspects of the composition, the viral coat protein is a MS2 bacteriophage coat protein. In some aspects, the MS2 bacteriophage coat protein comprises a polypeptide domain capable of recognizing the stem loop of the RNA molecule. In some aspects, the MS2 bacteriophage coat protein is a homodimer molecule comprising two polypeptide domains capable of recognizing two stem loops of the RNA molecule.

In some aspects of the composition, the viral coat protein is a PP7 bacteriophage coat protein. In some aspects, the PP7 bacteriophage coat protein comprises a polypeptide domain capable of recognizing the stem loop of the RNA molecule.

In another aspects, the disclosure provides a plant cell or cells comprising a composition for delivering a plurality of guide polynucleotides for multiplexed genome editing of multiple genomic targets in a plant cell, the composition comprising: (a) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and (b) a double-stranded RNA-specific protein comprising a ribonuclease domain that cleaves an RNA molecule transcribed from the polynucleotide molecule, wherein the RNA molecule having the double-stranded RNA-specific protein recognition sequence comprises at least one stem loop.

In another aspect, the disclosure provides a method for editing a plant genome, the method comprising: providing a plant cell with: (a) a Cas endonuclease; and (b) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects of the method, the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

In some aspects of the method, the first and/or second site-specific modification in the second target nucleotide sequence is an insertion, a deletion, a substitution, a transversion, and/or a transition. In some aspects, the method further comprises providing a donor DNA to the plant cell.

In some aspects of the method, the Cas endonuclease is a Cas12 endonuclease or a Cas9 endonuclease.

In some aspects of the method, the Cas endonuclease comprises a deactivated Cas endonuclease (dCas) complexed to a deaminase. In some aspects, the deactivated Cas endonuclease is dCas12f or dCas9. In some aspects, the deaminase is a cytosine deaminase or an adenosine deaminase.

In some aspects of the method, the Cas endonuclease comprises a deactivated Cas endonuclease (dCas) complexed to a reverse transcriptase. In some aspects, the deactivated Cas endonuclease is dCas12f or dCas9.

In some aspects of the method, the Cas endonuclease has nickase activity.

In some aspects of the method, the ribonuclease domain of the viral coat protein comprises a PilT N-terminus (PIN) ribonuclease domain. In some aspects, the PIN ribonuclease domain is from a human telomerase-binding protein EST1A.

In some aspects of the method, the viral coat protein is a MS2 bacteriophage coat protein. In some aspects, the method further comprises engineering the MS2 bacteriophage coat protein to comprise a polypeptide domain capable of recognizing the stem loop of the RNA molecule. In some aspects, the MS2 bacteriophage coat protein is a homodimer molecule comprising two polypeptide domains capable of recognizing two stem loops of the RNA molecule.

In some aspects of the method, the viral coat protein is a PP7 bacteriophage coat protein. In some aspects, the method further comprises engineering the PP7 bacteriophage coat protein to comprise a polypeptide domain capable of recognizing the stem loop of the RNA molecule.

In some aspects of the method, the 5′ and 3′ ends of the polynucleotide molecule comprising the at least two guide polynucleotide sequences are flanked with polynucleotide sequences encoding stem loop-forming RNA.

In some aspects of the method, the 5′ end of the polynucleotide molecule comprising the at least two guide polynucleotide sequences is flanked with a polynucleotide sequence encoding stem loop-forming RNA and the 3′ end of the polynucleotide molecule comprising the at least two guide polynucleotide sequences is flanked with a polynucleotide sequence encoding a ribozyme.

In another aspect, the disclosure provides a method of delivering a plurality of guide polynucleotides for multiplexed genome editing of multiple genomic targets in a plant cell, the method comprising: providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by an RNase Z recognition sequence; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In yet another aspect, the disclosure provides a composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by an RNase recognition sequence.

In another aspect, the disclosure provides a plant cell or cells comprising a composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by an RNase recognition sequence.

In a further aspect, the disclosure provides a method for editing a plant genome, the method comprising: providing a plant cell with: (a) a Cas endonuclease; and (b) a polynucleotide molecule comprising at least two guide polynucleotide sequences targeting at least two distinct genomic target sites in the plant cell, wherein each of the at least two guide polynucleotide sequences is flanked by an RNase Z recognition sequence, expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects of the method, the first and/or second site-specific modification in the second target nucleotide sequence is an insertion, a deletion, a substitution, a transversion, and/or a transition. In some aspects, the method further comprises providing a donor DNA to the plant cell.

In some aspects of the method, the Cas endonuclease is a Cas12 endonuclease or a Cas9 endonuclease.

In some aspects of the method, the Cas endonuclease comprises a deactivated Cas endonuclease (dCas) complexed to a deaminase. In some aspects, the deactivated Cas endonuclease is dCas12f or dCas9. In some aspects, the deaminase is a cytosine deaminase or an adenosine deaminase.

In some aspects of the method, the Cas endonuclease comprises a deactivated Cas endonuclease (dCas) complexed to a reverse transcriptase. In some aspects, the deactivated Cas endonuclease is dCas12f or dCas9.

In some aspects of the method, the Cas endonuclease has nickase activity.

In another aspect, the disclosure provides a method of delivering a plurality of guide polynucleotides for multiplexed genome editing of multiple genomic targets in a plant cell, the method comprising: providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In some aspects, a ribozyme encoded by each of the self-cleaving ribozyme-encoding nucleotide sequences is a Hammer-head self-cleaving ribozyme.

In another aspect, the disclosure provides a composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence.

In another aspect, the disclosure provides a plant cell or cells comprising a composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence.

In a further aspect, the disclosure provides a method for editing a plant genome, the method comprising: providing a plant cell with: (a) a Cas endonuclease; and (b) a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence, expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects of the method, the first and/or second site-specific modification in the second target nucleotide sequence is an insertion, a deletion, a substitution, a transversion, and/or a transition. In some aspects, the method further comprises providing a donor DNA to the plant cell.

In some aspects of the method, the Cas endonuclease is a Cas12 endonuclease or a Cas9 endonuclease.

In some aspects of the method, the Cas endonuclease comprises a deactivated Cas endonuclease (dCas) complexed to a deaminase. In some aspects, the deactivated Cas endonuclease is dCas12f or dCas9. In some aspects, the deaminase is a cytosine deaminase or an adenosine deaminase.

In some aspects of the method, the Cas endonuclease comprises a deactivated Cas endonuclease (dCas) complexed to a reverse transcriptase. In some aspects, the deactivated Cas endonuclease is dCas12f or dCas9.

In some aspects of the method, the Cas endonuclease has nickase activity.

In some aspects of the method, a ribozyme encoded by the self-cleaving ribozyme-encoding nucleotide sequence is a Hammer-head self-cleaving ribozyme.

In another aspect, the disclosure provides, a method for generating a plurality of guide RNA molecules for genetic modification in a plant cell, the method comprising providing to the plant cell a polynucleotide expression cassette encoding two or more guide RNA sequences separated by one or more tRNA cleavage sequence, wherein each guide RNA sequence comprises a 3′ spacer sequence that exhibits complementarity to a target sequence in the plant cell, wherein the plant cell's tRNA processing system cleaves a transcript generated from the transcribed polynucleotide expression cassette, thereby generating the plurality of guide RNA sequences.

In some aspects of the method, the guide RNA sequences target multiple sites in one or more chromosome of the plant cell.

In some aspects of the method, the guide RNA sequences target one or more multigene families in the plant cell.

In some aspects of the method, the genetic modification is RNA guided chromosomal genome modification in the presence of a CRISPR-associated polypeptide.

In some aspects of the method, the genetic modification is a chromosomal genome modification selected from the group consisting of targeted mutation, homology-dependent repair, homology directed recombination, transcriptional activation, transcriptional downregulation, insertion, deletion, epigenome modification, and a combination of the foregoing.

In some aspects of the method, the genetic modification is RNA guided base editing.

In some aspects of the method, the tRNA cleavage sequence includes a pretRNA acceptor stem, a D-loop arm and a TY′C-loop arm.

In some aspects of the method, the tRNA cleavage sequence includes an active site for one or more of RNase P and/or RNase Z and/or RNase E.

In yet another aspect, the disclosure provides a nucleic acid expression cassette for generating a plurality of guide RNA molecules for genetic modification in a plant cell comprising a polynucleotide encoding two or more guide RNA sequences separated by one or more tRNA cleavage sequence, wherein each guide RNA sequence comprises a 3′ spacer sequence that exhibits complementarity to a target sequence in the plant cell.

In some aspects of expression cassette, the tRNA cleavage sequence includes a pretRNA acceptor stem, a D-loop arm and a TY′C-loop arm.

In some aspects of the expression cassette, the tRNA cleavage sequence includes an active site for one or more of RNase P and/or RNase Z and/or RNase E.

In some aspects of the expression cassette, the nucleic acid expression cassette includes a guide RNA-tRNA-guide RNA configuration such that the spacer sequence is at the 3′ position.

### BRIEF DESCRIPTION OF THE DRAWINGS

The disclosure can be more fully understood from the following detailed description and the accompanying drawings and Sequence Listing, which form a part of this application.

FIG. 1 illustrates a maize-optimized Cas endonuclease expression cassette. I, II, III, IV, V, and VI correspond to a Zea mays Ubiquitin promoter (SEQ ID NO:8), Zea mays Ubiquitin 5′ UTR (SEQ ID NO:9), Zea mays Ubiquitin intron 1 (SEQ ID NO:10), Zea mays optimized gene encoding a SpaCas12f1 engineered variant containing a ST-LS1 Intron 2 (SEQ ID NOs: 11-13), Zea mays optimized sequence encoding SV40 NLS (SEQ ID NO:14), and Zea mays Ubiquitin terminator (SEQ ID NO: 15), respectively.

FIGS. 2A-2E illustrate a method for excising one or more guide RNAs from a primary transcript. I corresponds to a Zea mays U6 promoter (SEQ ID NO:16), II to a DNA encoding a RNase Z recognition site (SEQ ID NO:17-18), III to a DNA encoding a gRNA Cas Recognition domain for SpCas12f1 (SEQ ID NO:19), IV to a DNA encoding a SpCas12f1 gRNA Variable Targeting domain (SEQ ID NO:20-24), V to a DNA encoding a HDV ribozyme (SEQ ID NO:25), VI to a Zea mays U6 terminator (SEQ ID NO:26), VII to a Zea mays Ubiquitin promoter (SEQ ID NO: 8), VIII to a Zea mays Ubiquitin 5′ UTR (SEQ ID NO:9), IX to a Zea mays Ubiquitin intron 1 (SEQ ID NO:10), and X to a Zea mays Ubiquitin terminator (SEQ ID NO:15). FIG. 2B illustrates a polymerase III DNA expression cassette comprising a DNA sequence encoding a guide RNA flanked by a DNA sequence encoding an RNase Z recognition site and a DNA sequence encoding a ribozyme. FIG. 2C illustrates a polymerase II DNA expression cassette comprising a DNA sequence encoding a guide RNA flanked by a DNA sequence encoding an RNase Z recognition site and a DNA sequence encoding a ribozyme. FIG. 2A illustrates excision of a guide RNA from a primary transcript via RNase Z (scissors represent an RNase Z cleavage site) and the self-cleaving ribozyme (triangle represents the ribozyme self-cleavage site). FIG. 2E illustrates a polymerase III DNA expression cassette comprising a first DNA sequence encoding a first guide RNA and a second DNA sequence encoding a second guide RNA, wherein each DNA sequence encoding a guide RNA is flanked by a DNA sequence encoding an RNase Z recognition site and a DNA sequence encoding a ribozyme. FIG. 2D illustrates excision of a first and a second guide RNA from a primary transcript via RNase Z (scissors represent an RNase Z cleavage site) and the self-cleaving ribozyme (triangles represent the ribozyme self-cleavage site).

FIGS. 3A and 3B illustrate another method for excising one or more guide RNAs from a primary transcript. I corresponds to a Zea mays U6 promoter (SEQ ID NO:16), III to a DNA encoding a gRNA Cas Recognition domain for SpCas12f1 (SEQ ID NO:19), IV to a DNA encoding a SpCas12f1 gRNA Variable Targeting domain (SEQ ID NO:20-24), V to a DNA encoding a HDV ribozyme (SEQ ID NO:25), and VI to a Zea mays U6 terminator (SEQ ID NO: 26). FIG. 3B illustrates a DNA expression cassette comprising a DNA sequence encoding a guide RNA flanked by a 3′ DNA sequence encoding a ribozyme. FIG. 3A illustrates excision of a guide RNA from a primary transcript via the self-cleaving ribozyme (triangle represents the ribozyme self-cleavage site).

FIGS. 4A and 4B illustrate yet another method for excising one or more guide RNAs from a primary transcript. I corresponds to a Zea mays U6 promoter (SEQ ID NO:16), II to a DNA encoding a RNase Z recognition site (SEQ ID NO:17-18), III to a DNA encoding a gRNA Cas Recognition domain for SpCas12f1 (SEQ ID NO:19), IV to a DNA encoding a SpCas12f1 gRNA Variable Targeting domain (SEQ ID NO:20-24), and VI to a Zea mays U6 terminator (SEQ ID NO: 26). FIG. 4B illustrates a DNA expression cassette comprising a DNA sequence encoding a guide RNA flanked by 5′ and 3′ DNA sequences encoding RNase Z recognition sites. FIG. 4A illustrates excision of a guide RNA from a primary transcript via RNase Z (scissors represent RNase Z cleavage sites).

FIGS. 5A-5C illustrate another method for excising one or more guide RNAs from a primary transcript. I corresponds to a Zea mays U6 promoter (SEQ ID NO:16), II to a DNA encoding a MS2 recognition site (SEQ ID NO:37), III to a DNA encoding an optional linker RNA (SEQ ID NO:39), IV to a DNA encoding a gRNA Cas Recognition domain for SpCas12f1 (SEQ ID NO: 19), V to a DNA encoding a SpCas12f1 gRNA Variable Targeting domain (SEQ ID NO: 20-24), VI to a Zea mays U6 terminator (SEQ ID NO:26), VII to a Zea mays Ubiquitin promoter (SEQ ID NO:8), VIII to a Zea mays Ubiquitin 5′ UTR (SEQ ID NO:9), IX to a Zea mays Ubiquitin intron 1 (SEQ ID NO:10), and X to a Zea mays Ubiquitin terminator (SEQ ID NO:15). FIG. 5B illustrates a polymerase III DNA expression cassette comprising a DNA sequence encoding a guide RNA flanked by 5′ and 3′ DNA sequences encoding MS2 coat protein recognition sequences in the form of stem loop-forming RNA. FIG. 5C illustrates a polymerase II DNA expression cassette comprising a DNA sequence encoding a guide RNA flanked by 5′ and 3′ DNA sequences encoding MS2 coat protein recognition sequences in the form of stem loop-forming RNA. FIG. 5A illustrates excision of a guide RNA from a primary transcript via the MS2 coat protein recognition sequences of the primary sequence complexing with two MS2-PIN fusion proteins (circles represent the MS2 coat protein and scissors represent the PIN ribonuclease linked domain).

FIGS. 6A-6C illustrate yet another method for excising one or more guide RNAs from a primary transcript. I corresponds to a Zea mays U6 promoter (SEQ ID NO:16), II to a DNA encoding a PP7 recognition site (SEQ ID NO:38), III to a DNA encoding an optional linker RNA (SEQ ID NO:39), IV to a DNA encoding a gRNA Cas Recognition domain for SpCas12f1 (SEQ ID NO: 19), V to a DNA encoding a SpCas12f1 gRNA Variable Targeting domain (SEQ ID NO: 20-24), VI to a DNA encoding a ribozyme (SEQ ID NO:25), VII to a Zea mays U6 terminator (SEQ ID NO:26), VIII to a Zea mays Ubiquitin promoter (SEQ ID NO:8), IX to a Zea mays Ubiquitin 5′ UTR (SEQ ID NO:9), X to a Zea mays Ubiquitin intron 1 (SEQ ID NO:10), and XI to a Zea mays Ubiquitin terminator (SEQ ID NO:15). FIG. 6B illustrates a polymerase III DNA expression cassette comprising a DNA sequence encoding a guide RNA flanked by 5′ and 3′ DNA sequences encoding PP7 coat protein recognition sequences in the form of stem loop-forming RNA. Alternatively, the DNA sequence encoding a guide RNA is flanked by a 5′ DNA sequence encoding a PP7 coat protein recognition sequence and a 3′ DNA sequence encoding a ribozyme. FIG. 6C illustrates a polymerase II DNA expression cassette comprising a DNA sequence encoding a guide RNA flanked by 5′ and 3′ DNA sequences encoding PP7 coat protein recognition sequences in the form of stem loop-forming RNA. Alternatively, the DNA sequence encoding a guide RNA is flanked by a 5′ DNA sequence encoding a PP7 coat protein recognition sequence and a 3′ DNA sequence encoding a ribozyme. FIG. 6A illustrates excision of a guide RNA from a primary transcript via the PP7 coat protein recognition sequences of the primary sequence complexing with monomeric PP7-PIN fusion proteins. Alternatively, excision of a guide RNA from a primary transcript occurs via the 5′ PP7 coat protein recognition sequence complexing with a monomeric PP7-PIN fusion protein and the 3′ self-cleaving ribozyme (circles represent the MS2 coat protein, scissors represent the PIN ribonuclease linked domain, and the triangle represents the ribozyme self-cleavage site).

FIGS. 7A-7E illustrate another method for excising one or more guide RNAs from a primary transcript. I corresponds to a Zea mays U6 promoter (SEQ ID NO:16), II to a DNA encoding a HH ribozyme (SEQ ID NO:42), III to a DNA encoding a gRNA Cas Recognition domain for SpCas12f1 (SEQ ID NO:19), IV to a DNA encoding a SpCas12f1 gRNA Variable Targeting domain (SEQ ID NO:20-24), V to a DNA encoding a HDV ribozyme (SEQ ID NO:25), VI to a Zea mays U6 terminator (SEQ ID NO:26), VII to a Zea mays Ubiquitin promoter (SEQ ID NO: 8), VIII to a Zea mays Ubiquitin 5′ UTR (SEQ ID NO:9), IX to a Zea mays Ubiquitin intron 1 (SEQ ID NO:10), X to a Zea mays Ubiquitin terminator (SEQ ID NO:15), and XI to a DNA encoding an optional flexible RNA (5′-CUUG-3′ or SEQ ID NO:43). FIG. 7B illustrates a polymerase III DNA expression cassette comprising a DNA sequence encoding a guide RNA flanked by a 5′ DNA sequence encoding a first ribozyme, and a 3′ DNA sequence encoding a second ribozyme. FIG. 7C illustrates a polymerase II DNA expression cassette comprising a DNA sequence encoding a guide RNA flanked by a 5′ DNA sequence encoding a first ribozyme and a 3′ DNA sequence encoding a second ribozyme. FIG. 7A illustrates excision of a guide RNA from a primary transcript via the first and second self-cleaving ribozymes (triangles represent the ribozyme self-cleavage site). FIG. 7E illustrates a polymerase III DNA expression cassette comprising a first DNA sequence encoding a first guide RNA and a second DNA sequence encoding a second guide RNA, wherein each DNA sequence encoding a guide RNA is flanked by a 5′ DNA sequence encoding a first ribozyme and a 3′ DNA sequence encoding a second ribozyme. FIG. 7D illustrates excision of a first and a second guide RNA from a primary transcript via the self-cleaving first and second ribozymes (triangles represent the ribozyme self-cleavage site).

FIGS. 8A and 8B illustrate yet another method for excising one or more guide RNAs from a primary transcript. I corresponds to a Zea mays U6 promoter (SEQ ID NO:16), II to a DNA encoding a gRNA Cas Recognition domain for SpCas12f1 (SEQ ID NO:19), III to a DNA encoding a SpCas12f1 gRNA Variable Targeting domain (SEQ ID NO:20-24), IV to a DNA encoding a HDV ribozyme (SEQ ID NO:25), V to a DNA encoding an optional flexible RNA (5′-CUUG-3′ or SEQ ID NO:43), VI to a Zea mays U6 terminator (SEQ ID NO:26). FIG. 8B illustrates a DNA expression cassette comprising a first DNA sequence encoding a first guide RNA and a second DNA sequence encoding a second guide RNA, wherein the 3′ end of the first and second guide RNAs are flanked by a DNA sequence encoding a ribozyme. FIG. 8A illustrates excision of a first and a second guide RNA from a primary transcript via the self-cleaving ribozymes (triangles represent the ribozyme self-cleavage site).

FIGS. 9A and 8B illustrate another method for excising one or more guide RNAs from a primary transcript. A corresponds to a Zea mays U6 promoter (SEQ ID NO:16), B to a DNA encoding a gRNA Cas Recognition domain for SpCas12f1 (SEQ ID NO: 19), C to a DNA encoding a SpCas12f1 gRNA Variable Targeting domain (SEQ ID NO:20-24), D to a DNA encoding an RNase III stem-loop recognition sequence (SEQ ID NO: 67-70), E to a DNA encoding an optional flexible RNA (5′-CUUG-3′ or SEQ ID NO:43), F to a Zea mays U6 terminator (SEQ ID NO:26). FIG. 9B illustrates a DNA expression cassette comprising a first DNA sequence encoding a first guide RNA and a second DNA sequence encoding a second guide RNA, wherein the 3′ end of the first and second guide RNAs are flanked by a DNA sequence encoding an RNase III stem-loop recognition sequence. FIG. 9A illustrates excision of a first and a second guide RNA from a primary transcript via RNase III (triangles represent cleavage sites).

FIGS. 10A and 10B illustrate the formation of a rabbit ear stem loop from rRNA.

FIG. 11 illustrates the Rnt1 homodimer.

FIGS. 12A-12D illustrate a maize-optimized RNase III heterodimer.

FIGS. 13A-13D illustrate a maize-optimized RNase III heterodimer having a linker polypeptide.

FIG. 14 is a graph illustrating targeted mutagenesis of five genomic sites. 1: negative control, guide RNA expression construct omitted; five gRNA expressed from a separate U6 promoter, gRNA-HDV design; 3: five gRNAs expressed from a single U6 promoter, rice RNase Z-gRNA-HDV design; 4: five gRNAs expressed from a single U6 promoter, maize RNase Z-gRNA-HDV design; 5: five gRNAs expressed from a single UBI promoter, rice RNase Z-gRNA-HDV design; 6: five gRNAs expressed from a single UBI promoter, maize RNase Z-gRNA-HDV design; 7: five gRNAs expressed from a single UBI promoter, rice RNase Z-gRNA-rice RNase Z design; 8: five gRNAs expressed from a single UBI promoter, maize RNase Z-gRNA-maize RNase Z design.

FIG. 15 is a graph illustrating the editing frequencies at five genomic target sites (FIG. 14) averaged for each treatment and normalized to the average editing frequency of a positive control. 1: negative control, guide RNA expression construct omitted; five gRNA expressed from a separate U6 promoter, gRNA-HDV design; 3: five gRNAs expressed from a single U6 promoter, rice RNase Z-gRNA-HDV design; 4: five gRNAs expressed from a single U6 promoter, maize RNase Z-gRNA-HDV design; 5: five gRNAs expressed from a single UBI promoter, rice RNase Z-gRNA-HDV design; 6: five gRNAs expressed from a single UBI promoter, maize RNase Z-gRNA-HDV design; 7: five gRNAs expressed from a single UBI promoter, rice RNase Z-gRNA-rice RNase Z design; 8: five gRNAs expressed from a single UBI promoter, maize RNase Z-gRNA-maize RNase Z design.

SEQ ID NO: 1 is the Cas9 PRT sequence from Streptococcus pyogenes (SpyCas9).

SEQ ID NO: 2 is the Cas12f1 PRT sequence from Syntrophomonas palmitica (SpaCas12f1).

SEQ ID NO: 3 is a first engineered variant (artificial) of the Cas12f1 PRT sequence from Syntrophomonas palmitica.

SEQ ID NO: 4 is a second engineered variant (artificial) of the Cas12f1 PRT sequence from Syntrophomonas palmitica.

SEQ ID NO: 5 is the Cas12f1 PRT sequence from Acidibacillus sulfuroxidans (AsuCas12f1).

SEQ ID NO: 6 is an engineered variant (artificial sequence) of the Cas12f1 PRT sequence from Acidibacillus sulfuroxidans.

SEQ ID NO: 7 is a VirD2 nuclear localization signal PRT sequence from Agrobacterium tumefaciens.

SEQ ID NO: 8 is a Zea mays UBI promoter DNA sequence.

SEQ ID NO: 9 is a Zea mays UBI 5′ UTR DNA sequence.

SEQ ID NO: 10 is a Zea mays UBI Intron 1 DNA sequence.

SEQ ID NO: 11 is a first maize-optimized engineered variant (artificia) of the Cas12f1 DNA sequence from Syntrophomonas palmitica.

SEQ ID NO: 12 is a second maize-optimized engineered variant (artificial) of the Cas12f1 DNA sequence from Syntrophomonas palmitica.

SEQ ID NO: 13 is a ST-LS1 Intron 2 DNA sequence from Solanum tuberosum.

SEQ ID NO: 14 is a maize-optimized engineered variant (artificial) DNA sequence of the SV40 NLS.

SEQ ID NO: 15 is a Zea mays UBI terminator DNA sequence.

SEQ ID NO: 16 is a Zea mays U6 promoter DNA sequence including a 3′ G to promote transcription.

SEQ ID NO: 17 is a DNA sequence encoding a Oryza sativa RNase Z recognition site.

SEQ ID NO: 18 is a DNA sequence encoding a Zea mays RNase Z recognition site.

SEQ ID NO: 19 is a DNA sequence encoding a gRNA Cas recognition domain for SpCas12f1.

SEQ ID NO: 20 is a DNA sequence encoding a SpCas12f1 gRNA Variable Targeting domain for a WUS2 site.

SEQ ID NO: 21 is a DNA sequence encoding a SpCas12f1 gRNA Variable Targeting domain for a CR82 site.

SEQ ID NO: 22 is a DNA sequence encoding a SpCas12f1 gRNA Variable Targeting domain for a 27b_WX4 site.

SEQ ID NO: 23 is a DNA sequence encoding a SpCas12f1 gRNA Variable Targeting domain for a CR36 site.

SEQ ID NO: 24 is a DNA sequence encoding a SpCas12f1 gRNA Variable Targeting domain for a 227b_MS1 site.

SEQ ID NO: 25 is an engineered variant (artificial) DNA sequence encoding a ribozyme derived from the Hepatitis Delta Virus.

SEQ ID NO: 26 is a Zea mays U6 terminator DNA sequence.

SEQ ID NO: 27 is a PIN ribonuclease domain PRT sequence from Homo sapiens.

SEQ ID NO: 28 is an artificial PRT sequence for a MS2 viral coat protein (N56K).

SEQ ID NO: 29 is a PRT sequence encoding a PP7 viral coat protein from Pseudomonas phage PP7.

SEQ ID NO: 30 is an artificial PRT sequence for a nuclear localization signal (NLS) containing a first linker.

SEQ ID NO: 31 is an artificial PRT sequence for a nuclear localization signal (NLS) containing a first linker.

SEQ ID NO: 32 is an artificial PRT sequence for a nuclear localization signal (NLS) containing a second linker.

SEQ ID NO: 33 is an artificial PRT sequence for a nuclear localization signal (NLS) containing a third linker.

SEQ ID NO: 34 is an artificial PRT sequence for a nuclear localization signal (NLS) containing a fourth linker.

SEQ ID NO: 35 is an artificial PRT sequence for a nuclear localization signal (NLS) containing a fifth linker.

SEQ ID NO: 36 is an artificial PRT sequence for a nuclear localization signal (NLS) containing a sixth linker.

SEQ ID NO: 37 is a DNA sequence encoding a stem-loop forming RNA capable of complexing with an MS2 coat protein.

SEQ ID NO: 38 is a DNA sequence encoding a stem-loop forming RNA capable of complexing with the PP7 coat protein.

SEQ ID NO: 39 is an artificial DNA sequence encoding a linker RNA.

SEQ ID NO: 40 is a Zea mays PLTP promoter DNA sequence.

SEQ ID NO: 41 is a Zea mays PLTP 5′ UTR DNA sequence.

SEQ ID NO: 42 is an artificial DNA sequence encoding a Hammerhead ribozyme.

SEQ ID NO: 43 is an artificial DNA sequence encoding a nexus-like trans-activating gRNA sequence.

SEQ ID NO: 44 is a Rnt1 DNA sequence from Saccharomyces cerevisiae.

SEQ ID NO: 45 is a Rnt1 PRT sequence from Saccharomyces cerevisiae.

SEQ ID NO: 46 is an engineered variant (artificial sequence) of the Rnt1 PRT sequence from Saccharomyces cerevisiae.

SEQ ID NO: 47 is an RNA sequence of a C8 stem-loop structure.

SEQ ID NO: 48 is an RNA sequence of a C-13 stem-loop structure.

SEQ ID NO: 49 is an RNA sequence of U2 snRNA from Saccharomyces cerevisiae.

SEQ ID NO: 50 is an RNA sequence of U5 snRNA from Saccharomyces cerevisiae.

SEQ ID NO: 51 is an RNA sequence of RPL7 RNA intron 2 from Saccharomyces cerevisiae.

SEQ ID NO: 52 is an RNA sequence of 25s RNA from Saccharomyces cerevisiae.

SEQ ID NO: 53 is a back-translated DNA sequence of a C8 stem-loop structure.

SEQ ID NO: 54 is a back-translated DNA sequence of a C-13 stem-loop structure.

SEQ ID NO: 55 is a back-translated DNA sequence of U2 snRNA from Saccharomyces cerevisiae.

SEQ ID NO: 56 is a back-translated DNA sequence of U5 snRNA from Saccharomyces cerevisiae.

SEQ ID NO: 57 is a back-translated DNA sequence of RPL7 RNA intron 2 from Saccharomyces cerevisiae.

SEQ ID NO: 58 is a back-translated DNA sequence of 25s RNA from Saccharomyces cerevisiae.

SEQ ID NO: 59 is a DNA sequence encoding Lambda N protein from Lambda bacteriophage.

SEQ ID NO: 60 is a PRT sequence for Lambda N protein from Lambda bacteriophage.

SEQ ID NO: 61 is a DNA sequence for a truncated N protein B-box.

SEQ ID NO: 62 is a PRT sequence for a truncated N protein B-box.

SEQ ID NO: 63 is a DNA sequence encoding Salmonella phage P22.

SEQ ID NO: 64 is a PRT sequence for Salmonella phage P22.

SEQ ID NO: 65 is a DNA sequence for a truncated P22 B-box.

SEQ ID NO: 66 is a PRT sequence for a truncated P22 B-box.

SEQ ID NO: 67 is a DNA sequence encoding Rnt1 recognition sequence C08.

SEQ ID NO: 68 is a DNA sequence encoding Rnt1 recognition sequence C13.

SEQ ID NO: 69 is a DNA sequence encoding Rnt1 recognition sequence C08r.

SEQ ID NO: 70 is a DNA sequence encoding Rnt1 recognition sequence C13r.

SEQ ID NO: 71 is a DNA sequence encoding Rnt1.

SEQ ID NO: 72 is a PRT sequence for Rnt1 RNase III enzyme.

SEQ ID NO: 73 is a DNA sequence encoding Rnt1 containing the ST-LS1 Intron 2 SEQ ID NO: 74 is a DNA sequence encoding a Zea mays U6 promoter including a 3′ G.

### DETAILED DESCRIPTION

The disclosures herein are described more fully hereinafter with reference to the accompanying figures, in which some, but not all possible aspects are shown. Indeed, disclosures can be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods and compositions pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed methods and compositions belong. In this specification and in the claims which follow, reference is made to a number of terms which shall be defined herein.

Terms used in the claims and specification are defined as set forth below unless otherwise specified. It must be noted that, as used in the specification and the appended claims, the singular forms “a,” “an” and “the” include plural referents unless the context clearly dictates otherwise.

As used herein, “nucleic acid” generally means a polynucleotide and includes a single or a double-stranded polymer of deoxyribonucleotide or ribonucleotide bases. Nucleic acids can also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

A “deaminase” is an enzyme that catalyzes a deamination reaction. For example, deamination of adenine with an adenine deaminase results in the formation of hypoxanthine. Hypoxanthine selectively base pairs with cytosine instead of thymine. This results in a post-replicative transition mutation, such that the original A-T base pair transforms into a G-C base pair. In another example, cytosine deamination results in the formation of uracil, which would normally be repaired by cellular repair mechanisms back to cytosine but can be prevented by the introduction of a uracil glycosylase inhibitor, such that DNA repair or replication transforms the original G-C base pair into an A-T base pair.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have structural similarity such that they are capable of acting as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed can be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Generally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides can be purified from a cell in which they naturally occur. Conventional nucleic acid purification methods known to skilled artisans can be used to obtain isolated polynucleotides. The term also embraces recombinant polynucleotides and chemically synthesized polynucleotides.

The term “fragment” refers to a contiguous set of nucleotides or amino acids. In some aspects, a fragment is 2, 3, 4, 5, 6, 78, 9, 10, 11, 12, 13, 14, 15, 16, 17, 18, 19, 20, or greater than 20 contiguous nucleotides. In some aspects, a fragment is 2, 3, 4, 5, 6, 78, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In some aspects, an endogenous polynucleotide is normally found in the genome of a cell; that is, not heterologous. As used herein, “endogenous target (nucleotide or polynucleotide) sequence”, “native target (nucleotide or polynucleotide) sequence”, and “wild-type (nucleotide or polynucleotide) sequence” can be used interchangeably and refer to a target nucleotide sequence that is endogenous or native to the genome of a plant and is at the endogenous or native position of that target sequence in the genome of the plant.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In some aspects of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

An “optimized nucleotide sequence” is a nucleotide sequence that has been optimized for expression in a particular organism. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and can be an innate element of the promoter or a heterologous element inserted to enhance the level or tissue-specificity of a promoter. Promoters can be derived in their entirety from a native gene, or be composed of different elements derived from different promoters found in nature, and/or comprise synthetic DNA segments. It is understood by those skilled in the art that different promoters can direct the expression of a gene in different tissues or cell types, or at different stages of development, or in response to different environmental conditions. It is further recognized that since in most cases the exact boundaries of regulatory sequences have not been completely defined, DNA fragments of some variation can have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence can affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” or refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some aspects, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements can be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host. In some aspects, a “Donor DNA cassette” comprises a heterologous polynucleotide to be inserted at the double-strand break site created by a double-strand-break inducing agent (e.g., a Cas endonuclease and guide polynucleotide complex), that is operably linked to a noncoding expression regulatory element. In some aspects, the Donor DNA cassette further comprises polynucleotide sequences that are homologous to the target site, that flank the polynucleotide of interest operably linked to a noncoding expression regulatory element.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct can comprise regulatory sequences and coding sequences that are derived from different sources, or regulatory sequences and coding sequences derived from the same source, but arranged in a manner different than that found in nature. Such a construct can be used by itself or can be used in conjunction with a vector. If a vector is used, then the choice of vector is dependent upon the method that will be used to introduce the vector into the host cells as is well known to those skilled in the art.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein can be entirely synthetic. In some aspects, a discrete component of a poly-gRNA molecule is heterologous to at least one other component, i.e., do not occur together in nature.

The term “expression”, as used herein, refers to the production of a functional end-product (e.g., an mRNA, guide polynucleotide, or a protein) in either precursor or mature form.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides can be, but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can include of a CRISPR array, comprising short direct repeats (CRISPR repeats) separated by short variable DNA sequences (called spacers), which can be flanked by diverse Cas (CRISPR-associated) genes.

As used herein, an “effector”, an “effector protein”, or an “effector polypeptide” is a protein that encompasses an activity including recognizing, binding to, and/or cleaving or nicking a polynucleotide target. An effector, or effector protein, can also be an endonuclease. The “effector complex” of a CRISPR system includes Cas proteins involved in crRNA and target recognition and binding. Some of the component Cas proteins can additionally comprise domains involved in target polynucleotide cleavage.

As used herein, “Cas protein” and “Cas polypeptide” refer to a polypeptide encoded by a Cas (CRISPR-associated) gene. Cas proteins include, but are not limited to, Cas9, Cpf1 (Cas12), C2c1, C2c2, C2c3, Cas3, Cas3-HD, Cas 5, Cas7, Cas8, Cas10, Cas-alpha, and combinations or complexes thereof. A Cas protein can be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The endonucleases of the disclosure can include those having one or more RuvC nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

A “Cas endonuclease” can comprise domains that enable it to function as a double-strand-break-inducing agent. A “Cas endonuclease” can also comprise one or more modifications or mutations that abolish or reduce its ability to cleave a double-strand polynucleotide (dCas). In some aspects, the Cas endonuclease molecule can retain the ability to nick a single-strand polynucleotide (for example, a D10A mutation in a Cas9 endonuclease molecule) (nCas9).

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double-strand break in) the target site is retained. The portion or subsequence of the Cas endonuclease can comprise a complete or partial (functional) peptide of any one of its domains such as for example, but not limiting to a complete of functional part of a Cas3 HD domain, a complete of functional part of a Cas3 Helicase domain, complete of functional part of a Cascade protein (such as but not limiting to a Cas5, Cas5d, Cas7 and Cas8b1).

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease can also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a cascade (comprises at least a second protein domain that can form a cascade with other proteins). In some aspects, the multifunctional Cas endonuclease comprises at least one additional protein domain relative (either internally, upstream (5′), downstream (3′), or both internally 5′ and 3′, or any combination thereof) to those domains typical of a Cas endonuclease.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “functional fragment”, “fragment that is functionally equivalent” and “functionally equivalent fragment” of a guide RNA, guide polynucleotide, crRNA, or tracrRNA are used interchangeably herein, and refer to a portion or subsequence of the guide RNA, guide polynucleotide, crRNA, or tracrRNA, respectively, of the present disclosure in which the ability to function as a guide RNA, guide polynucleotide, crRNA, or tracrRNA, respectively, is retained.

The terms “functional variant”, “variant that is functionally equivalent” and “functionally equivalent variant” of a guide RNA, guide polynucleotide, crRNA, or tracrRNA (respectively) are used interchangeably herein, and refer to a variant of the guide RNA, guide polynucleotide, crRNA, or tracrRNA, respectively, of the present disclosure in which the ability to function as a guide RNA, guide polynucleotide, crRNA, or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some aspects, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence, “target DNA”, “target locus”, “genomic target site”, “genomic target sequence”, “genomic target locus”, “target polynucleotide”, and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant cells comprise a plant cell wall, and as such are distinct, with different biochemical characteristics, from protoplasts that lack a cell wall.

A “plant element” or “plant part” is intended to reference either a whole plant or a plant component, which can comprise differentiated and/or undifferentiated tissues, for example but not limited to plant tissues, parts, and cell types. In some aspects, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue), plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides. The term “plant organ” refers to plant tissue or a group of tissues that constitute a morphologically and functionally distinct part of a plant. As used herein, a “plant element” is synonymous to a “portion” or “part” of a plant, and refers to any part of the plant, and can include distinct tissues and/or organs, and can be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element can be in plant or in a plant organ, tissue culture, or cell culture.

“Progeny” comprises any subsequent generation of a plant.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos can be separated into two different groups, one receiving a treatment (such as the introduction of a CRISPR-Cas effector endonuclease) and one control that does not receive such treatment. Any phenotypic differences between the two groups can thus be attributed solely to the treatment and not to any inherency of the plant's endogenous genetic makeup.

A “population” of plants refers to a plurality of individual plants that share temporal and spatial location, and can further share one or more characteristic(s), such as a common genotype.

“Introducing” or “providing” are intended to mean presenting a subject molecule to a target, such as a cell or organism, a polynucleotide or polypeptide or polynucleotide-protein complex, in such a manner that the subject gains access to the target, such as the interior of a cell of the organism or to the cell itself, or in the case of a target polynucleotide, presented to the polynucleotide in such a way that the subject has capability of physical or chemical contact with the polynucleotide.

A “polynucleotide of interest” includes any nucleotide sequence that

In some aspects, a “polynucleotide of interest” encodes a protein or polypeptide that is “of interest” for a particular purpose, e.g., a selectable marker. In some aspects a trait or polynucleotide “of interest” is one that improves a desirable phenotype of a plant, particularly a crop plant, i.e., a trait of agronomic interest. Polynucleotides of interest: include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest can additionally be utilized in either the sense or anti-sense orientation. Further, more than one polynucleotide of interest can be utilized together, or “stacked”, to provide additional benefit. In some aspects, a “polynucleotide of interest” can encode a gene expression regulatory element, for example a promoter, intron, terminator, 5′UTR, 3′UTR, or other noncoding sequence. In some aspects, a “polynucleotide of interest” can comprise a DNA sequence that encodes for an RNA molecule, for example a functional RNA, siRNA, miRNA, guide polynucleotide, or a guide RNA that is capable of interacting with a Cas endonuclease to bind to a target polynucleotide sequence.

A “complex trait locus” includes a genomic locus that has multiple transgenes genetically linked to each other.

The compositions and methods herein can provide for an improved “agronomic trait” or “trait of agronomic importance” or “trait of agronomic interest” to a plant, which can include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic can be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase can be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “microliters” means microliter(s), “mL” means milliliter(s), “L” means liter(s), “uM” means micromolar, “mM” means millimolar, “M” means molar, “mmol” means millimole(s), “umole” mean micromole(s), “g” means gram(s), “micrograms” or “ug” means microgram(s), “ng” means nanogram(s), “U” means unit(s), “bp” means base pair(s) and “kb” means kilobase(s).

In a first aspect, the disclosure provides methods of delivering a plurality of guide polynucleotides for multiplexed genome editing of multiple genomic targets in a plant cell.

In some aspects, a method of delivering a plurality of guide polynucleotides for multiplexed genome editing of multiple genomic targets in a plant cell comprises: providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop.

In some aspects, a method of delivering a plurality of guide polynucleotides for multiplexed genome editing of multiple genomic targets in a plant cell comprises: providing a polynucleotide molecule to the plant cell, the polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In some aspects, a method of delivering a plurality of guide polynucleotides for multiplexed genome editing of multiple genomic targets in a plant cell comprises: providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop.

In some aspects, a method of delivering a plurality of guide polynucleotides for multiplexed genome editing of multiple genomic targets in a plant cell comprises: providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by an RNase Z recognition sequence; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In some aspects, a method of delivering a plurality of guide polynucleotides for multiplexed genome editing of multiple genomic targets in a plant cell comprises: providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence; and expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

In some aspects, a method of delivering a plurality of guide polynucleotides for multiplexed genome editing of multiple genomic targets in a plant cell comprises providing to the plant cell a polynucleotide expression cassette encoding two or more guide RNA sequences separated by one or more tRNA cleavage sequence, wherein each guide RNA sequence comprises a 3′ spacer sequence that exhibits complementarity to a target sequence in the plant cell, wherein the plant cell's tRNA processing system cleaves a transcript generated from the transcribed polynucleotide expression cassette, thereby generating the plurality of guide RNA sequences.

In a second aspect, the disclosure provides compositions, and plant cells comprising the compositions, for delivering a plurality of guide polynucleotides for multiplexed genome editing of multiple genomic targets in a plant cell.

In some aspects, a composition for delivering a plurality of guide polynucleotides for multiplexed genome editing of multiple genomic targets in a plant cell comprises: (a) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and (b) a heterologous eukaryotic RNase III that cleaves an RNA molecule transcribed from the polynucleotide molecule, wherein the RNA molecule having the RNase III recognition sequence comprises at least one stem loop.

In some aspects, a composition for delivering a plurality of guide polynucleotides for multiplexed genome editing of multiple genomic targets in a plant cell comprises: a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotides is flanked by the RNase recognition sequence at a 5′ end.

In some aspects, a composition for delivering a plurality of guide polynucleotides for multiplexed genome editing of multiple genomic targets in a plant cell comprises: (a) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and (b) a double-stranded RNA-specific protein comprising a ribonuclease domain that cleaves an RNA molecule transcribed from the polynucleotide molecule, wherein the RNA molecule having the double-stranded RNA-specific protein recognition sequence comprises at least one stem loop.

In some aspects, a composition for delivering a plurality of guide polynucleotides for multiplexed genome editing of multiple genomic targets in a plant cell comprises: a polynucleotide molecule comprising at least two guide polynucleotide sequences targeting at least two distinct genomic target sites in the plant cell, wherein each of the at least two guide polynucleotide sequences is flanked by an RNase recognition sequence.

In some aspects, a composition for delivering a plurality of guide polynucleotides for multiplexed genome editing of multiple genomic targets in a plant cell comprises: a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence.

In some aspects, a composition for delivering a plurality of guide polynucleotides for multiplexed genome editing of multiple genomic targets in a plant cell comprises a nucleic acid expression cassette for generating a plurality of guide RNA molecules for genetic modification in a plant cell comprising a polynucleotide encoding two or more guide RNA sequences separated by one or more tRNA cleavage sequence, wherein each guide RNA sequence comprises a 3′ spacer sequence that exhibits complementarity to a target sequence in the plant cell.

In a third aspect, the disclosure provides methods for editing a plant genome.

In some aspects, a method for editing a plant genome comprises: providing a plant cell with (a) a Cas endonuclease; and (b) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects, a method for editing a plant genome comprises: providing a plant cell with (a) a Cas endonuclease, and (b) a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end; expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects, a method for editing a plant genome comprises: providing a plant cell with (a) a Cas endonuclease, and (b) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects, a method for editing a plant genome comprises: providing a plant cell with (a) a Cas endonuclease, and (b) a polynucleotide molecule comprising at least two guide polynucleotide sequences targeting at least two distinct genomic target sites in the plant cell, wherein each of the at least two guide polynucleotide sequences is flanked by an RNase Z recognition sequence; expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

In some aspects, a method for editing a plant genome comprises: providing a plant cell with (a) a Cas endonuclease, and (b) a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence; expressing the polynucleotide molecule in the plant cell to cleave an RNA molecule transcribed from the polynucleotide molecule, thereby delivering the at least two guide polynucleotide sequences as a first guide polynucleotide and a second guide polynucleotide; introducing a first site-specific modification in a first target nucleotide sequence in the plant cell when the Cas endonuclease complexes with the first guide polynucleotide; and introducing a second site-specific modification in a second target nucleotide sequence in the plant cell when the Cas endonuclease complexes with the second guide polynucleotide.

### CRISPR-Cas

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide polynucleotide) that is in complex with the Cas endonuclease. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein can lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases of the methods, compositions, and cells described herein include, but are not limited to, for example: Cas3 (a feature of Class 1 type I systems), Cas9 (a feature of Class 2 type II systems), Cpf1 (a feature of Class 2 type V systems), and Cas-alpha.

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

A Cas endonuclease, effector protein, or functional fragment thereof, for use in the disclosed methods, can be isolated from a native source, or from a recombinant source where the genetically modified host cell is modified to express the nucleic acid sequence encoding the protein. Alternatively, the Cas endonuclease protein can be produced using cell free protein expression systems, or be synthetically produced. Cas endonucleases can be isolated and introduced into a heterologous cell, or can be modified from its native form to exhibit a different type or magnitude of activity than what it would exhibit in its native source. Such modifications include but are not limited to: fragments, variants, substitutions, deletions, and insertions.

Fragments and variants of Cas endonucleases can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas endonuclease can be fused to a heterologous sequence to induce or modify activity.

A Cas endonuclease can be part of a fusion protein comprising one or more heterologous protein domains (e.g., 1, 2, 3, or more domains in addition to the Cas protein. Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas can also be fused to a FokI nuclease to generate double-strand breaks (Guilinger et al. Nature Biotechnology, volume 32, number 6, June 2014). In some aspects, the Cas endonuclease is a fusion protein further comprising a nuclease domain, a transcriptional activator domain, a transcriptional repressor domain, an epigenetic modification domain, a cleavage domain, a nuclear localization signal, a cell-penetrating domain, a translocation domain, a marker, or a transgene that is heterologous to the target polynucleotide sequence or to the cell from which said target polynucleotide sequence is obtained or derived. In some aspects, the nuclease fusion protein comprises Clo51 or Fok1.

The Cas endonucleases described herein can be expressed and purified by methods known in the art, for example as described in WO/2016/186953 published 24 Nov. 2016.

A Cas endonuclease can comprise a heterologous nuclear localization sequence (NLS). A heterologous NLS amino acid sequence herein can be of sufficient strength to drive accumulation of a Cas protein in a detectable amount in the nucleus of a yeast cell herein, for example.

In some aspects of the methods and compositions disclosed herein, a Cas endonuclease gene can be plant-optimized, wherein the plant-optimized Cas endonuclease is capable of binding to and creating a double strand break in a genomic target sequence of a plant genome. As used herein, a “plant-optimized Cas endonuclease” (e.g., “plant-optimized Cas9 endonuclease”, “plant-optimized Cas-alpha endonuclease”, and “plant-optimized Cas12f endonuclease”) refers to a Cas endonuclease encoded by a nucleotide sequence that has been optimized for expression in a plant cell or a plant. A “plant-optimized nucleotide sequence encoding a Cas endonuclease” and a “plant-optimized construct encoding a Cas endonuclease” are used interchangeably herein and refer to a nucleotide sequence encoding a Cas endonuclease polypeptide, or a variant or functional fragment thereof, that has been optimized for expression in a plant cell or plant. A plant comprising a plant-optimized Cas endonuclease includes a plant comprising the nucleotide sequence encoding for the Cas polypeptide sequence and/or a plant comprising the Cas endonuclease polypeptide. In some aspects, a plant-optimized Cas endonuclease nucleotide sequence results in increased Cas polypeptide expression when compared to the wild-type sequence of which it was optimized from. In some aspects, a plant-optimized nucleotide sequence encoding a Cas endonuclease can be a maize-optimized, canola-optimized, sunflower-optimized, rice-optimized, wheat-optimized, or soybean-optimized Cas endonuclease.

Additional sequence modifications are known to enhance gene expression in a plant host. These include, for example, elimination of one or more sequences encoding spurious polyadenylation signals, one or more exon-intron splice site signals, one or more transposon-like repeats, and other such well-characterized sequences that can be deleterious to gene expression. The G-C content of the sequence can be adjusted to levels average for a given plant host, as calculated by reference to known genes expressed in the host plant cell. When possible, the sequence is modified to avoid one or more predicted hairpin secondary mRNA structures. Thus, “a plant-optimized” of the present disclosure can include one or more of such sequence modifications.

### Cas9 Endonuclease

In some aspects of the methods and compositions disclosed herein, the genome editing system comprises a Cas9 endonuclease and one or more guide polynucleotides that introduce one or more site-specific modifications in the nucleotide sequence of one or more regulatory elements of a plant cell. In some aspects of the methods and compositions disclosed herein, the genome editing system comprises a Cas9 endonuclease, one or more guide polynucleotides, and a donor DNA. Some exemplary Cas9 endonucleases are described, for example, in WO2019165168.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. The canonical Cas9 recognizes a 3′ GC-rich PAM sequence on a target dsDNA, typically comprising an NGG motif. The Cas endonucleases described herein may recognize additional PAM sequences and be used to modify target sites with different recognition sequence specificity.

A Cas9 protein comprises a RuvC nuclease with an HNH (H—N—H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide polynucleotide (Makarova et al. 2015, Nature Reviews Microbiology Vol. 13:1-15).

A Cas9 endonuclease, effector protein, or functional fragment thereof, for use in the disclosed methods and compositions, can be isolated from a native source, or from a recombinant source where the genetically modified host cell is modified to express the nucleic acid sequence encoding the protein. Alternatively, the Cas endonuclease protein can be produced using cell free protein expression systems or be synthetically produced. Cas endonucleases can be isolated and introduced into a heterologous cell or can be modified from its native form to exhibit a different type or magnitude of activity than what it would exhibit in its native source. Such modifications include, but are not limited to, fragments, variants, substitutions, deletions, and insertions.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that interacts with a Cas endonuclease.

### Cas-Alpha Endonuclease

In some aspects of the methods and compositions disclosed herein, the genome editing system comprises a Cas-alpha (e.g., Cas12f) endonuclease and one or more guide polynucleotides that introduce one or more site-specific modifications in the nucleotide sequence of one or more regulatory elements of a plant cell. In some aspects of the methods and compositions disclosed herein, the genome editing system comprises a Cas-alpha endonuclease, one or more guide polynucleotides, and a donor DNA. Some exemplary Cas-alpha endonucleases are described, for example, in U.S. Pat. No. 10,934,536 and WO2022082179.

A Cas-alpha endonuclease is a functional RNA-guided, PAM-dependent dsDNA cleavage protein of fewer than 800 amino acids, comprising: a C-terminal RuvC catalytic domain split into three subdomains and further comprising bridge-helix and one or more Zinc finger motif(s); and an N-terminal Rec subunit with a helical bundle, WED wedge-like (or “Oligonucleotide Binding Domain”, OBD) domain, and, optionally, a Zinc finger motif.

Cas-alpha endonucleases comprise one or more Zinc Finger (ZFN) coordination motif(s) that may form a Zinc binding domain. Zinc Finger-like motifs can aid in target and non-target strand separation and loading of the guide polynucleotide into the DNA target. Cas-alpha endonucleases comprising one or more Zinc Finger motifs can provide additional stability to a ribonucleoprotein complex on a target polynucleotide. Cas-alpha endonucleases comprise C4 or C3H zinc binding domains.

A Cas-alpha endonuclease can function as a double-strand-break-inducing agent, a single-strand-break inducing agent, or as a nickase. In some aspects, a catalytically inactive Cas-alpha endonuclease can be used to target or recruit to a target DNA sequence but not induce cleavage. In some aspects, a catalytically inactive Cas-alpha protein can be combined with a base editing molecule, such as a cytidine deaminase or an adenine deaminase.

A Cas-alpha endonuclease, effector protein, or functional fragment thereof, can be used in the disclosed methods and compositions for targeted genome editing (via simplex and multiplex double-strand breaks and nicks). In some aspects of the methods and compositions disclosed herein, a genome editing system comprises Cas12f.

### Protospacer Adjacent Motif

A “protospacer adjacent motif” (PAM) herein refers to a short nucleotide sequence adjacent to a target sequence (protospacer) that can be recognized (targeted) by a guide polynucleotide/Cas endonuclease system. In some aspects, the Cas endonuclease may not successfully recognize a target DNA sequence if the target DNA sequence is not adjacent to, or near, a PAM sequence. In some aspects, the PAM precedes the target sequence (e.g., Cas12a). In some aspects, the PAM follows the target sequence (e.g., S. pyogenes Cas9). The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T. Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position.

### Guide Polynucleotide/Cas Endonuclease Complexes

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide can be engineered or synthetic.

“Guide polynucleotide” includes a chimeric non-naturally occurring guide polynucleotide comprising regions that are not found together in nature (i.e., they are heterologous with respect to each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences.

In some aspects, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides.

The tracrRNA (trans-activating CRISPR RNA) comprises, in the 5′-to-3′ direction, (i) an “anti-repeat” sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015). In some aspects, the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides.

In some aspects, the RNA that guides the RNA/Cas endonuclease complex is a duplexed RNA comprising a duplex crRNA-tracrRNA.

In some aspects, the guide polynucleotide is a guide polynucleotide capable of forming a PGEN as described herein, wherein said guide polynucleotide comprises a first nucleotide sequence domain that is complementary to a nucleotide sequence in a target DNA, and a second nucleotide sequence domain that interacts with said Cas endonuclease polypeptide.

In some aspects, the guide polynucleotide is a guide polynucleotide described herein, wherein the first nucleotide sequence and the second nucleotide sequence domain is selected from the group consisting of a DNA sequence, a RNA sequence, and a combination thereof.

In some aspects, the guide polynucleotide is a guide polynucleotide described herein, wherein the first nucleotide sequence and the second nucleotide sequence domain is selected from the group consisting of RNA backbone modifications that enhance stability, DNA backbone modifications that enhance stability, and a combination thereof (see Kanasty et al., 2013, Common RNA-backbone modifications, Nature Materials 12:976-977; US20150082478 published 19 Mar. 2015 and US20150059010 published 26 Feb. 2015)

The guide RNA includes a dual molecule comprising a chimeric non-naturally occurring crRNA linked to at least one tracrRNA. A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide can be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site. (US20150082478 published 19 Mar. 2015 and US20150059010 published 26 Feb. 2015).

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The nucleotide sequence linking the crNucleotide and the tracrNucleotide of a single guide polynucleotide can comprise a RNA sequence, a DNA sequence, or a RNA-DNA combination sequence. In some aspects, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In some aspects, the nucleotide sequence linking the crNucleotide and the tracrNucleotide of a single guide polynucleotide can comprise a tetraloop sequence, such as, but not limiting to a GAAA tetraloop sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limited to Xie et al. 2015, PNAS 112:3570-3575).

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41:4336-4343; Ma et al., Mol. Ther. Nucleic Acids 3: e161). This strategy has been successfully applied in cells of several different species including maize and soybean (U.S. Pat. No. 20150082478, published on Mar. 19, 2015). Methods for expressing RNA components that do not have a 5′ cap have been described (WO 2016/025131, published on Feb. 18, 2016).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas9 system that can form a complex with a type II Cas9 endonuclease, wherein said guide RNA/Cas9 endonuclease complex can direct the Cas9 endonuclease to a DNA target site, enabling the Cas9 endonuclease to recognize, bind to, and optionally nick or cleave (introduce a single or double strand break) the DNA target site.

Single guide RNAs targeting a target site in the genome of an organism can be designed by changing the Variable Targeting Domain (VT) of any of the guide polynucleotides described herein, with any random nucleotide that can hybridize to any desired target sequence.

In some aspects, a subject nucleic acid (e.g., a guide polynucleotide, a nucleic acid comprising a nucleotide sequence encoding a guide polynucleotide; a nucleic acid encoding Cas9 endonuclease of the present disclosure; a crRNA or a nucleotide encoding a crRNA, a tracrRNA or a nucleotide encoding a tracrRNA, a nucleotide encoding a VT domain, a nucleotide encoding a CER domain, etc.) comprises a modification or sequence that provides for an additional desirable feature (e.g., modified or regulated stability; subcellular targeting; tracking, e.g., a fluorescent label; a binding site for a protein or protein complex; etc.). Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

Functional variants of a guide polynucleotide of the present disclosure can comprise a modified guide polynucleotide wherein the modification comprises adding, removing, or otherwise altering loops and/or hairpins in the single guide RNA.

Functional variants of a guide polynucleotide of the present disclosure can comprise a modified guide polynucleotide wherein the modification comprises one or more modified nucleotides in the nucleotide sequence, wherein the one or more modified nucleotides comprises at least one non-naturally-occurring nucleotide, nucleotide mimetic (as described in US application US2014/0068797, published Mar. 6, 2014), or analog thereof, or wherein the one or more modified nucleotides are selected from the group consisting of 2′-O-methylanalogs, 2′-fluoro analogs 2-aminopurine, 5-bromo-uridine, pseudouridine, and 7-methylguanosine.

In some aspects, the functional variant of the guide RNA can form a guide RNA/Cas endonuclease complex that can recognize, bind to, and optionally nick or cleave a target sequence.

A guide polynucleotide/Cas endonuclease complex described herein is capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild-type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas nickase can comprise (i) a mutant, dysfunctional RuvC domain and (ii) a functional HNH domain (e.g., wild type HNH domain). As another example, a Cas nickase can comprise (i) a functional RuvC domain (e.g., wild type RuvC domain) and (ii) a mutant, dysfunctional HNH domain. Non-limiting examples of Cas nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these aspects can be at least 5, between 5 and 10, at least 10, between 10 and 15, at least 15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any number between 5 and 100) bases apart from each other, for example. One or two Cas nickase proteins herein can be used in a Cas nickase pair. For example, a Cas nickase with a mutant RuvC domain, but functioning HNH domain (i.e., Cas HNH+/RuvC−), can be used (e.g., Streptococcus pyogenes Cas HNH+/RuvC−). Each Cas nickase (e.g., Cas HNH+/RuvC−) can be directed to specific DNA sites nearby each other (up to 100 base pairs apart) by using suitable RNA components herein with guide polynucleotide sequences targeting each nickase to each specific DNA site.

A guide polynucleotide/Cas endonuclease complex in certain aspects can bind to a DNA target site sequence, but does not cleave any strand at the target site sequence. Such a complex can comprise a Cas protein in which all of its nuclease domains are mutant, dysfunctional. For example, a Cas protein that can bind to a DNA target site sequence, but does not cleave any strand at the target site sequence, can comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

In some aspects, the guide polynucleotide/Cas endonuclease complex is a guide polynucleotide/Cas endonuclease complex (PGEN) comprising at least one guide polynucleotide and at least one Cas endonuclease polypeptide. In some aspects, the Cas endonuclease polypeptide comprises at least one protein subunit of another Cas protein, or a functional fragment thereof, wherein said guide polynucleotide is a chimeric non-naturally occurring guide polynucleotide, wherein said guide polynucleotide/Cas endonuclease complex is capable of recognizing, binding to, and optionally nicking, unwinding, or cleaving all or part of a target sequence.

In some aspects, the PGEN is a ribonucleoprotein complex (RNP), wherein the Cas endonuclease is provided as a protein and the guide polynucleotide is provided as a ribonucleotide.

In some aspects, the guide polynucleotide/Cas effector complex is a guide polynucleotide/Cas endonuclease complex (PGEN) comprising at least one guide polynucleotide and a Cas endonuclease, wherein said guide polynucleotide/Cas endonuclease complex is capable of recognizing, binding to, and optionally nicking, unwinding, or cleaving all or part of a target sequence.

The PGEN can be a guide polynucleotide/Cas endonuclease complex, wherein said Cas endonuclease further comprises one copy or multiple copies of at least one protein subunit, or a functional fragment thereof, of an additional Cas protein.

In some aspects, the guide polynucleotide/Cas endonuclease complex (PGEN) described herein is a PGEN, wherein said Cas endonuclease is covalently or non-covalently linked to at least one Cas protein subunit, or functional fragment thereof. The PGEN can be a guide polynucleotide/Cas endonuclease complex, wherein said Cas endonuclease polypeptide is covalently or non-covalently linked, or assembled to one copy or multiple copies of at least one protein subunit, or a functional fragment thereof, of a Cas protein selected from the group consisting of a Cas1 protein subunit, a Cas2 protein subunit, a Cas4 protein subunit, and any combination thereof, in some aspects effectively forming a cleavage ready Cascade. The PGEN can be a guide polynucleotide/Cas endonuclease complex, wherein said Cas endonuclease is covalently or non-covalently linked or assembled to at least two different protein subunits of a Cas protein selected from the group consisting of a Cas1, a Cas2, and Cas4. The PGEN can be a guide polynucleotide/Cas endonuclease complex, wherein said Cas endonuclease is covalently or non-covalently linked to at least three different protein subunits, or functional fragments thereof, of a Cas protein selected from the group consisting of a Cas1, a Cas2, and Cas4, and any combination thereof.

Any component of the guide polynucleotide/Cas endonuclease complex, the guide polynucleotide/Cas endonuclease complex itself, as well as the polynucleotide modification template(s) and/or donor DNA(s), can be introduced into a heterologous cell or organism by any method known in the art.

Some uses for guide polynucleotide/Cas endonuclease systems include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Methods and compositions are provided herein for the chemical modification or alteration of one or more nucleobases of a target polynucleotide, to change the base(s) from one type to another, for example from a Cytosine to a Thymine or an Adenine to a Guanine, using an RNA-guided Cas endonuclease that has been modified to lack double- or single-strand cleaving activity.

### NHEJ and HDR

In some aspects of the methods and compositions described herein, the genome editing system comprises a Cas endonuclease, one or more guide polynucleotides, and optionally donor DNA, and editing a target regulatory element nucleotide sequence comprises nonhomologous end-joining (NHEJ) or homologous recombination (HR) following a Cas endonuclease-mediated double-strand break. Once a double-strand break is induced in the DNA, the cell's DNA repair mechanism is activated to repair the break. The most common repair mechanism to bring the broken ends together is the nonhomologous end-joining pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9). Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In some aspects of the methods and compositions described herein, the genome editing system comprises a Cas endonuclease, one or more guide polynucleotides, and a donor DNA. As used herein, “donor DNA” is a DNA construct that comprises a polynucleotide of interest to be inserted into the target site of a Cas endonuclease. Once a double-strand break is introduced in the target site by the endonuclease, the first and second regions of homology of the donor DNA can undergo homologous recombination with their corresponding genomic regions of homology resulting in exchange of DNA between the donor and the target genome. As such, the provided methods result in the integration of the polynucleotide of interest of the donor DNA into the double-strand break in the target site in the plant genome, thereby altering the original target site and producing an altered genomic target site.

### Base Editing

In some aspects of the methods and compositions described herein, the genome editing system comprises a base editing agent and a plurality of guide polynucleotides and editing a target regulatory element nucleotide sequence comprises introducing a plurality of nucleobase edits in the target regulatory element nucleotide sequence resulting in a variant nucleotide sequence.

One or more nucleobases of a target polynucleotide can be chemically altered, in some cases to change the base from one type to another, for example from a Cytosine to a Thymine, or an Adenine to a Guanine. In some aspects, a plurality of bases, for example 2 or more, 5 or more, 10 or more, 20 or more, 30 or more, 40 or more, 50 or more, 60 or more, 70 or more, 80 or more 90 or more, 100 or more, or even greater than 100, 200 or more, up to thousands of bases can be modified or altered, to produce a plant with a plurality of modified bases.

Any base editing complex, such as a base editing agent associated with an RNA-guided protein, can be used to target and bind to a desired locus in the genome of an organism and chemically modify one or more components of a target polynucleotide.

Site-specific base conversions can be achieved to engineer one or more nucleotide changes to create one or more edits into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016): 420-4. A catalytically “dead” or inactive Cas9 (dCas9), for example a catalytically inactive “dead” version of a Cas endonuclease disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA. Any molecule that effects a change in a nucleobase is a “base editing agent”.

For many traits of interest, the creation of single double-strand breaks and the subsequent repair via HDR or NHEJ is not ideal for quantitative traits. An observed phenotype includes both genotype effects and environmental effects. The genotype effects further comprise additive effects, dominance effects, and epistatic effects. The probability of no effect per any single edit can be greater than zero, and any single phenotypic effect can be small, depending on the method used and site selected. Double-stranded break repair can additionally be “noisy” and have low repeatability.

One approach to ameliorate the probability of no effect per edit or small phenotypic effect outcome is to multiplex genome modification, such that a plurality of target sites are modified. Methods to modify a genomic sequence that do not introduce double-strand breaks would allow for single base substitutions. Combining these approaches, multiplexed base editing is beneficial for creating large numbers of genotype edits that can produce observable phenotype modifications. In some cases, dozens or hundreds or thousands of sites can be edited within one or a few generations of an organism.

A multiplexed approach to base editing in an organism, has the potential to create a plurality of significant phenotypic variations in one or a few generations, with a positive directional bias to the effects. In some aspects, the organism is a plant. A plant or a population of plants with a plurality of edits can be cross-bred to produce progeny plants, some of which will comprise multiple pluralities of edits from the parental lines. In this way, accelerated breeding of desired traits can be accomplished in parallel in one or a few generations, replacing time-consuming traditional sequential crossing and breeding across multiple generations.

As used herein, “a deaminase” is an enzyme that catalyzes a deamination reaction. For example, deamination of adenine with an adenine deaminase results in the formation of hypoxanthine. Hypoxanthine selectively base pairs with cytosine instead of thymine. This results in a post-replicative transition mutation, such that the original A-T base pair transforms into a G-C base pair. In another example, cytosine deamination results in the formation of uracil, which would normally be repaired by cellular repair mechanisms back to cytosine but can be prevented introduction of a uracil glycosylase inhibitor, such that DNA repair or replication transforms the original G-C base pair into an A-T base pair.

A base editing deaminase, such as a cytidine deaminase or an adenine deaminase, can be fused to an RNA-guided endonuclease that can be deactivated (“dCas”, such as a deactivated Cas9) or partially active (“nCas”, such as a Cas9 nickase) so that it does not cleave a target site to which it is guided. The dCas forms a functional complex with a guide polynucleotide that shares homology with a polynucleotide sequence at the target site, and is further complexed with the deaminase molecule. The guided Cas endonuclease recognizes and binds to a double-stranded target sequence, opening the double-strand to expose individual bases. In the case of a cytidine deaminase, the deaminase deaminates the cytosine base and creates a uracil. Uracil glycosylase inhibitor (UGI) is provided to prevent the conversion of U back to C. DNA replication or repair mechanisms then convert the Uracil to a thymine (U to T), and subsequent repair of the opposing base (formerly G in the original G-C pair) to an Adenine, creating a T-A pair. For example, see Komor et al. Nature Volume 533, Pages 420-424, 19 May 2016.

### Prime Editing

In some aspects of the methods and compositions described herein, the genome editing system comprises a prime editing agent and a guide polynucleotide and editing a target regulatory element nucleotide sequence comprises introducing one or more insertions, deletions, or nucleobase swaps in a target regulatory element nucleotide sequence without generating a double-stranded DNA break.

In some aspects, the prime editing agent is a Cas polypeptide fused to a reverse transcriptase, wherein the Cas polypeptide is modified to nick DNA rather than generating double-strand break. This Cas-polypeptide-reverse transcriptase fusion can also be referred to as a “prime editor” or “PE”. In some aspects, the guide polynucleotide comprises a prime editing guide polynucleotide (pegRNA), and is larger than standard sgRNAs commonly used for CRISPR gene editing (e.g., >100 nucleobases). The pegRNA comprises a primer binding sequence (PBS) and a template containing the desired or target RNA sequence at its 3′ end.

During prime editing, the PE: pegRNA complex binds to a target DNA sequence and the modified Cas polypeptide nicks one target DNA strand resulting in a flap. The PBS on the pegRNA binds to the DNA flap and the target RNA sequence is reverse transcribed using the reverse transcriptase. The edited strand is incorporated into the target DNA at the end of the nicked flap, and the target DNA sequence is repaired with the new reverse transcribed DNA.

### Recombinant Constructs for Transformation of Cells

The disclosed guide polynucleotides, Cas endonucleases, deaminases, and guide various molecular systems disclosed herein, and any one combination thereof, optionally further comprising one or more polynucleotide(s) or polypeptide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

### Expression and Utilization of CRISPR-Cas Systems

The disclosure further provides expression constructs for expressing in a prokaryotic or eukaryotic cell/organism a guide polynucleotide/Cas system that is capable of recognizing, binding to, and optionally nicking, unwinding, or cleaving all or part of a target sequence.

In some aspects, the expression constructs of the disclosure comprise a promoter operably linked to a nucleotide sequence encoding a Cas gene (or optimized sequence, including a Cas endonuclease gene described herein) and a promoter operably linked to a guide polynucleotide of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

### Expression Elements

Any polynucleotide encoding a Cas endonuclease, guide polynucleotide, or other CRISPR system component disclosed herein can be functionally linked to a heterologous expression element, to facilitate transcription or regulation in a host cell. Such expression elements include but are not limited to: promoter, leader, intron, and terminator. Expression elements can be “minimal”-meaning a shorter sequence derived from a native source, that still functions as an expression regulator or modifier. Alternatively, an expression element can be “optimized”-meaning that its polynucleotide sequence has been altered from its native state in order to function with a more desirable characteristic in a particular host cell. Alternatively, an expression element can be “synthetic”-meaning that it is designed in silico and synthesized for use in a host cell. Synthetic expression elements can be entirely synthetic, or partially synthetic (comprising a fragment of a naturally-occurring polynucleotide sequence).

A method of expressing RNA components such as gRNA in eukaryotic cells for performing Cas-mediated DNA targeting has been to use RNA polymerase III (Pol III) promoters that allows for transcription of RNA with precisely defined, unmodified, 5′- and 3′-ends (DiCarlo et al., Nucleic Acids Res. 41:4336-4343; Ma et al., Mol. Ther. Nucleic Acids 3: e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

### Polynucleotides of Interest

Polynucleotides of interest can be endogenous to the organism being edited, or can be provided as heterologous molecules to the organism.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to molecules such as pesticides or herbicides), genes encoding proteins conferring resistance to biotic stress (such as attacks by fungi, viruses, bacteria, insects, or nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

Polynucleotide sequences of interest can encode proteins involved in providing disease or pest resistance. By “disease resistance” or “pest resistance” is intended that the plants avoid the harmful symptoms that are the outcome of the plant-pathogen interactions.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits can be introduced into plants by genes coding for resistance to herbicides that act to inhibit the action of acetolactate synthase (ALS, also referred to as acetohydroxyacid synthase, AHAS), in particular the sulfonylurea (UK: sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest can also comprise antisense sequences complementary to at least a portion of the messenger RNA (mRNA) for a targeted gene sequence of interest. Antisense nucleotides are constructed to hybridize with the corresponding mRNA. Modifications of the antisense sequences can be made as long as the sequences hybridize to and interfere with expression of the corresponding mRNA. In this manner, antisense constructions having 70%, 80%, or 85% sequence identity to the corresponding antisense sequences can be used. Furthermore, portions of the antisense nucleotides can be used to disrupt the expression of the target gene. Generally, sequences of at least 50 nucleotides, 100 nucleotides, 200 nucleotides, or greater can be used.

In addition, the polynucleotide of interest can also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Introduction of CRISPR-Cas System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid can be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (or crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3: e161; DiCarlo et al., 2013, Nucleic Acids Res. 41:4336-4343; WO2015026887, published 26 Feb. 2015). Any promoter capable of transcribing the guide polynucleotide in a cell can be used and includes a heat shock/heat inducible promoter operably linked to a nucleotide sequence encoding the guide polynucleotide.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

Introducing a guide polynucleotide/Cas endonuclease complex described herein, into a cell includes introducing the individual components of said complex either separately or combined into the cell, and either directly (direct delivery as RNA for the guide and protein for the Cas endonuclease and Cas protein subunits, or functional fragments thereof) or via recombination constructs expressing the components (guide polynucleotide, Cas endonuclease, Cas protein subunits, or functional fragments thereof). Introducing a guide polynucleotide/Cas endonuclease complex (RGEN) into a cell includes introducing the guide polynucleotide/Cas endonuclease complex as a ribonucleotide-protein into the cell. The ribonucleotide-protein can be assembled prior to being introduced into the cell as described herein. The components comprising the guide polynucleotide/Cas endonuclease ribonucleotide protein (at least one Cas endonuclease, at least one guide polynucleotide, at least one Cas protein subunits) can be assembled in vitro or assembled by any means known in the art prior to being introduced into a cell (targeted for genome modification as described herein).

Plant cells differ from human and animal cells in that plant cells comprise a plant cell wall which can act as a barrier to the direct delivery of the RGEN ribonucleoproteins and/or of the direct delivery of the RGEN components.

Direct delivery of the RGEN ribonucleoproteins into plant cells can be achieved through particle mediated delivery (particle bombardment. Based on the experiments described herein, a skilled artesian can now envision that any other direct method of delivery, such as but not limiting to, polyethylene glycol (PEG)-mediated transfection to protoplasts, electroporation, cell-penetrating peptides, or mesoporous silica nanoparticle (MSN)-mediated direct protein delivery, can be successfully used for delivering RGEN ribonucleoproteins into plant cells.

Direct delivery of the RGEN ribonucleoprotein, allows for genome editing at a target site in the genome of a cell which can be followed by rapid degradation of the complex, and only a transient presence of the complex in the cell. This transient presence of the RGEN complex can lead to reduced off-target effects. In contrast, delivery of RGEN components (guide polynucleotide, Cas endonuclease) via plasmid DNA sequences can result in constant expression of RGENs from these plasmids which can intensify off target effects (Cradick, T. J. et al. (2013) Nucleic Acids Res 41:9584-9592; Fu, Y et al. (2014) Nat. Biotechnol. 31:822-826).

Direct delivery can be achieved by combining any one component of the guide polynucleotide/Cas endonuclease complex (RGEN) (such as at least one guide polynucleotide, at least one Cas protein, and at least one Cas protein), with a particle delivery matrix comprising a microparticle (such as but not limited to of a gold particle, tungsten particle, and silicon carbide whisker particle) (see also WO2017070032 published 27 Apr. 2017).

In some aspects, the guide polynucleotide/Cas endonuclease complex is a complex wherein the guide polynucleotide and Cas endonuclease protein forming the guide polynucleotide/Cas endonuclease complex are introduced into the cell as RNA and protein, respectively.

In some aspects, the guide polynucleotide/Cas endonuclease complex is a complex wherein the guide polynucleotide and Cas endonuclease protein and the at least one protein subunit of a Cas protein forming the guide polynucleotide/Cas endonuclease complex are introduced into the cell as RNA and proteins, respectively.

In some aspects, the guide polynucleotide/Cas endonuclease complex is a complex wherein the guide polynucleotide and Cas endonuclease protein and the at least one protein subunit of a Cascade forming the guide polynucleotide/Cas endonuclease complex (cleavage ready cascade) are preassembled in vitro and introduced into the cell as a ribonucleotide-protein complex.

Protocols for introducing polynucleotides, polypeptides or polynucleotide-protein complexes (PGEN, RGEN) into eukaryotic cells, such as plants or plant cells are known.

Alternatively, polynucleotides can be introduced into plant or plant cells by contacting cells or organisms with a virus or viral nucleic acids. Generally, such methods involve incorporating a polynucleotide within a viral DNA or RNA molecule. In some examples a polypeptide of interest can be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

The presently disclosed polynucleotides and polypeptides can be introduced into a cell. Cells include, but are not limited to, human, non-human, animal, mammalian, bacterial, protist, fungal, insect, yeast, non-conventional yeast, and plant cells, as well as plants and seeds produced by the methods described herein. In some aspects, the cell of the organism is a reproductive cell, a somatic cell, a meiotic cell, a mitotic cell, a stem cell, or a pluripotent stem cell.

### Cells and Plants

The presently disclosed polynucleotides and polypeptides can be introduced into a plant cell. Plant cells include, well as plants and seeds produced by the methods described herein. Any plant can be used with the compositions and methods described herein, including monocot and dicot plants, and plant elements.

The Cas endonucleases disclosed can be used to edit the genome of a plant cell in various ways. In some aspects, it can be desirable to delete one or more nucleotides. In another aspect, it can be desirable to insert one or more nucleotides. In some aspects, it can be desirable to replace one or more nucleotides. In another aspect, it can be desirable to modify one or more nucleotides via a covalent or non-covalent interaction with another atom or molecule. In some aspects, the cell is diploid. In some aspects, the cell is haploid.

Genome modification via a Cas endonuclease can be used to effect a genotypic and/or phenotypic change on the target organism. Such a change is preferably related to an improved trait of interest or an agronomically-important characteristic, the correction of an endogenous defect, or the expression of some type of expression marker. In some aspects, the trait of interest or agronomically-important characteristic is related to the overall health, fitness, or fertility of the plant, the yield of a plant product, the ecological fitness of the plant, or the environmental stability of the plant. In some aspects, the trait of interest or agronomically-important characteristic is selected from the group consisting of: agronomics, herbicide resistance, insecticide resistance, disease resistance, nematode resistance, microbial resistance, fungal resistance, viral resistance, fertility or sterility, grain characteristics, commercial product production. In some aspects, the trait of interest or agronomically-important characteristic is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered starch content, altered carbohydrate content, altered sugar content, altered fiber content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

Examples of monocot plants that can be used include, but are not limited to, corn (Zea mays), rice (Oryza sativa), rye (Secale cereale), Sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Additional plants that can be used include safflower (Carthamus tinctorius), sweet potato (Ipomoea batatus), cassava (Manihot esculenta), coffee (Coffea spp.), coconut (Cocos nucifera), Citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), Macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), Hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and Chrysanthemum.

Conifers that can be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In some aspects, a fertile plant is a plant that produces viable male and female gametes and is self-fertile. Such a self-fertile plant can produce a progeny plant without the contribution from any other plant of a gamete and the genetic material comprised therein. Other aspects of the disclosure can involve the use of a plant that is not self-fertile because the plant does not produce male gametes, or female gametes, or both, that are viable or otherwise capable of fertilization.

The present disclosure finds use in the breeding of plants comprising one or more edited alleles created by the methods or compositions disclosed herein. In some aspects, the edited alleles influence the phenotypic expression of one or more traits, such as plant health, growth, or yield. In some aspects, two plants can be crossed via sexual reproduction to create progeny plant(s) that comprise some or all of the edits from both parental plants.

While the disclosure has been particularly shown and described with reference to a preferred aspect and various alternate aspects, it will be understood by persons skilled in the relevant art that various changes in form and details can be made therein without departing from the spirit and scope of the disclosure. For instance, while the particular examples below can illustrate the methods and aspects described herein using a specific plant, the principles in these examples can be applied to any plant. Therefore, it will be appreciated that the scope of this disclosure is encompassed by the aspects of the disclosures recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific aspects of some aspects of the disclosure. The examples are offered for illustrative purposes only, and are not intended to limit the scope of the disclosure in any way. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

Although maize was used as a model, it will be understood by those of skill in the art that the methods and compositions described herein can be applied to any organism, particularly any plant, such as a monocot plant or a dicot plant.

Table 1 details sequences used in the Examples.

TABLE 1
SEQ ID Nos.

| SEQ | |
|---|---|
| ID | |
| NO. | Sequence |
| 1 | MDKKYSIGLDIGTNSVGWAVITDEYKVPSKKFKVLGNTDRHSIKKNLIGALLFDSGETAEATRLK |
| | RTARRRYTRRKNRICYLQEIFSNEMAKVDDSFFHRLEESFLVEEDKKHERHPIFGNIVDEVAYHE |
| | KYPTIYHLRKKLVDSTDKADLRLIYLALAHMIKFRGHFLIEGDLNPDNSDVDKLFIQLVQTYNQL |
| | FEENPINASGVDAKAILSARLSKSRRLENLIAQLPGEKKNGLFGNLIALSLGLTPNFKSNFDLAED |
| | AKLQLSKDTYDDDLDNLLAQIGDQYADLFLAAKNLSDAILLSDILRVNTEITKAPLSASMIKRYD |
| | EHHQDLTLLKALVRQQLPEKYKEIFFDQSKNGYAGYIDGGASQEEFYKFIKPILEKMDGTEELLV |
| | KLNREDLLRKQRTFDNGSIPHQIHLGELHAILRRQEDFYPFLKDNREKIEKILTFRIPYYVGPLARG |
| | NSRFAWMTRKSEETITPWNFEEVVDKGASAQSFIERMTNFDKNLPNEKVLPKHSLLYEYFTVYN |
| | ELTKVKYVTEGMRKPAFLSGEQKKAIVDLLFKTNRKVTVKQLKEDYFKKIECFDSVEISGVEDRF |
| | NASLGTYHDLLKIIKDKDFLDNEENEDILEDIVLTLTLFEDREMIEERLKTYAHLFDDKVMKQLK |
| | RRRYTGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGRHKPENIVIEMARENQTTQKGQKNSRERM |
| | KRIEEGIKELGSQILKEHPVENTQLQNEKLYLYYLQNGRDMYVDQELDINRLSDYDVDHIVPQSF |
| | LKDDSIDNKVLTRSDKNRGKSDNVPSEEVVKKMKNYWRQLLNAKLITQRKFDNLTKAERGGLS |
| | ELDKAGFIKRQLVETRQITKHVAQILDSRMNTKYDENDKLIREVKVITLKSKLVSDFRKDFQFYK |
| | VREINNYHHAHDAYLNAVVGTALIKKYPKLESEFVYGDYKVYDVRKMIAKSEQEIGKATAKYF |
| | FYSNIMNFFKTEITLANGEIRKRPLIETNGETGEIVWDKGRDFATVRKVLSMPQVNIVKKTEVQT |
| | GGFSKESILPKRNSDKLIARKKDWDPKKYGGFDSPTVAYSVLVVAKVEKGKSKKLKSVKELLGI |
| | TIMERSSFEKNPIDFLEAKGYKEVKKDLIIKLPKYSLFELENGRKRMLASAGELQKGNELALPSKY |
| | VNFLYLASHYEKLKGSPEDNEQKQLFVEQHKHYLDEIIEQISEFSKRVILADANLDKVLSAYNKH |
| | RDKPIREQAENIIHLFTLTNLGAPAAFKYFDTTIDRKRYTSTKEVLDATLIHQSITGLYETRIDLSQL |
| | GGD |
| 2 | MGESVKAIKLKILDMFLDPECTKQDDNWRKDLSTMSRFCAEAGNMCLRDLYNYFSMPKEDRIS |
| | SKDLYNAMYHKTKLLHPELPGKVANQIVNHAKDVWKRNAKLIYRNQISMPTYKITTAPIRLQNN |
| | IYKLIKNKNKYIIDVQLYSKEYSKDSGKGTHRYFLVAVRDSSTRMIFDRIMSKDHIDSSKSYTQGQ |
| | LQIKKDHQGKWYCIIPYTFPTHETVLDPDKVMGVDLGVAKAVYWAFNSSYKRGCIDGGEIEHFR |
| | KMIRARRVSIQNQIKHSGDARKGHGRKRALKPIETLSEKEKNFRDTINHRYANRIVEAAIKQGCG |
| | TIQIENLEGIADTTGSKFLKNWPYYDLQTKIVNKAKEHGITVVAINPQYTSQRCSMCGYIEKTNRS |
| | SQAVFECKQCGYGSRTICINCRHVQVSGDVCEECGGIVKKENVNADYNAAKNISTPYIDQIIMEK |
| | CLELGIPYRSITCKECGHIQASGNTCEVCGSTNILKPKKIRKAK |
| 3 | MGESVKAIKLKILDMFLDPECTKQDDNWRKDLSTMSRFCGEAGNMCLRDLYNYFSMPKEDRIS |
| | SKDLYNAMYHKTKLLHPGLPGKVKNQIVNHAKDVWKRNAKLIYRNQISMPTYKITTAPIRLQN |
| | NIYKLIKNKNKYIIDVQLYSKEYSKDSGKGTHRYFLVAVRDSSTRMIFDRIMSKDHIDSSKSYKQ |
| | GQLQIKKDHQGKWYCIIPYTFPTHEHVLDPDKVMGVDLGVAKAVYWAFNSSYKRGCIDGGEIE |
| | HFRKMIRARRVSIQNQIKHSGDARKGHGRKRALKPIETLSEKESNFRDTINFRYANRIVEAAIKQG |
| | CGTIQIENLEGIADRTGSKFLKNWPYYDLQTKIVNKAKEHGITVVAINPQYTSQRCSMCGYIEKT |
| | NRSSQAVFECKQCGYGSRTNCINCRHVQVSGDVCEECGGIVKKENVNADYNAAKNISTPYIDQII |
| | MEKCLELGIPYRSITCKECGHIQASGNTCEVCGSTNILKPKKIRKAK |
| 4 | MGESVKAIKLKILDMFLDPECTKQDDNWRKDLSTMSRFCGEAGNSCLRDLYNYDSMPKEDRISS |
| | KDLYNAMYHKTKLLHPELPGQVKNQIVNHAKDVWKRNAKLIYRNQISMPTYKITTAPIRLONNI |
| | YKLIKNKNKYIIDVQLYSKEYSKDSGKGTHRYFLVAVRDSSTRMIFDRIMSKDHIDSSKSYKQGQ |
| | LQIKKDHQGKWYCIIPYTFPTHEHVLDPDKVMGVDLGVAKAVYWAFNSSYKRGCIDGGEIEHFR |
| | KMIRARRVSIQNQIKHSGDARKGHGRKRALKPIETLSEKESNFRDTINFRYANRIVEAAIKQGCGT |
| | IQIENLEGIADRTGSKFLKNWPYYDLQTKIVNKAKEHGITVVAINPQYTSQRCSMCGYIEKTNRSS |
| | QAVFECKQCGYGSRTNCINCRHVQVSGDVCEECGGIVKKENVNADYNAAKNISTPYIDQIIMEK |
| | CLELGIPYRSITCKECDHIQASGNTCEVCGSTNILKPKKIRKAK |
| 5 | MIKVYRYEIVKPLDLDWKEFGTILRQLQQETRFALNKATQLAWEWMGFSSDYKDNHGEYPKSK |
| | DILGYTNVHGYAYHTIKTKAYRLNSGNLSQTIKRATDRFKAYQKEILRGDMSIPSYKRDIPLDLIK |
| | ENISVNRMNHGDYIASLSLLSNPAKQEMNVKRKISVIIIVRGAGKTIMDRILSGEYQVSASQIIHDD |
| | RKNKWYLNISYDFEPQTRVLDLNKIMGIDLGVAVAVYMAFQHTPARYKLEGGEIENFRRQVESR |
| | RISMLRQGKYAGGARGGHGRDKRIKPIEQLRDKIANFRDTTNHRYSRYIVDMAIKEGCGTIQME |
| | DLTNIRDIGSRFLQNWTYYDLQQKIIYKAEEAGIKVIKIDPQYTSQRCSECGNIDSGNRIGQAIFKC |
| | RACGYEANADYNAARNIAIPNIDKIIAESIK |
| 6 | MIKVYRYEIVKPLDLDWKEFGTILRQLQQETRFALNKATQLAWEWMGFSSDYKDNHGEYPKSK |
| | DILGYTNVHGYAYHTIKTKAYRLNSGNLSQTIKRATDRFKAYQKEILRGDMSIPSYKRDYPLDLI |
| | KENISVNRMNHGDYIASLSLLSNPAKQEMNVKRKISVIIIVRGAGKTIMDRILSGEYQVSASQIIHD |
| | DRKNKWYLNISYDFEPQTRVLDLNKIMGIDLGVAVAVYMAFQHTPARYKLEGGEIENFRRQVES |
| | RRISMLTQGKYAGGARGGHGRDKRIKPIEQLRDKIPNFRDTTNHRYSRYIVDMAIKEGCGTIQME |
| | DLTNIRDIGSRFLQNWTYYDLQQKIIYKAEEAGIKVIKIDPQYTSQRCSECGNIDSGNRIGQAIFKC |
| | RACGYEANADYNAARNIAIPNIDKIIAESIK |
| 7 | KRPRDRHDGELGGRKRAR |
| 8 | GTGCAGCGTGACCCGGTCGTGCCCCTCTCTAGAGATAATGAGCATTGCATGTCTAAGTTATA |
| | AAAAATTACCACATATTTTTTTTGTCACACTTGTTTGAAGTGCAGTTTATCTATCTTTATACAT |
| | ATATTTAAACTTTACTCTACGAATAATATAATCTATAGTACTACAATAATATCAGTGTTTTAG |
| | AGAATCATATAAATGAACAGTTAGACATGGTCTAAAGGACAATTGAGTATTTTGACAACAG |
| | GACTCTACAGTTTTATCTTTTTAGTGTGCATGTGTTCTCCTTTTTTTTTGCAAATAGCTTCACC |
| | TATATAATACTTCATCCATTTTATTAGTACATCCATTTAGGGTTTAGGGTTAATGGTTTTTATA |
| | GACTAATTTTTTTAGTACATCTATTTTATTCTATTTTAGCCTCTAAATTAAGAAAACTAAAAC |
| | TCTATTTTAGTTTTTTTATTTAATAATTTAGATATAAAATAGAATAAAATAAAGTGACTAAAA |
| | ATTAAACAAATACCCTTTAAGAAATTAAAAAAACTAAGGAAACATTTTTCTTGTTTCGAGTA |
| | GATAATGCCAGCCTGTTAAACGCCGTCGACGAGTCTAACGGACACCAACCAGCGAACCAGC |
| | AGCGTCGCGTCGGGCCAAGCGAAGCAGACGGCACGGCATCTCTGTCGCTGCCTCTGGACCCC |
| | TCTCGAGAGTTCCGCTCCACCGTTGGACTTGCTCCGCTGTCGGCATCCAGAAATTGCGTGGC |
| | GGAGCGGCAGACGTGAGCCGGCACGGCAGGCGGCCTCCTCCTCCTCTCACGGCACCGGCAG |
| | CTACGGGGGATTCCTTTCCCACCGCTCCTTCGCTTTCCCTTCCTCGCCCGCCGTAATAAATAG |
| | ACACCCCCTCCACACCCTCTT |
| 9 | TCCCCAACCTCGTGTTGTTCGGAGCGCACACACACACAACCAGATCTCCCCCAAATCCACCC |
| | GTCGGCACCTCCGCTTCAAG |
| 10 | GTACGCCGCTCGTCCTCCCCCCCCCCCCTCTCTACCTTCTCTAGATCGGCGTTCCGGTCCATG |
| | CATGGTTAGGGCCCGGTAGTTCTACTTCTGTTCATGTTTGTGTTAGATCCGTGTTTGTGTTAG |
| | ATCCGTGCTGCTAGCGTTCGTACACGGATGCGACCTGTACGTCAGACACGTTCTGATTGCTA |
| | ACTTGCCAGTGTTTCTCTTTGGGGAATCCTGGGATGGCTCTAGCCGTTCCGCAGACGGGATC |
| | GATTTCATGATTTTTTTTGTTTCGTTGCATAGGGTTTGGTTTGCCCTTTTCCTTTATTTCAATAT |
| | ATGCCGTGCACTTGTTTGTCGGGTCATCTTTTCATGCTTTTTTTTGTCTTGGTTGTGATGATGT |
| | GGTCTGGTTGGGCGGTCGTTCTAGATCGGAGTAGAATTCTGTTTCAAACTACCTGGTGGATTT |
| | ATTAATTTTGGATCTGTATGTGTGTGCCATACATATTCATAGTTACGAATTGAAGATGATGGA |
| | TGGAAATATCGATCTAGGATAGGTATACATGTTGATGCGGGTTTTACTGATGCATATACAGA |
| | GATGCTTTTTGTTCGCTTGGTTGTGATGATGTGGTGTGGTTGGGCGGTCGTTCATTCGTTCTA |
| | GATCGGAGTAGAATACTGTTTCAAACTACCTGGTGTATTTATTAATTTTGGAACTGTATGTGT |
| | GTGTCATACATCTTCATAGTTACGAGTTTAAGATGGATGGAAATATCGATCTAGGATAGGTA |
| | TACATGTTGATGTGGGTTTTACTGATGCATATACATGATGGCATATGCAGCATCTATTCATAT |
| | GCTCTAACCTTGAGTACCTATCTATTATAATAAACAAGTATGTTTTATAATTATTTTGATCTT |
| | GATATACTTGGATGATGGCATATGCAGCAGCTATATGTGGATTTTTTTAGCCCTGCCTTCATA |
| | CGCTATTTATTTGCTTGGTACTGTTTCTTTTGTCGATGCTCACCCTGTTGTTTGGTGTTACTTC |
| | TGCAG |
| 11 | ATGGGCGAGAGCGTCAAGGCAATAAAATTAAAGATCCTCGACATGTTCCTCGACCCCGAGT |
| | GCACCAAGCAGGTAAGTTTCTGCTTCTACCTTTGATATATATATAATAATTATCATTAATTAG |
| | TAGTAATATAATATTTCAAATATTTTTTTCAAAATAAAAGAATGTAGTATATAGCAATTGCTT |
| | TTCTGTAGTTTATAAGTGTGTATATTTTAATTTATAACTTTTCTAATATATGACCAAAACATG |
| | GTGATGTGCAGGACGACAACTGGCGCAAGGACCTCAGCACCATGAGCCGCTTCTGCGGCGA |
| | GGCCGGCAACATGTGCCTCAGGGACCTCTACAACTACTTCAGCATGCCCAAGGAGGACCGC |
| | ATCAGCTCCAAGGACTTATATAACGCCATGTACCATAAAACTAAGCTCCTCCACCCCGGCCT |
| | CCCCGGGAAGGTGAAGAACCAAATCGTCAACCACGCCAAGGACGTCTGGAAGCGCAACGCC |
| | AAGCTCATCTACCGCAACCAAATCAGCATGCCCACATATAAGATCACCACCGCCCCCATCCG |
| | CCTCCAAAATAACATCTACAAATTAATAAAAAATAAGAACAAATACATAATCGACGTCCAG |
| | CTCTACAGCAAGGAGTACAGCAAGGACAGCGGCAAGGGCACCCACAGGTACTTCCTCGTCG |
| | CCGTCAGGGACAGCAGCACCAGGATGATCTTCGACAGGATCATGAGCAAGGACCACATCGA |
| | CAGCAGCAAGAGCTACAAGCAGGGCCAGCTCCAAATCAAGAAGGACCACCAGGGCAAGTG |
| | GTACTGCATCATCCCCTATACATTCCCCACCCACGAACACGTCCTCGACCCCGACAAGGTCA |
| | TGGGCGTCGACCTCGGGGTGGCTAAGGCCGTCTACTGGGCTTTCAACAGCAGCTATAAAAGA |
| | GGCTGCATCGACGGCGGCGAGATCGAGCACTTCAGGAAGATGATCAGGGCCCGGCGCGTCA |
| | GCATCCAGAATCAAATCAAACACAGCGGCGACGCCCGCAAGGGCCACGGCAGGAAGAGGG |
| | CCCTCAAGCCCATCGAAACCCTCAGCGAGAAGGAGAGCAACTTCCGCGACACAATAAACTT |
| | CAGGTACGCCAACAGGATCGTCGAGGCCGCTATCAAGCAGGGCTGCGGCACCATCCAGATC |
| | GAGAACCTCGAGGGCATCGCTGACAGGACCGGCAGCAAGTTCCTCAAGAACTGGCCCTACT |
| | ACGACCTCCAGACCAAGATCGTCAATAAAGCCAAGGAGCACGGCATCACCGTCGTCGCAAT |
| | AAACCCCCAGTATACATCCCAGCGCTGCAGCATGTGCGGCTACATCGAGAAAACCAACAGG |
| | AGCAGCCAGGCCGTGTTCGAGTGCAAGCAGTGCGGCTACGGCAGCCGCACCAACTGCATCA |
| | ACTGCAGGCACGTCCAAGTCTCCGGCGACGTCTGCGAGGAGTGCGGCGGCATCGTCAAGAA |
| | GGAGAACGTCAACGCCGACTACAACGCCGCCAAGAACATCAGCACCCCCTACATCGACCAG |
| | ATAATAATGGAGAAGTGCCTCGAGCTCGGCATCCCCTACCGCTCCATCACCTGCAAGGAGTG |
| | CGGCCACATCCAGGCTAGCGGCAACACCTGCGAGGTCTGCGGCAGCACCAACATCCTCAAA |
| | CCAAAGAAGATCCGCAAGGCAAAA |
| 12 | ATGGGCGAGAGCGTCAAGGCAATAAAATTAAAGATCCTCGACATGTTCCTCGACCCCGAGT |
| | GCACCAAGCAGGACGACAACTGGCGCAAGGACCTCAGCACCATGAGCCGCTTCTGCGGCGA |
| | GGCCGGCAACATGTGCCTCAGGGACCTCTACAACTACTTCAGCATGCCCAAGGAGGACCGC |
| | ATCAGCTCCAAGGACTTATATAACGCCATGTACCATAAAACTAAGCTCCTCCACCCCGGCCT |
| | CCCCGGGAAGGTGAAGAACCAAATCGTCAACCACGCCAAGGACGTCTGGAAGCGCAACGCC |
| | AAGCTCATCTACCGCAACCAAATCAGCATGCCCACATATAAGATCACCACCGCCCCCATCCG |
| | CCTCCAAAATAACATCTACAAATTAATAAAAAATAAGAACAAATACATAATCGACGTCCAG |
| | CTCTACAGCAAGGAGTACAGCAAGGACAGCGGCAAGGGCACCCACAGGTACTTCCTCGTCG |
| | CCGTCAGGGACAGCAGCACCAGGATGATCTTCGACAGGATCATGAGCAAGGACCACATCGA |
| | CAGCAGCAAGAGCTACAAGCAGGGCCAGCTCCAAATCAAGAAGGACCACCAGGGCAAGTG |
| | GTACTGCATCATCCCCTATACATTCCCCACCCACGAACACGTCCTCGACCCCGACAAGGTCA |
| | TGGGCGTCGACCTCGGGGTGGCTAAGGCCGTCTACTGGGCTTTCAACAGCAGCTATAAAAGA |
| | GGCTGCATCGACGGCGGCGAGATCGAGCACTTCAGGAAGATGATCAGGGCCCGGCGCGTCA |
| | GCATCCAGAATCAAATCAAACACAGCGGCGACGCCCGCAAGGGCCACGGCAGGAAGAGGG |
| | CCCTCAAGCCCATCGAAACCCTCAGCGAGAAGGAGAGCAACTTCCGCGACACAATAAACTT |
| | CAGGTACGCCAACAGGATCGTCGAGGCCGCTATCAAGCAGGGCTGCGGCACCATCCAGATC |
| | GAGAACCTCGAGGGCATCGCTGACAGGACCGGCAGCAAGTTCCTCAAGAACTGGCCCTACT |
| | ACGACCTCCAGACCAAGATCGTCAATAAAGCCAAGGAGCACGGCATCACCGTCGTCGCAAT |
| | AAACCCCCAGTATACATCCCAGCGCTGCAGCATGTGCGGCTACATCGAGAAAACCAACAGG |
| | AGCAGCCAGGCCGTGTTCGAGTGCAAGCAGTGCGGCTACGGCAGCCGCACCAACTGCATCA |
| | ACTGCAGGCACGTCCAAGTCTCCGGCGACGTCTGCGAGGAGTGCGGCGGCATCGTCAAGAA |
| | GGAGAACGTCAACGCCGACTACAACGCCGCCAAGAACATCAGCACCCCCTACATCGACCAG |
| | ATAATAATGGAGAAGTGCCTCGAGCTCGGCATCCCCTACCGCTCCATCACCTGCAAGGAGTG |
| | CGGCCACATCCAGGCTAGCGGCAACACCTGCGAGGTCTGCGGCAGCACCAACATCCTCAAA |
| | CCAAAGAAGATCCGCAAGGCAAAA |
| 13 | GTAAGTTTCTGCTTCTACCTTTGATATATATATAATAATTATCATTAATTAGTAGTAATATAA |
| | TATTTCAAATATTTTTTTCAAAATAAAAGAATGTAGTATATAGCAATTGCTTTTCTGTAGTTT |
| | ATAAGTGTGTATATTTTAATTTATAACTTTTCTAATATATGACCAAAACATGGTGATGTGCAG |
| 14 | CCGAAGAAGAAGCGCAAGGTG |
| 15 | GTCATGGGTCGTTTAAGCTGCCGATGTGCCTGCGTCGTCTGGTGCCCTCTCTCCATATGGAGG |
| | TTGTCAAAGTATCTGCTGTTCGTGTCATGAGTCGTGTCAGTGTTGGTTTAATAATGGACCGGT |
| | TGTGTTGTGTGTGCGTACTACCCAGAACTATGACAAATCATGAATAAGTTTGATGTTTGAAA |
| | TTAAAGCCTGTGCTCATTATGTTCTGTCTTTCAGTTGTCTCCTAATATTTGCCTGCAGGTACTG |
| | GCTATCTACCGTTTCTTACTTAGGAGGTGTTTGAATGCACTAAAACTAATAGTTAGTGGCTAA |
| | AATTAGTTAAAACATCCAAACACCATAGCTAATAGTTGAACTATTAGCTATTTTTGGAAAAT |
| | TAGTTAATAGTGAGGTAGTTATTTGTTAGCTAGCTAATTCAACTAACAATTTTTAGCCAACTA |
| | ACAATTAGTTTCAGTGCATTCAAACACCCCCTTAATGTTAACGTGGTTCTATCTACCGTCTCC |
| | TAATATATGGTTGATTGTTCGGTTTGTTGCTATGCTATTGGGTTCTGATTGCTGCTAGTTCTTG |
| | CTGAATCCAGAAGTTCTCGTAGTATAGCTCAGATTCATATTATTTATTTGAGTGATAAGTGAT |
| | CCAGGTTATTACTATGTTAGCTAGGTTTTTTTTACAAGGATAAATTATCTGTGATCATAATTC |
| | TTATGAAAGCTTTATGTTTCCTGGAGGCAGTGGCATGCAATGCATGACAGCAACTTGATCAC |
| | ACCAGCTGAGGTAGATACGGTAACAAGGTTCTTAAATCTGTTCACCAAATCATTGGAGAACA |
| | CACATACACATTCTTGCCAGTCTTGGTTAGAGAAATTTCATGACAAAATGCCAAAGCTGTCT |
| | TGACTCTTCACTTTTGGCCATGAGTCGTGAC |
| 16 | TGAGAGTACAATGATGAACCTAGATTAATCAATGCCAAAGTCTGAAAAATGCACCCTCAGTC |
| | TATGATCCAGAAAATCAAGATTGCTTGAGGCCCTGTTCGGTTGTTCCGGATTAGAGCCCCGG |
| | ATTAATTCCTAGCCGGATTACTTCTCTAATTTATATAGATTTTGATGAGCTGGAATGAATCCT |
| | GGCTTATTCCGGTACAACCGAACAGGCCCTGAAGGATACCAGTAATCGCTGAGCTAAATTGG |
| | CATGCTGTCAGAGTGTCAGTATTGCAGCAAGGTAGTGAGATAACCGGCATCATGGTGCCAGT |
| | TTGATGGCACCATTAGGGTTAGAGATGGTGGCCATGGGCGCATGTCCTGGCCAACTTTGTAT |
| | GATATATGGCAGGGTGAATAGGAAAGTAAAATTGTATTGTAAAAAGGGATTTCTTCTGTTTG |
| | TTAGCGCATGTACAAGGAATGCAAGTTTTGAGCGAGGGGGCATCAAAGATCTGGCTGTGTTT |
| | CCAGCTGTTTTTGTTAGCCCCATCGAATCCTTGACATAATGATCCCGCTTAAATAAGCAACCT |
| | CGCTTGTATAGTTCCTTGTGCTCTAACACACGATGATGATAAGTCGTAAAATAGTGGTGTCC |
| | AAAGAATTTCCAGGCCCAGTTGTAAAAGCTAAAATGCTATTCGAATTTCTACTAGCAGTAAG |
| | TCGTGTTTAGAAATTATTTTTTTATATACCTTTTTTCCTTCTATGTACAGTAGGACACAGTGTC |
| | AGCGCCGCGTTGACGGAGAATATTTGCAAAAAAGTAAAAGAGAAAGTCATAGCGGCGTATG |
| | TGCCAAAAACTTCGTCACAGAGAGGGCCATAAGAAACATGGCCCACGGCCCAATACGAAGC |
| | ACCGCGACGAAGCCCAAACAGCAGTCCGTAGGTGGAGCAAAGCGCTGGGTAATACGCAAAC |
| | GTTTTGTCCCACCTTGACTAATCACAAGAGTGGAGCGTACCTTATAAACCGAGCCGCAAGCA |
| | CCGAATTG |
| 17 | AACAAAGCACCAGTGGTCTAGTGGTAGAATAGTACCCTGCCACGGTACAGACCCGGGTTCG |
| | ATTCCCGGCTGGTGCA |
| 18 | ACAAAAGCACCAGTGGTCTAGTGGTAGAATAGTACCCTGCCACGGTACAGACCCGGGTTCG |
| | ATTCCCGGCTGGTGCA |
| 19 | ATTTACTCTGTTTCGCGCGCCAGGGCAGTTAGGTGCCCTAAAAGAGCGAAGTGGCCGAAAG |
| | GAAAGGCTAACGCTTCTCTAACGCTACGGCGACCTTGGCGAAATGCCATCAATACCACGCGG |
| | CCCGAAAGGGTTCGCGCGAAACTGAGTAATGAAAGTCGCATCTTGCGTAAGCGCGTGGATT |
| | GAAAC |
| 20 | AATGCCGCAGTTCATTGAGC |
| 21 | TCATGTCTACTGGTCTATTT |
| 22 | AGTTCAGAGAAGGCAACCTT |
| 23 | CACAGGGAGGGAGCAAACAA |
| 24 | AAGTTCACGGCGTTCCAGGC |
| 25 | GGCCGGCATGGTCCCAGCCTCCTCGCTGGCGCCGGCTGGGCAACATGCTTCGGCATGGCGAA |
| | TGGGAC |
| 26 | TTTTTTTT |
| 27 | MELEIRPLFLVPDTNGFIDHLASLARLLESRKYILVVPLIVINELDGLAKGQETDHRAGGYARVVQ |
| | EKARKSIEFLEQRFESRDSCLRALTSRGNELESIAFRSEDITGQLGNNDDLILSCCLHYCKDKAKD |
| | FMPASKEEPIRLLREVVLLTDDRNLRVKALTRNVPVRDIPAFLTWAQVG |
| 28 | MASNFTQFVLVDNGGTGDVTVAPSNFANGVAEWISSNSRSQAYKVTCSVRQSSAQKRKYTIKV |
| | EVPKVATQTVGGVELPVAAWRSYLNMELTIPIFATNSDCELIVKAMQGLLKDGNPIPSAIAANSG |
| | IY |
| 29 | MSKTIVLSVGEATRTLTEIQSTADRQIFEEKVGPLVGRLRLTASLRQNGAKTAYRVNLKLDQADV |
| | VDCSTSVCGELPKVRYTQVWSHDVTIVANSTEASRKSLYDLTKSLVATSQVEDLVVNLVPLGR |
| 30 | SAGGGGSGGGGSGGGGSGPKKKRKVAAAGS |
| 31 | KRPRDRHDGELGGRKRARGRA |
| 32 | KRPRDRHDGELGGRKRARGGSGGSGGS |
| 33 | KRPRDRHDGELGGRKRARGRAVDTGNGS |
| 34 | SRADPKKKRKVGRA |
| 35 | SRADPKKKRKVGGSGGSGGS |
| 36 | SRADPKKKRKVDTGNGS |
| 37 | ACATGAGGATCACCCATGT |
| 38 | TAAGGAGTTTATATGGAAACCCTTA |
| 39 | CTACAATA |
| 40 | CCCTAGCTCCCTGCGGCTGTTACGCGGTCCCCCATCAATCTTCTGTTCTTGCGGTTGTAGCCT |
| | GTGTAACAGTGCTAGAGTATGTATGATAAATAGGTTTTAAGTCTGCTTACATGACATTTTTTA |
| | TTGTGGAAGAGACATATAAAAATTAGAGAGAGTGGTTCTCATGCAACGGCGGACGGCCCGG |
| | TGCTAAAAGAGCTTCAAGACAAAATAATGAAACAGGAAGAGAGTAGATTTATCTAAGAGCC |
| | AACTTTATTATATGAATGTGTTTATTGTTGGCTTTAGATGATATGGTAAGGAGTTAGAGCTAA |
| | TAATAGATAGGCTCTATTATTATTATTATTAATTAAACTCGCTCTAAGGAGGAAAGTGGGAG |
| | GAAGGGACGAGGACGAAGACTACTGGAAGCATCGTGCATGGATGATGGATGTGGTGTCTCT |
| | TAATGTAGGTGGCCGGAGGATGTACGTGTTAATTGCGCGATAAGCACTCAGATCCAACCGCA |
| | AACTACCTCCACACTGACACACTGATAGAGAGAAAGAGAGACCTCCGACGACTGCCGCCGC |
| | AGATGAGCCACGTACGTATACGACGTCTGCCGGCCGGCTCAGGCTGCCGCCATCACCCTGCT |
| | CGAAAGTCGCGTTAGGCGGCGCCAGCTACATAGGAGTATCTAGTCTAGCCAGTTAGTATACT |
| | ACTACTGCGCTGATGATGAATTAACTCTGCATAGATACTGTACTTGCCTCCCTCCAACACCCA |
| | ACCACCTCCTGCTCGGCTCTTAATAACTTGGACACGGATCGATGCCATCCAAGGAAGAACAC |
| | GACGACGACGACGGAACATCCACCATGCAAGCTTGCATCCATACGCCGATACGCGTGCATC |
| | CATCCATCCACCATTATTTCCATTTTCCACCGATCACACGTACACAGGCCTATTTAAGGAGCG |
| | ACATCCCACTGCAACTCTCCTCACCACTCATCACCAGCTAG |
| 41 | CTCTAGCAAAGCACTTGCCATCTACCGACCGCCGCATTCCAAACAGCCCGACGAGCTAGCAG |
| | AGCGGCAGGCACCTCCCTCCTCAAGGAAC |
| 42 | GTAAATCTGATGAGTCCGTGAGGACGAAACGAGTAAGCTCGTC |
| 43 | AGGATTATTCCG |
| 44 | ATGGGCTCAAAAGTAGCAGGTAAAAAGAAAACCCAGAATGATAATAAACTAGATAACGAA |
| | AATGGTTCACAGCAGCGCGAAAATATCAATACCAAGACGCTTTTGAAAGGCAACCTTAAGA |
| | TATCAAATTACAAATATCTTGAGGTGATTCAACTAGAACATGCTGTGACAAAATTGGTGGAG |
| | TCTTACAATAAAATAATTGAACTTTCACCAAATTTGGTAGCTTACAATGAAGCCGTTAACAA |
| | TCAAGATAGAGTGCCTGTTCAGATACTTCCTTCTCTATCACGTTATCAATTAAAACTGGCAGC |
| | TGAATTAAAAACCTTACATGATCTTAAAAAAGACGCAATTTTGACAGAAATTACTGATTATG |
| | AAAATGAATTTGATACTGAACAAAAGCAACCTATATTGCAAGAAATCAGTAAAGCTGATAT |
| | GGAAAAGTTAGAGAAGCTAGAACAAGTTAAAAGGGAAAAGCGAGAGAAAATAGATGTAAA |
| | TGTGTACGAAAATTTAAATGAAAAGGAAGATGAAGAAGAAGACGAAGGAGAAGATAGCTA |
| | TGACCCAACAAAGGCTGGTGATATCGTCAAGGCAACAAAATGGCCTCCAAAATTACCAGAG |
| | ATTCAAGATTTAGCGATTAGGGCCAGGGTATTTATTCACAAATCCACAATAAAGGATAAAGT |
| | TTACTTGTCTGGATCAGAAATGATCAACGCACATAATGAAAGACTAGAATTCCTAGGCGATT |
| | CGATCTTAAATTCTGTTATGACATTAATTATTTATAACAAGTTTCCCGATTACAGCGAGGGTC |
| | AGTTATCAACATTAAGGATGAATTTGGTAAGCAACGAACAGATCAAACAATGGTCAATAAT |
| | GTACAATTTCCATGAGAAACTTAAGACAAATTTTGACTTGAAGGATGAAAATTCCAATTTTC |
| | AAAACGGCAAACTAAAACTGTATGCAGATGTGTTCGAAGCTTACATTGGCGGCTTGATGGA |
| | AGATGATCCAAGGAATAATTTGCCCAAAATAAGAAAATGGTTAAGAAAGCTTGCCAAACCT |
| | GTCATTGAGGAGGCTACCCGTAATCAAGTTGCCTTGGAGAAGACGGATAAACTTGATATGA |
| | ATGCTAAAAGGCAGCTTTACTCTTTGATTGGCTATGCTTCATTACGTCTACATTATGTTACCG |
| | TAAAAAAACCCACTGCAGTTGATCCTAATTCCATAGTTGAGTGCAGAGTAGGTGATGGAACA |
| | GTTTTAGGGACCGGTGTGGGCAGAAATATCAAAATTGCGGGTATTAGGGCTGCAGAAAATG |
| | CTCTTCGTGACAAAAAAATGTTAGATTTTTACGCCAAACAAAGAGCTGCCATTCCTAGGAGT |
| | GAATCTGTGTTAAAAGATCCCTCACAAAAGAATAAGAAAAGAAAATTCTCAGATACAAGCT |
| | GA |
| 45 | MGSKVAGKKKTQNDNKLDNENGSQQRENINTKTLLKGNLKISNYKYLEVIQLEHAVTKLVESY |
| | NKIIELSPNLVAYNEAVNNQDRVPVQILPSLSRYQLKLAAELKTLHDLKKDAILTEITDYENEFDT |
| | EQKQPILQEISKADMEKLEKLEQVKREKREKIDVNVYENLNEKEDEEEDEGEDSYDPTKAGDIV |
| | KATKWPPKLPEIQDLAIRARVFIHKSTIKDKVYLSGSEMINAHNERLEFLGDSILNSVMTLIIYNKF |
| | PDYSEGQLSTLRMNLVSNEQIKQWSIMYNFHEKLKTNFDLKDENSNFQNGKLKLYADVFEAYIG |
| | GLMEDDPRNNLPKIRKWLRKLAKPVIEEATRNQVALEKTDKLDMNAKRQLYSLIGYASLRLHY |
| | VTVKKPTAVDPNSIVECRVGDGTVLGTGVGRNIKIAGIRAAENALRDKKMLDFYAKQRAAIPRS |
| | ESVLKDPSQKNKKRKFSDTS |
| 46 | ATGGGCAGCAAGGTGGCCGGCAAGAAGAAGACCCAGAACGACAACAAGCTCGACAACGAG |
| | AACGGCAGCCAGCAGAGGGAGAACATCAACACCAAGACCCTCCTCAAGGGCAACCTCAAGA |
| | TCAGCAACTACAAGTACCTCGAGGTGATCCAGCTCGAGCACGCCGTGACCAAGCTCGTGGA |
| | GAGCTACAACAAGATCATCGAGCTCAGCCCGAACCTCGTGGCCTACAACGAGGCCGTGAAC |
| | AACCAGGACAGGGTGCCGGTGCAGATCCTCCCGAGCCTCAGCAGGTACCAGCTCAAGCTCG |
| | CCGCCGAGCTCAAGACCCTCCACGACCTCAAGAAGGACGCCATCCTCACCGAGATCACCGA |
| | CTACGAGAACGAGTTCGACACCGAGCAGAAGCAGCCGATCCTCCAGGAGATCAGCAAGGCC |
| | GACATGGAGAAGCTCGAGAAGCTCGAGCAGGTGAAGAGGGAGAAGAGGGAGAAGATCGAC |
| | GTGAACGTGTACGAGAACCTCAACGAGAAGGAGGACGAGGAGGAGGACGAGGGCGAGGAC |
| | AGCTACGACCCGACCAAGGCCGGCGACATCGTGAAGGCCACCAAGTGGCCGCCGAAGCTCC |
| | CGGAGATCCAGGACCTCGCCATCAGGGCCAGGGTGTTCATCCACAAGAGCACCATCAAGGA |
| | CAAGGTGTACCTCAGCGGCAGCGAGATGATCAACGCCCACAACGAGAGGCTCGAGTTCCTC |
| | GGCGACAGCATCCTCAACAGCGTGATGACCCTCATCATCTACAACAAGTTCCCGGACTACAG |
| | CGAGGGCCAGCTCAGCACCCTCAGGATGAACCTCGTGAGCAACGAGCAGATCAAGCAGTGG |
| | AGCATCATGTACAACTTCCACGAGAAGCTCAAGACCAACTTCGACCTCAAGGACGAGAACA |
| | GCAACTTCCAGAACGGCAAGCTCAAGCTCTACGCCGACGTGTTCGAGGCCTACATCGGCGGC |
| | CTCATGGAGGACGACCCGAGGAACAACCTCCCGAAGATCAGGAAGTGGCTCAGGAAGCTCG |
| | CCAAGCCGGTGATCGAGGAGGCCACCAGGAACCAGGTGGCCCTCGAGAAGACCGACAAGCT |
| | CGACATGAACGCCAAGAGGCAGCTCTACAGCCTCATCGGCTACGCCAGCCTCAGGCTCCACT |
| | ACGTGACCGTGAAGAAGCCGACCGCCGTGGACCCGAACAGCATCGTGGAGTGCAGGGTGGG |
| | CGACGGCACCGTGCTCGGCACCGGCGTGGGCAGGAACATCAAGATCGCCGGCATCAGGGCC |
| | GCCGAGAACGCCCTCAGGGACAAGAAGATGCTCGACTTCTACGCCAAGCAGAGGGCCGCCA |
| | TCCCGAGGAGCGAGAGCGTGCTCAAGGACCCGAGCCAGAAGAACAAGAAGAGGAAGTTCA |
| | GCGACACCAGCTAG |
| 47 | GGCGUUUAGAUGUCAUGUCAUGAGUCCAUGGCAUGGCAGUUAGACGCC |
| 48 | GGCGCUAUCGUGUCAUGUCAUGAGUCCAUGGCAUGGCAUGAUAGCGCC |
| 49 | UUUCCUUUGACUUCGCAUCAUGAAUGAGUUCAUUCAUGAUGAAGCGAAAGGAGAGA |
| 50 | CUUUUCUACAAAUUUGAUUCAUGAGUCCAUGGAACAAAUAUAUAGAACUC |
| 51 | ACUGGGAUAUAUGUGAGUUCACGUGCGUCCCAAU |
| 52 | UUUCUUUCUAAGUGGGUACUGGCAGGAGUCGGGGCCUAGUUUAGAGAGAAG |
| 53 | GGCGTTTAGATGTCATGTCATGAGTCCATGGCATGGCAGTTAGACGCC |
| 54 | GGCGCTATCGTGTCATGTCATGAGTCCATGGCATGGCATGATAGCGCC |
| 55 | TTTCCTTTGACTTCGCATCATGAATGAGTTCATTCATGATGAAGCGAAAGGAGAGA |
| 56 | CTTTTCTACAAATTTGATTCATGAGTCCATGGAACAAATATATAGAACTC |
| 57 | ACTGGGATATATGTGAGTTCACGTGCGTCCCAAT |
| 58 | TTTCTTTCTAAGTGGGTACTGGCAGGAGTCGGGGCCTAGTTTAGAGAGAAG |
| 59 | ATGTGCCAATCGCGGGGGGTTTTCGTTCAGGACTACAACTGCCACACACCACCAAAGCTAAC |
| | TGACAGGAGAATCCAGATGGATGCACAAACACGCCGCCGCGAACGTCGCGCAGAGAAACA |
| | GGCTCAATGGAAAGCAGCAAATCCCCTGTTGGTTGGGGTAAGCGCAAAACCAGTTAACCGC |
| | CCTATTCTCTCGCTGAATCGCAAACCGAAATCACGAGTAGAAAGCGCACTAAATCCGATAGA |
| | CCTTACAGTGCTGGCTGAATACCACAAACAGATTGAAAGCAACCTGCAACGTATTGAGCGC |
| | AAGAATCAGCGCACATGGTACAGCAAGCCTGGCGAACGCGGCATAACATGCAGTGGACGCC |
| | AGAAAATTAAGGGAAAATCGATTCCTCTTATCTAG |
| 60 | MCQSRGVFVQDYNCHTPPKLTDRRIQMDAQTRRRERRAEKQAQWKAANPLLVGVSAKPVNRPI |
| | LSLNRKPKSRVESALNPIDLTVLAEYHKQIESNLQRIERKNQRTWYSKPGERGITCSGRQKIKGKS |
| | IPLI |
| 61 | ATGGATGCACAAACACGCCGCCGCGAACGTCGCGCAGAGAAACAGGCTCAATGGAAAGCA |
| | GCAAAT |
| 62 | MDAQTRRRERRAEKQAQWKAAN |
| 63 | ATGACGGTTATCACCTACGGGAAGTCAACGTTTGCAGGCAATGCTAAAACTCGCCGTCATGA |
| | GCGGCGCAGAAAGCTAGCCATAGAGCGCGACACCATCTGCAATATCATCGATTCAATTTTTG |
| | GCTGCGATGCTCCTGATGCTTCTCAGGAAGTTAAAGCCAAAAGAATTGACCGTGTCACCAAA |
| | GCCATTTCGCTTGCCGGAACGCGTCAGAAGGAAGTTGAAGGAGGATCTGTACTTCTTCCAGG |
| | CGTAGCACTTTACGCGGCTGGTCATCGTAAGAGCAAACAAATAACAGCGAGGTAA |
| 64 | MTVITYGKSTFAGNAKTRRHERRRKLAIERDTICNIIDSIFGCDAPDASQEVKAKRIDRVTKAISLA |
| | GTRQKEVEGGSVLLPGVALYAAGHRKSKQITAR |
| 65 | GGCAATGCTAAAACTCGCCGTCATGAGCGGCGCAGAAAGCTAGCCATAGAGCGCGACACCA |
| | TC |
| 66 | GNAKTRRHERRRKLAIERDTI |
| 67 | GATGTCATGTCATGAGTCCATGGCATGGCAGTTA |
| 68 | CGTGTCATGTCATGAGTCCATGGCATGGCATGAT |
| 69 | ATTGACGGTACGGTACCTGAGTACTGTACTGTAG |
| 70 | TAGTACGGTACGGGACCTGAGTACTGTACTGTGC |
| 71 | ATGGGCAGCAAGGTGGCCGGCAAGAAGAAGACCCAGAACGACAACAAGCTCGACAACGAG |
| | AACGGCAGCCAGCAGAGGGAGAACATCAACACCAAGACCCTCCTCAAGGGCAACCTCAAGA |
| | TCAGCAACTACAAGTACCTCGAGGTGATCCAGCTCGAGCACGCCGTGACCAAGCTCGTGGA |
| | GAGCTACAACAAGATCATCGAGCTCAGCCCGAACCTCGTGGCCTACAACGAGGCCGTGAAC |
| | AACCAGGACAGGGTGCCGGTGCAGATCCTCCCGAGCCTCAGCAGGTATCAGCTCAAGCTCG |
| | CCGCCGAGCTCAAGACCCTCCACGACCTCAAGAAGGACGCCATCCTCACCGAGATCACCGA |
| | CTACGAGAACGAGTTCGACACCGAGCAGAAGCAGCCGATCCTCCAGGAGATCAGCAAGGCC |
| | GACATGGAGAAGCTCGAGAAGCTCGAGCAGGTGAAGAGGGAGAAGAGGGAGAAGATCGAC |
| | GTGAACGTGTACGAGAACCTCAACGAGAAGGAGGACGAGGAGGAGGACGAGGGCGAGGAC |
| | AGCTACGACCCGACCAAGGCCGGCGACATCGTGAAGGCCACCAAGTGGCCGCCGAAGCTCC |
| | CGGAGATCCAGGACCTCGCCATCAGGGCCAGGGTGTTCATCCACAAGAGCACCATCAAGGA |
| | CAAGGTGTACCTCAGCGGCAGCGAGATGATCAACGCCCACAACGAGAGGCTCGAGTTCCTC |
| | GGCGACAGCATCCTCAACAGCGTGATGACCCTCATCATCTACAACAAGTTCCCGGACTACAG |
| | CGAGGGCCAGCTCAGCACCCTCAGGATGAACCTCGTGAGCAACGAGCAGATCAAGCAGTGG |
| | AGCATCATGTACAACTTCCACGAGAAGCTCAAGACCAACTTCGACCTCAAGGACGAGAACA |
| | GCAACTTCCAGAACGGCAAGCTCAAGCTCTACGCCGACGTGTTCGAGGCCTACATCGGCGGC |
| | CTCATGGAGGACGACCCGAGGAACAACCTCCCGAAGATCAGGAAGTGGCTCAGGAAGCTCG |
| | CCAAGCCGGTGATCGAGGAGGCCACCAGGAACCAGGTGGCCCTCGAGAAGACCGACAAGCT |
| | CGACATGAACGCCAAGAGGCAGCTCTACAGCCTCATCGGCTACGCCAGCCTCAGGCTCCACT |
| | ACGTGACCGTGAAGAAGCCGACCGCCGTGGACCCGAACAGCATCGTGGAGTGCAGGGTGGG |
| | CGACGGCACCGTGCTCGGCACCGGCGTGGGCAGGAACATCAAGATCGCCGGCATCAGGGCC |
| | GCCGAGAACGCCCTCAGGGACAAGAAGATGCTCGACTTCTACGCCAAGCAGAGGGCCGCCA |
| | TCCCGAGGAGCGAGAGCGTGCTCAAGGACCCGAGCCAGAAGAACAAGAAGAGGAAGTTCA |
| | GCGACACCAGCTAG |
| 72 | MGSKVAGKKKTQNDNKLDNENGSQQRENINTKTLLKGNLKISNYKYLEVIQLEHAVTKLVESY |
| | NKIIELSPNLVAYNEAVNNQDRVPVQILPSLSRYQLKLAAELKTLHDLKKDAILTEITDYENEFDT |
| | EQKQPILQEISKADMEKLEKLEQVKREKREKIDVNVYENLNEKEDEEEDEGEDSYDPTKAGDIV |
| | KATKWPPKLPEIQDLAIRARVFIHKSTIKDKVYLSGSEMINAHNERLEFLGDSILNSVMTLIIYNKF |
| | PDYSEGQLSTLRMNLVSNEQIKQWSIMYNFHEKLKTNFDLKDENSNFQNGKLKLYADVFEAYIG |
| | GLMEDDPRNNLPKIRKWLRKLAKPVIEEATRNQVALEKTDKLDMNAKRQLYSLIGYASLRLHY |
| | VTVKKPTAVDPNSIVECRVGDGTVLGTGVGRNIKIAGIRAAENALRDKKMLDFYAKQRAAIPRS |
| | ESVLKDPSQKNKKRKFSDTS |
| 73 | ATGGGCAGCAAGGTGGCCGGCAAGAAGAAGACCCAGAACGACAACAAGCTCGACAACGAG |
| | AACGGCAGCCAGCAGAGGGAGAACATCAACACCAAGACCCTCCTCAAGGGCAACCTCAAGA |
| | TCAGCAACTACAAGTACCTCGAGGTGATCCAGCTCGAGCACGCCGTGACCAAGCTCGTGGA |
| | GAGCTACAACAAGATCATCGAGCTCAGCCCGAACCTCGTGGCCTACAACGAGGCCGTGAAC |
| | AACCAGGACAGGGTGCCGGTGCAGATCCTCCCGAGCCTCAGCAGGTATCAGCTCAAGCTCG |
| | CCGCCGAGCTCAAGACCCTCCACGACCTCAAGAAGGACGCCATCCTCACCGAGATCACCGA |
| | CTACGAGAACGAGTTCGACACCGAGCAGAAGCAGCCGATCCTCCAGGAGATCAGCAAGGCC |
| | GACATGGAGAAGCTCGAGAAGCTCGAGCAGGTAAGTTTCTGCTTCTACCTTTGATATATATA |
| | TAATAATTATCATTAATTAGTAGTAATATAATATTTCAAATATTTTTTTCAAAATAAAAGAAT |
| | GTAGTATATAGCAATTGCTTTTCTGTAGTTTATAAGTGTGTATATTTTAATTTATAACTTTTCT |
| | AATATATGACCAAAACATGGTGATGTGCAGGTGAAGAGGGAGAAGAGGGAGAAGATCGAC |
| | GTGAACGTGTACGAGAACCTCAACGAGAAGGAGGACGAGGAGGAGGACGAGGGCGAGGAC |
| | AGCTACGACCCGACCAAGGCCGGCGACATCGTGAAGGCCACCAAGTGGCCGCCGAAGCTCC |
| | CGGAGATCCAGGACCTCGCCATCAGGGCCAGGGTGTTCATCCACAAGAGCACCATCAAGGA |
| | CAAGGTGTACCTCAGCGGCAGCGAGATGATCAACGCCCACAACGAGAGGCTCGAGTTCCTC |
| | GGCGACAGCATCCTCAACAGCGTGATGACCCTCATCATCTACAACAAGTTCCCGGACTACAG |
| | CGAGGGCCAGCTCAGCACCCTCAGGATGAACCTCGTGAGCAACGAGCAGATCAAGCAGTGG |
| | AGCATCATGTACAACTTCCACGAGAAGCTCAAGACCAACTTCGACCTCAAGGACGAGAACA |
| | GCAACTTCCAGAACGGCAAGCTCAAGCTCTACGCCGACGTGTTCGAGGCCTACATCGGCGGC |
| | CTCATGGAGGACGACCCGAGGAACAACCTCCCGAAGATCAGGAAGTGGCTCAGGAAGCTCG |
| | CCAAGCCGGTGATCGAGGAGGCCACCAGGAACCAGGTGGCCCTCGAGAAGACCGACAAGCT |
| | CGACATGAACGCCAAGAGGCAGCTCTACAGCCTCATCGGCTACGCCAGCCTCAGGCTCCACT |
| | ACGTGACCGTGAAGAAGCCGACCGCCGTGGACCCGAACAGCATCGTGGAGTGCAGGGTGGG |
| | CGACGGCACCGTGCTCGGCACCGGCGTGGGCAGGAACATCAAGATCGCCGGCATCAGGGCC |
| | GCCGAGAACGCCCTCAGGGACAAGAAGATGCTCGACTTCTACGCCAAGCAGAGGGCCGCCA |
| | TCCCGAGGAGCGAGAGCGTGCTCAAGGACCCGAGCCAGAAGAACAAGAAGAGGAAGTTCA |
| | GCGACACCAGCTAG |
| 74 | GGGTAATACGCAAACGTTTTGTCCCACCTTGACTAATCACAAGAGTGGAGCGTACCTTATAA |
| | ACCGAGCCGCAAGCACCGAATTG |

### Example 1: CRISPR-Associated Proteins for Plant Expression and Nuclear Localization

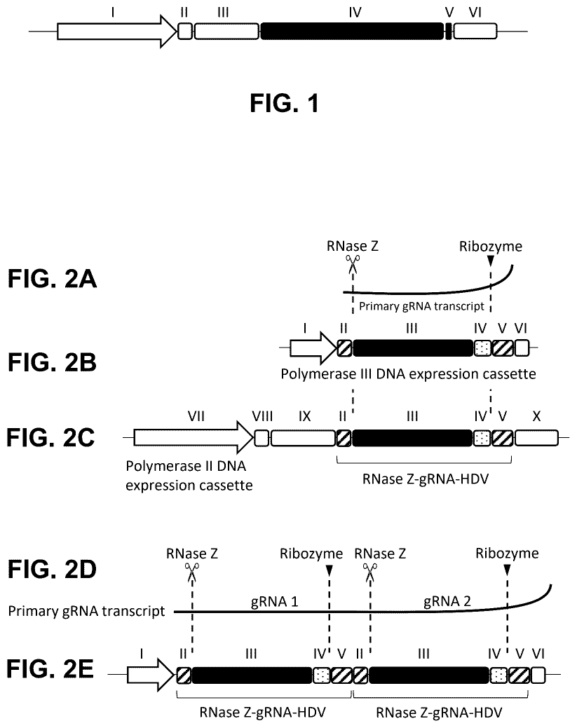

To confer efficient expression in maize cells, the Cas polypeptide (SEQ ID NOs: 1-6) was constructed using routine methods. To facilitate nuclear localization of the Cas polypeptide in maize cells, a nucleotide sequence encoding a nuclear localization signal (NLS) was added to either or both ends of the Cas polynucleotide sequence encoding the Cas polypeptide. Exemplary nuclear localization signals include, but are not limited to, a monopartite Simian virus 40 (SV40) NLS (PKKKRKV) and a bi-partite NLS from the VirD2 protein from Agrobacterium tumefaciens (SEQ ID NO:7). The nucleotide sequence encoding the Cas polynucleotide and NLS, were then operably concatenated with a Zea mays Ubiquitin promoter (ZM-UBI PRO), Zea mays Ubiquitin 5′ untranslated region (ZM-UBI UTR), Zea mays Ubiquitin intron 1 (ZM-UBI INTRON 1), and Zea mays Ubiquitin terminator (ZM-UBI TERM), synthesized, and then cloned into a DNA plasmid using restriction enzyme digestion and ligation (GenScript, USA). The resulting Cas expression cassettes are illustrated in FIG. 1.

### Example 2: Transformation of Optimized Cas Expression Cassettes

In this example, methods for introducing a Cas polynucleotide and guide RNA expression cassette into a eukaryotic cell, particularly a plant cell, are described.

Suitable transformation methods for the methods disclosed herein include, for example, Agrobacterium-mediated delivery, Ensifer-based delivery, nanoparticle-mediated delivery, and particle-mediated biolistic delivery, as well as approaches utilizing protoplasts (Sardesai and Subramanyam (2018) Agrobacterium Biology: From Basic Science to Biotechnology. Cham: Springer International Publishing, 463-488, Rathore et al. (2019) Transgenic Plants: Methods and Protocols. New York, NY: Springer New York, 37-48, Wang et al. (2019) Molecular Plant. 12, 1037-1040, Rhodes et al. (1988) Science. 240, 204-207 and Golovkin et al. (1993) Plant Science. 90, 41-52). In the present Example, Cas endonuclease and guide RNA plasmid expression cassettes were co-delivered into 9-10 day old maize immature embryos using particle-mediated biolistic transformation (Svitashev et al. (2015) Plant Physiology. 169, 931-945 and Karvelis et al. (2015) Genome Biology. 16, 253). Briefly, DNA expression cassettes were co-precipitated onto 0.6 μM (average size) gold particles utilizing TransIT-2020. Next, the DNA-coated gold particles were pelleted by centrifugation, washed with absolute ethanol, and re-dispersed by sonication. Following sonication, 10 μl of the DNA-coated gold particles were loaded onto a macrocarrier and air dried. Next, biolistic transformation was performed using a PDS-1000/He Gun (Bio-Rad) with a 425 pound per square inch rupture disc. To promote cell division, BabyBoom (BBM) and Wuschel2 (WUS2) genes expressed from non-constitutive promoters, maize phospholipid transferase protein, and maize auxin-inducible, respectively, were also co-delivered alongside the Cas endonuclease and guide RNA expression cassettes (Lowe et al. (2018) In vitro cellular & developmental biology-Plant. 54, 240-252). A visual marker DNA expression cassette encoding a fluorescent protein, for example, a yellow fluorescent protein, can also be co-delivered to aid in the selection of evenly transformed tissue. Moreover, a chemical selectable marker (for example but not limited to neomycin phosphotransferase II) can also be delivered with the aforementioned plasmids to select for transformed cells. To foster culture conditions optimal for Cas protein activity, transformed tissue can be incubated at 28° C., standard for particle gun transformation, or at a range of temperatures lower or higher than 28° C.

### Example 3: Analysis of Target Sites for Cellular DNA Editing

In this example, methods for analyzing genomic DNA target sites for evidence of cleavage and cellular repair are described.

Transient experiments were performed similar to that described in Svitashev et al., 2015, Karvelis et al., 2015, and Bigelyte et al. (2021) Nature Communications. 12, 6191. In a first method, transformed immature maize embryos were harvested 2-10 days after transformation, genomic DNA extracted, and Cas targets examined by Ampli-seq for the presence of mutations indicative of RNA-guided Cas editing as described previously (Svitashev et al., 2015 and Bigelyte et al., 2021). Briefly, the 20-30 most evenly transformed immature embryos, based on their fluorescence, were harvested for each experiment. Next, total genomic DNA was extracted and the region surrounding the intended target site was PCR amplified with Phusion® HighFidelity PCR Master Mix (New England Biolabs, M0531L) adding on the sequences necessary for amplicon-specific barcodes and Illumina sequencing using “tailed” primers through two rounds of PCR and deep sequenced. The resulting reads were then examined for the presence of mutations at the expected site of cleavage by comparison to control experiments where the small RNA expression cassette was omitted from the transformation. Sequence reads containing putative indels were further validated as true mutations by confirming their absence in the control datasets.

In a second method, regenerated plants are sampled, and DNA targets examined for evidence of editing using Ampli-Seq. This is performed as described above, although, to assess the likelihood of inheritance, the frequency of edited and wildtype sequence reads for each plant is also calculated. Since maize is diploid, plants with ˜50% and ˜100% mutant reads can be assumed to be heterozygous and homozygous, respectively, for the targeted mutation (Zhang et al., (2014) Plant Biotechnology Journal. 12, 797-807).

### Example 4: Guide RNA Transcript Processing

In this Example, methods for expressing one or more guide RNA (gRNA) species as a single transcript that is processed to produce a mature gRNA or multiple gRNAs capable of directing one or more Cas proteins to a DNA target site(s) in a eukaryotic cell, particularly a plant cell, are described.

In a first method, both an RNase Z and a self-cleaving RNA sequence, a ribozyme, were used to excise one or more gRNAs from a primary RNA transcript in a cell (FIGS. 2A-2E). For this, a sequence encoding a RNase Z recognition site containing promoter elements capable of recruiting RNA polymerase III (Schiffer et al. (2002) The EMBO Journal. 21, 2769-2777, White et al. (2011) Nature Reviews Genetics. 12, 459-463, and Dieci et al. (2007) Trends in Genetics. 23, 614-622) and a ribozyme from the Hepatitis Delta Virus (HDV) (Peng et al. (2021) RSC Chemical Biology. 2, 1370-1383) were appended to the 5′ and 3′ ends, respectively, of the DNA sequence encoding each gRNA. The resulting sequence was then operably inserted into a polymerase II or III expression cassette comprising a promoter and terminator, for example but not limited to, a polymerase III U6 promoter and terminator, or a polymerase II Zea mays Ubiquitin promoter and terminator (ZM-UBI). When using the Zea mays Ubiquitin promoter, a Zea mays Ubiquitin 5′ UTR and Zea mays Ubiquitin intron 1 were also incorporated between the promoter and RNase Z recognition site. To enable expression of multiple gRNAs from a single promoter, sequences encoding two or more gRNAs flanked with a 5′ RNase Z and 3′ ribozyme were first concatenated and then linked with the respective promoter and terminator. The RNase Z-gRNA-HDV expression cassettes were then synthesized and cloned into a plasmid DNA (GenScript, USA).

Examples of RNase Z-gRNA-HDV expression cassettes are shown in FIGS. 2A-2E. FIGS. 2B and 2C illustrate a polymerase III DNA expression cassette and a polymerase II expression cassette, respectively, each comprising a DNA sequence encoding a guide RNA flanked by a DNA sequence encoding an RNase Z recognition site and a DNA sequence encoding an HDV ribozyme (RNase Z-gRNA-HDV). FIG. 2A illustrates excision of a guide RNA from a primary transcript via RNase Z (scissors represent an RNase Z cleavage site) and the self-cleaving HDV ribozyme (triangle represents the ribozyme self-cleavage site). FIG. 2E illustrates a DNA expression cassette comprising a first DNA sequence encoding a first guide RNA and a second DNA sequence encoding a second guide RNA, wherein each DNA sequence encoding a guide RNA is flanked by a DNA sequence encoding an RNase Z recognition site and a DNA sequence encoding an HDV ribozyme. FIG. 2D illustrates excision of a first and a second guide RNA from a primary transcript via RNase Z (scissors represent an RNase Z cleavage site) and the self-cleaving HDV ribozyme (triangles represent the ribozyme self-cleavage site).

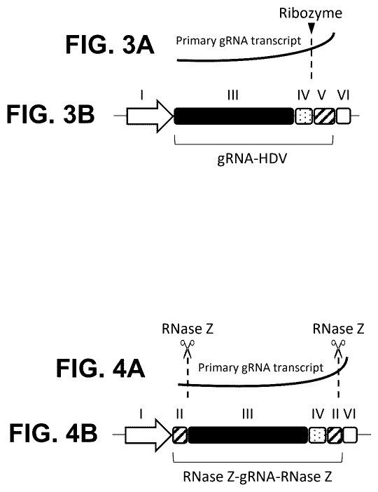

Initially, U6 RNase Z-gRNA-HDV constructs encoding one gRNA were tested. As comparators, other gRNA expression cassette designs, gRNA-HDV and RNase Z-gRNA-RNase Z, were also included (FIGS. 3A-3B and 4A-4B).

Examples of gRNA-HDV and RNase Z-gRNA-RNase Z expression cassettes are shown in FIGS. 3A-4B. FIG. 3B illustrates a DNA expression cassette comprising a DNA sequence encoding a guide RNA flanked by a 3′ DNA sequence encoding an HDV ribozyme. FIG. 3A illustrates excision of a guide RNA from a primary transcript via the self-cleaving HDV ribozyme (triangle represents the ribozyme self-cleavage site). FIG. 4B illustrates a DNA expression cassette comprising a DNA sequence encoding a guide RNA flanked by 5′ and 3′ DNA sequences encoding RNase Z recognition sites. FIG. 4A illustrates excision of a guide RNA from a primary transcript via RNase Z (scissors represent RNase Z cleavage sites).

As shown in Table 2, the RNase Z-gRNA-HDV design out-performed the RNase Z-gRNA-RNase Z cassette and yielded editing frequencies similar to the gRNA-HDV construct under all conditions tested. Table 2: MS1 target site mutagenesis frequencies using 30 ng, 10 ng, or 5 ng of the respective guide RNA expression cassette in combination with 80 ng of the Cas expression construct in transient experiments that were placed at 37° C. and harvested three days after particle gun transformation. As a negative control, the guide RNA expression plasmid was omitted. Two repetitions of each transformation condition were performed.

TABLE 2
Target site mutagenesis using a combination of a RNaseZ recognition
site and a HDV ribozyme for guide RNA maturation

| gRNA | | Average | |
|---|---|---|---|
| Concen- | | Percentage | |
| tration | | of Mutant | Standard |
| (ng) | gRNA Expression Cassette | Reads | Deviation |
| 0 | no gRNA | 0.01 | 0.01 |
| 30 | 227b_MS1* | 0.49 | 0.25 |
| | S_PIII_rT_G_rT_227b_MS1 | 0.22 | 0.09 |
| | S_PIII_rT_G_H_227b_MS1 | 0.35 | 0.18 |
| | S_PIII_mT_G_mT_227b_MS1 | 0.30 | 0.20 |
| | S_PIII_mT_G_H_227b_MS1* | 0.45 | 0.06 |
| 10 | 227b_MS1* | 0.27 | 0.08 |
| | S_PIII_rT_G_rT_227b_MS1 | 0.10 | 0.02 |
| | S_PIII_rT_G_H_227b_MS1 | 0.23 | 0.08 |
| | S_PIII_mT_G_mT_227b_MS1 | 0.11 | 0.04 |
| | S_PIII_mT_G_H_227b_MS1* | 0.24 | 0.07 |
| 5 | 227b_MS1 | 0.15 | 0.06 |
| | S_PIII_rT_G_rT_227b_MS1 | 0.07 | 0.04 |
| | S_PIII_rT_G_H_227b_MS1* | 0.17 | 0.04 |
| | S_PIII_mT_G_mT_227b_MS1 | 0.10 | 0.10 |
| | S_PIII_mT_G_H_227b_MS1* | 0.24 | 0.08 |

227b_MS1=U6 promoter-gRNA-HDV, S_PIII_rT_G_rT_227b_MS1=U6 promoter-rice RNase Z-gRNA-rice RNase Z, S_PIII_rT_G_H_227b_MS1=U6 promoter-rice RNase Z-gRNA-HDV, S_PIII_mT_G_mT_227b_MS1=U6 promoter-maize RNase Z-gRNA-maize RNase Z, S_PIII_mT_G_H_227b_MS1=U6 promoter-maize RNase Z-gRNA-HDV

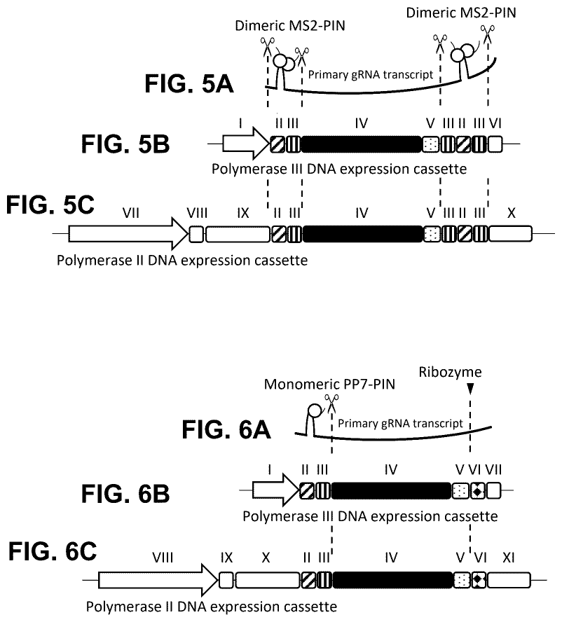

In another method, a bacteriophage coat protein recognition sequence and a bacteriophage coat protein engineered to contain a PilT N-terminus (PIN) ribonuclease domain are used to excise one or more gRNAs from a primary RNA transcript in a cell (FIGS. 5A-5C). For this, the PIN ribonuclease domain (SEQ ID NO:27) from the human telomerase-binding protein EST1A is first fused to a N56K version of the MS2 bacteriophage coat protein (Lim et al. (1994) Nucleic Acids Research. 22, 3748-3752) (SEQ ID NO:28) or the coat protein from the Pseudomonas phage, PP7 (SEQ ID NO:29) with a protein linker containing a VirD2 or SV40 NLS (SEQ ID NO:30-36). Sequences encoding the viral coat protein PIN ribonuclease fusion are next optimized for expression in maize as described above, operably concatenated with a constitutive promoter (e.g., Zea mays Ubiquitin gene and associated 5′UTR and intron (SEQ ID NO:8-10)) or a non-constitutive promoter (e.g., Zea mays phospholipid transferase protein (PLTP) gene promoter and associated 5′ UTR (SEQ ID NO:40-41)), synthesized, and cloned into a DNA plasmid using restriction enzyme digestion and ligation (GenScript, USA). For use with MS2-PIN fusions, the 5′ and 3′ ends of a sequence encoding one or more gRNAs are flanked with sequences encoding a stem-loop forming (sf) RNA (SEQ ID NO:37) capable of complexing with two copies of the MS2 coat protein, placed in the context of a polymerase II or III expression cassette, synthesized, and cloned into a DNA plasmid as described above. When using the PP7-PIN design, the 5′ end of a sequence encoding one or more gRNAs is flanked with a sequence encoding a sfRNA (SEQ ID NO: 38) that can be recognized by a single copy of the PP7 coat protein while the 3′ end of the gRNA is linked to a sequence encoding a HDV ribozyme (SEQ ID NO:25), and operably placed into a polymerase II or III expression cassette as described above. To establish optimal spacing for PIN ribonuclease cleavage, sequences encoding a linker (li) RNA of various lengths (e.g, SEQ ID NO: 39) were optionally incorporated between the sfRNA and gRNA. Examples of the viral coat protein linked PIN ribonuclease gRNA expression cassettes are shown in FIGS. 5A-5C and FIGS. 6A-6C.

FIGS. 5A-5C illustrate another method for excising one or more guide RNAs from a primary transcript. FIG. 5B illustrates a polymerase III DNA expression cassette comprising a DNA sequence encoding a guide RNA flanked by 5′ and 3′ DNA sequences encoding MS2 coat protein recognition sequences in the form of stem loop-forming RNA. FIG. 5C illustrates a polymerase II DNA expression cassette comprising a DNA sequence encoding a guide RNA flanked by 5′ and 3′ DNA sequences encoding MS2 coat protein recognition sequences in the form of stem loop-forming RNA. FIG. 5A illustrates excision of a guide RNA from a primary transcript via the MS2 coat protein recognition sequences of the primary sequence complexing with two MS2-PIN fusion proteins (circles represent the MS2 coat protein and scissors represent the PIN ribonuclease linked domain).

FIGS. 6A-6C illustrate another method for excising one or more guide RNAs from a primary transcript. FIG. 6B illustrates a polymerase III DNA expression cassette comprising a DNA sequence encoding a guide RNA flanked by DNA sequences encoding a PP7 coat protein recognition sequence in the form of stem loop-forming RNA at its 5′ end and a self-cleaving HDV ribozyme at its 3′ end. FIG. 6C illustrates a polymerase II DNA expression cassette comprising a DNA sequence encoding a guide RNA flanked DNA sequences encoding a PP7 coat protein recognition sequence in the form of stem loop-forming RNA at its 5′ end and a self-cleaving HDV ribozyme at its 3′ end. FIG. 6A illustrates excision of a guide RNA from a primary transcript via the PP7 coat protein fused to the PIN ribonuclease and a HDV ribozyme (circles represent the PP7 coat protein, scissors represent the PIN ribonuclease linked domain, and the triangle represents the ribozyme self-cleavage site).

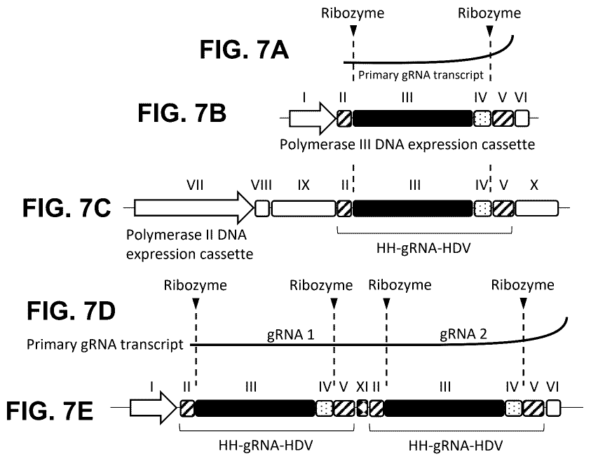

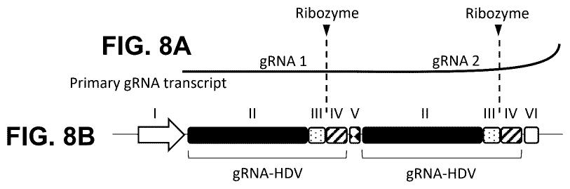

In another method, one or more self-cleaving viral ribozymes are used to excise one or more gRNAs from a primary transcript in a cell. For this, sequences encoding a Hammerhead (HH) ribozyme (SEQ ID NO:42) (Scott et al. (1996) Science. 274, 2065-2069) and a HDV ribozyme (SEQ ID NO:25) (Nakano et al. (2000) Science. 287, 1493-1497) are attached to the 5′ and 3′ ends of a sequence encoding a gRNA or a sequence encoding only a HDV ribozyme is appended to the 3′ end of a sequence encoding a gRNA. To facilitate proper ribozyme folding and self-cleavage, a sequence encoding a flexible 5′-CUUG-3′ tetraloop (Hall et al. (2013) PNAS USA. 110, 16706-16707) or conserved gRNA nexus-like sequence from the most abundant family of Cas9 trans-activating RNAs (SEQ ID NO:43) (Dooley et al. (2021) The CRISPR Journal. 4, 438-447) can optionally be inserted between HH-gRNA-HDV or gRNA-HDV encoding sequences. Then, as detailed above, the resulting sequences are linked with promoter and terminator elements, synthesized, and cloned into a DNA plasmid. Examples of HH-gRNA-HDV and gRNA-HDV expression cassettes are illustrated in FIGS. 7A-7E and FIGS. 8A-8B.

FIGS. 7A-7C illustrate another method for excising one or more guide RNAs from a primary transcript. FIGS. 7D-7E illustrate another method for excising one or more guide RNAs from a primary transcript. FIG. 7B illustrates a polymerase III DNA expression cassette comprising a DNA sequence encoding a guide RNA flanked by a 5′ DNA sequence encoding an HH ribozyme, and a 3′ DNA sequence encoding an HDV ribozyme. FIG. 7C illustrates a polymerase II DNA expression cassette comprising a DNA sequence encoding a guide RNA flanked by a 5′ DNA sequence encoding an HH ribozyme and a 3′ DNA sequence encoding an HDV ribozyme. FIG. 7A illustrates excision of a guide RNA from a primary transcript via the HH and HDV self-cleaving ribozymes (triangles represent the ribozyme self-cleavage site). FIG. 7E illustrates a polymerase III DNA expression cassette comprising a first DNA sequence encoding a first guide RNA and a second DNA sequence encoding a second guide RNA, wherein each DNA sequence encoding a guide RNA is flanked by a 5′ DNA sequence encoding an HH ribozyme and a 3′ DNA sequence encoding an HDV ribozyme. FIG. 7D illustrates excision of a first and a second guide RNA from a primary transcript via the self-cleaving HH and HDV ribozymes (triangles represent the ribozyme self-cleavage site).

FIGS. 8A and 8B illustrate another method for excising one or more guide RNAs from a primary transcript. FIG. 8B illustrates a DNA expression cassette comprising a first DNA sequence encoding a first guide RNA and a second DNA sequence encoding a second guide RNA, wherein the 3′ end of the first and second guide RNAs are flanked by a DNA sequence encoding an HDV ribozyme. FIG. 8A illustrates excision of a first and a second guide RNA from a primary transcript via the self-cleaving HDV ribozymes (triangles represent the ribozyme self-cleavage site).

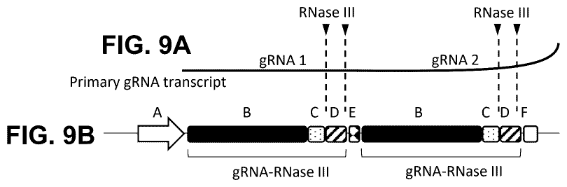

In yet another method, RNase III and RNase III recognition sequence were used to excise one or more gRNAs from a primary RNA transcript in a cell. Here, a gene (SEQ ID NO: 71) encoding the Saccharomyces cerevisiae Rnt1 protein (a RNase III enzyme) (SEQ ID NO: 72) was expressed behind a maize Ubiquitin promoter, 5′ UTR, and intron although regulatable or tissue specific expression promoting elements could also be used (for example but not limited to the PLTP promoter and associated 5′ UTR (SEQ ID NOs: 40 and 41)). To eliminate its expression in E. coli, an intron (SEQ ID NO:13) was also optionally inserted into the maize conditioned rnt1 gene resulting in SEQ ID NO: 73. A maize Ubiquitin promoter, 5′ UTR, and intron or other expression elements was used to drive expression of multiple gRNA sequences, each flanked by a RNase III stem-loop recognition sequence containing the consensus ATNN loop sequence. Examples of efficiently recognized and cleaved stem-loop structures include sequences C8 and C13 from Babiskin and Smolke (2011, Mol. Sys. Biol. 7:471). In such an example using a single repetitive stem loop such as C8 interspersed in between each of the gRNA sequences, the expressed Rnt1 protein acts as a homodimer with one molecule of the enzyme binding each side of the stem loop and facilitating cleavage of both sides of the stem immediately adjacent to the gRNA sequences. FIGS. 9A and 9B illustrates cleavage of the stem-loop which releases the individual gRNA sequences. FIG. 9B illustrates a DNA expression cassette comprising a first DNA sequence encoding a first guide RNA and a second DNA sequence encoding a second guide RNA, wherein the 3′ end of the first and second guide RNAs are flanked by a DNA sequence encoding an RNase III stem-loop recognition sequence. FIG. 9A illustrates excision of a first and a second guide RNA from a primary transcript via RNase III (triangles represent cleavage sites).

As shown in Table 3, the C13 Rnt1 recognition sequenced resulted in in average targeted indel frequencies around 4-fold above background (transformations where the guide RNA expression cassette was omitted or expression cassettes that contained the Rnt1 recognition sequence in a reverse orientation, C08r and C13r). Table 3: WX4, WUS2, MS1, CR82, and CR36 target site mutagenesis frequencies using the respective guide RNA expression cassette in combination with 80 ng of the Cas expression construct in transient experiments that were placed at 37° C. and harvested three days after particle gun transformation. As a negative control, the guide RNA expression plasmid was omitted. Three repetitions of each transformation condition were performed. The Zea mays Ubiquitin promoter, 5′ UTR, and intron in addition to a partial U6 promoter (SEQ ID NO: 74) were used to drive expression of one gRNA.

TABLE 3
Target site mutagenesis using Rnt1-mediated
guide RNA maturation (one guide RNA)

| | | Average | |
|---|---|---|---|
| | | Percentage | |
| Target | | of Mutant | Standard |
| Site | gRNA Expression Cassette | Reads | Deviation |
| WX4 | no gRNA | 0.11 | 0.01 |
| WUS2 | no gRNA | 0.08 | 0.02 |
| MS1 | no gRNA | 0.00 | 0.00 |
| CR82 | no gRNA | 0.00 | 0.00 |
| CR36 | no gRNA | 0.00 | 0.01 |
| WX4 | U6 promoter-gRNA-HDV | 1.14 | 0.09 |
| WUS2 | U6 promoter-gRNA-HDV | 1.31 | 0.28 |
| MS1 | U6 promoter-gRNA-HDV | 0.53 | 0.19 |
| CR82 | U6 promoter-gRNA-HDV | 1.51 | 0.23 |
| CR36 | U6 promoter-gRNA-HDV | 0.41 | 0.09 |
| WX4 | UBI-U6 promoter-C08-gRNA-C08 | 0.10 | 0.02 |
| WUS2 | UBI-U6 promoter-C08-gRNA-C08 | 0.04 | 0.04 |
| MS1 | UBI-U6 promoter-C08-gRNA-C08 | 0.02 | 0.01 |
| CR82 | UBI-U6 promoter-C08-gRNA-C08 | 0.03 | 0.01 |
| CR36 | UBI-U6 promoter-C08-gRNA-C08 | 0.05 | 0.00 |
| WX4 | UBI-U6 promoter-C08r-gRNA-C08r | 0.14 | 0.04 |
| WUS2 | UBI-U6 promoter-C08r-gRNA-C08r | 0.06 | 0.01 |
| MS1 | UBI-U6 promoter-C08r-gRNA-C08r | 0.01 | 0.00 |
| CR82 | UBI-U6 promoter-C08r-gRNA-C08r | 0.03 | 0.01 |
| CR36 | UBI-U6 promoter-C08r-gRNA-C08r | 0.01 | 0.00 |
| WX4 | UBI-U6 promoter-C13-gRNA-C13 | 0.18* | 0.01 |
| WUS2 | UBI-U6 promoter-C13-gRNA-C13 | 0.20* | 0.01 |
| MS1 | UBI-U6 promoter-C13-gRNA-C13 | 0.08* | 0.03 |
| CR82 | UBI-U6 promoter-C13-gRNA-C13 | 0.24* | 0.02 |
| CR36 | UBI-U6 promoter-C13-Grna-C13 | 0.09* | 0.01 |
| WX4 | UBI-U6 promoter-C13r-gRNA-C13r | 0.12 | 0.02 |
| WUS2 | UBI-U6 promoter-C13r-gRNA-C13r | 0.06 | 0.05 |
| MS1 | UBI-U6 promoter-C13r-gRNA-C13r | 0.00 | 0.00 |
| CR82 | UBI-U6 promoter-C13r-gRNA-C13r | 0.02 | 0.02 |
| CR36 | UBI-U6 promoter-C13r-gRNA-C13r | 0.02 | 0.00 |

To confirm the findings in Table 3, four different guide RNAs each flanked by a C13 Rnt1 recognition stem-loop were expressed together from a single Zea mays Ubiquitin promoter, 5′ UTR, and intron. As shown in Table 4, a similar increase (˜4-fold as averaged across sites) in editing activity at the CR82, CR36, WUS2, and MS1 targets was observed over the negative controls (experiments where the guide RNA or Rnt1 expression cassette was omitted). Altogether, showing the ability of Rnt1 to aid in functional guide RNA maturation from a primary transcript encoding multiple guide RNAs.

TABLE 4
Target site mutagenesis using Rnt1-mediated
guide RNA maturation (four guide RNAs)

| | Rnt1 | | Average | |
|---|---|---|---|---|
| | Expression | | Percentage of | Standard |
| Target Site | Cassette | gRNA Expression Cassette | Mutant Reads | Deviation |
| WUS2 | No | No guide RNA | 0.02 | 0.01 |
| MS1 | No | No guide RNA | 0.05 | 0.04 |
| CR82 | No | No guide RNA | 0.01 | 0.00 |
| CR36 | No | No guide RNA | 0.01 | 0.00 |
| WUS2 | No | U6 promoter-gRNA-HDV | 0.37 | 0.17 |
| MS1 | No | U6 promoter-gRNA-HDV | 0.11 | 0.08 |
| CR82 | No | U6 promoter-gRNA-HDV | 0.62 | 0.19 |
| CR36 | No | U6 promoter-gRNA-HDV | 0.19 | 0.04 |
| WUS2 | No | UBI-C13-gRNA-C13-gRNA-C13- | 0.03 | 0.01 |
| | | gRNA-C13-gRNA-C13 | | |
| MS1 | No | UBI-C13-gRNA-C13-gRNA-C13- | 0.05 | 0.02 |
| | | gRNA-C13-gRNA-C13 | | |
| CR82 | No | UBI-C13-gRNA-C13-gRNA-C13- | 0.03 | 0.01 |
| | | gRNA-C13-gRNA-C13 | | |
| CR36 | No | UBI-C13-gRNA-C13-gRNA-C13- | 0.01 | 0.00 |
| | | gRNA-C13-gRNA-C13 | | |
| WUS2 | Yes | UBI-C13-gRNA-C13-gRNA-C13- | 0.09 | 0.02 |
| | | gRNA-C13-gRNA-C13* | | |
| MS1 | Yes | UBI-C13-gRNA-C13-gRNA-C13- | 0.08 | 0.03 |
| | | gRNA-C13-gRNA-C13* | | |
| CR82 | Yes | UBI-C13-gRNA-C13-gRNA-C13- | 0.10 | 0.03 |
| | | gRNA-C13-gRNA-C13* | | |
| CR36 | Yes | UBI-C13-gRNA-C13-gRNA-C13- | 0.09 | 0.02 |
| | | gRNA-C13-gRNA-C13* | | |

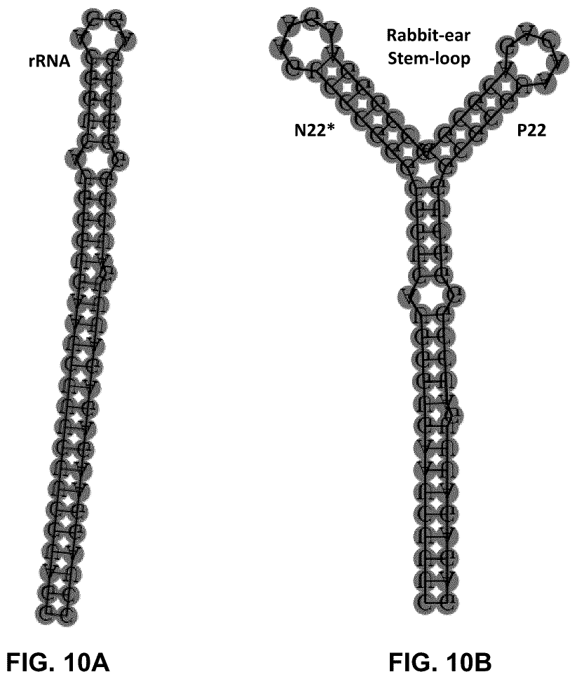

In yet another method, the RNA stem-loop structure is modified to increase binding specificity of a modified pair of yeast RNase III enzymes. For this, a sequence encoding an RNase III recognition sequence in the form of rabbit ear stem loop-forming RNA (dual RNA Stem-loop) was appended to the 5′and 3′ ends of the DNA sequence encoding each gRNA. FIGS. 10A-10B illustrate the formation of a rabbit ear stem loop from rRNA. The resulting sequence was then operably inserted into a polymerase II or III expression cassette comprising a promoter and terminator, for example but not limited to, a polymerase III U6 promoter and terminator, or a polymerase II Zea mays Ubiquitin promoter and terminator (ZM-UBI). When using the Zea mays Ubiquitin promoter, a Zea mays Ubiquitin 5′ UTR and Zea mays Ubiquitin intron 1 were also incorporated between the promoter and RNase III stem-loop recognition site. To enable expression of multiple gRNAs from a single promoter, sequences encoding two or more gRNAs flanked sequences encoding the RNase III recognition sequence were first concatenated and then linked with the respective promoter and terminator. The RNase III-gRNA-RNase III expression cassettes were then synthesized and cloned into a plasmid DNA (GenScript, USA).

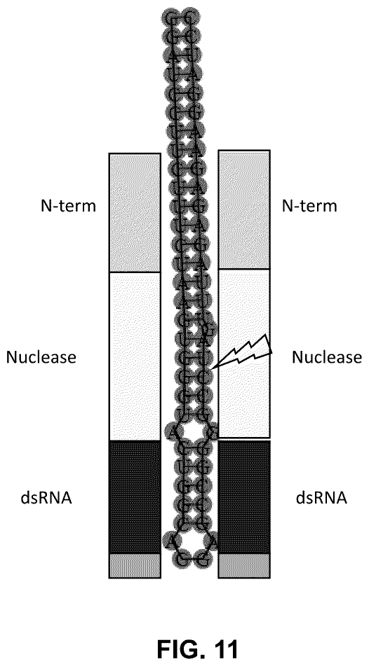

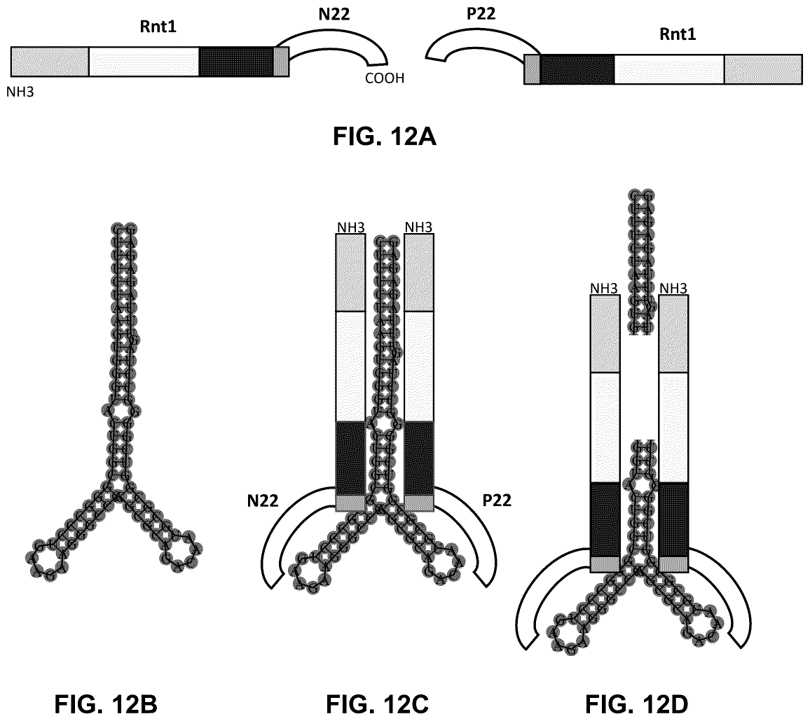

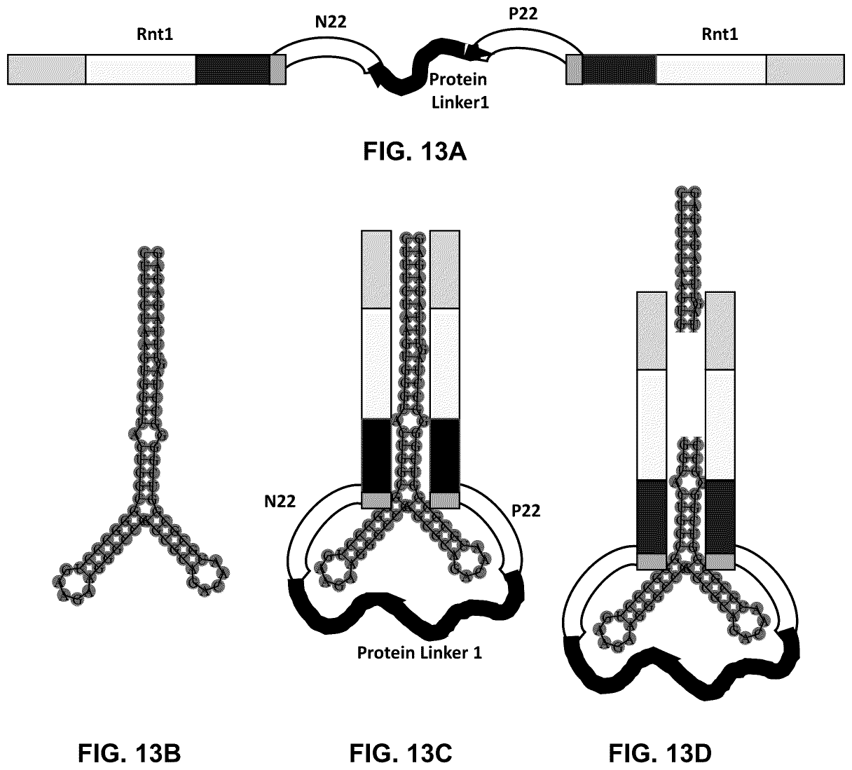

To facilitate cleavage of gRNA from a primary transcript, a maize-optimized RNase III derived from yeast Rnt1 was utilized (FIG. 12A). As compared to yeast Rnt1, which forms a homodimer (FIG. 11), the engineered RNase III is a heterodimer (FIGS. 12B-12D), wherein each subunit comprises a polypeptide domain (N22 or P22) that recognizes the stem loop of the RNase III recognition sequence. N22 is a Lambda N protein B-box and P22 is a Salmonella N protein B-box, each of which is capable of binding stem-loop structures. Upon dimerization, the RNase III heterodimer facilitates cleavage of each gRNA from the primary transcript, wherein the cleavage site depends on the distance from the stem loop. Optionally, as shown in FIGS. 13A-13D, the N22 and P22 can be operatively-associated through a linker polypeptide.

### Example 5: Mutagenesis of Multiple Genomic Target Sites

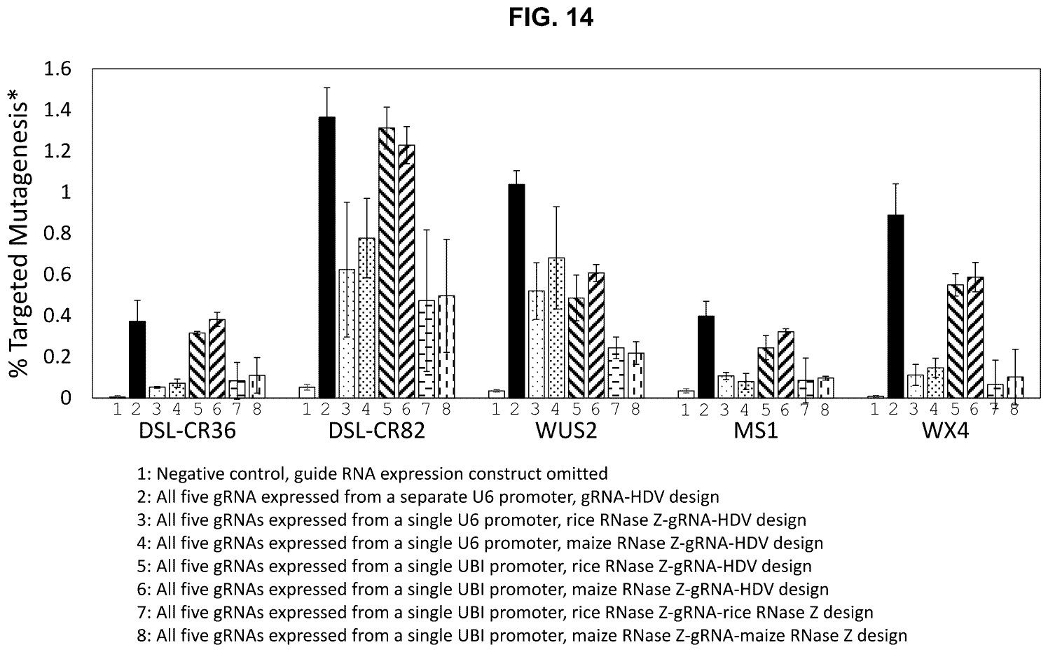

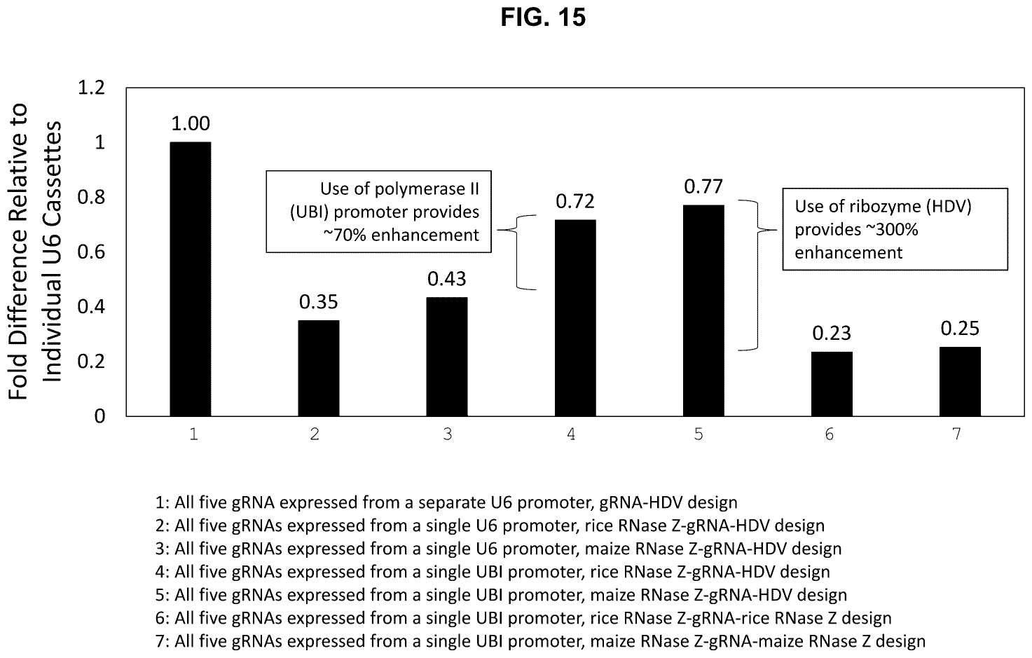

In this Example, methods of editing five genomic target sites using the RNase Z-gRNA-HDV design are described. For this, the respective gRNAs were expressed as a single transcript or polycistron from either a single Zea mays U6 or UBI promoter. As a positive control, individual U6 expression cassettes encoding each of the gRNAs in a gRNA-HDV configuration were combined and co-delivered. Experiments in which a gRNA expression construct was omitted served as a negative control. As a comparator, gRNAs expressed as a single transcript from a single Zea mays UBI promoter in an RNase Z-gRNA-RNase Z design were also tested. Across the five sites targeted (DSL-CR36, DSL-CR82, WUS2, MS1, and WX4), the polycistronic RNase Z-gRNA-HDV design demonstrated better editing than the RNase Z-gRNA-RNase Z design, relative to the positive control (FIG. 14). To quantify improvements, editing frequencies at each of the five targets were averaged for each treatment and normalized to the average editing frequency of the positive control (treatment average editing frequency/positive control average editing frequency). Using the Zea mays UBI polymerase II promoter to drive expression resulted in an approximately 70% improvement over the Zea mays U6 promoter (FIG. 15). Moreover, the inclusion of the HDV ribozyme resulted in an approximately 300% enhancement in editing efficiency relative to the RNase Z-gRNA-RNase Z design (FIG. 15). Relative to the positive control, the editing efficiency of the polycistronic RNase Z-gRNA-HDV designs (expressed from the UBI promoter) were 0.72 to 0.77, depending on the RNase Z recognition sequence used (FIG. 15). Taken together, the RNaseZ-gRNA-HDV design enables multi-locus gene editing by simplifying guide RNA delivery allowing multiple guide RNA expression cassettes to be condensed into one.

1. A method of delivering a plurality of guide polynucleotides for multiplexed genome editing of multiple genomic targets in a plant cell, the method comprising:
providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and
expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop.

2. The method of claim 1, wherein the at least one stem loop is a rabbit-ear stem loop.

3. The method of claim 1, wherein the eukaryotic RNase III is endogenous to the plant cell.

4. The method of claim 1, wherein the eukaryotic RNase III is maize-optimized yeast RNase III.

5. The method of claim 1, wherein the eukaryotic RNase III is a heterologous RNase III and the method further comprises:
engineering the heterologous RNase III to comprise a polypeptide domain capable of recognizing the stem loop of the RNA molecule.

6. The method of claim 1, wherein the eukaryotic RNase III is a heterologous RNase III and the method further comprises:
engineering the heterologous RNase III as a heterodimer molecule comprising two distinct polypeptide domains capable of recognizing two stem loops of the RNA molecule.

7. The method of claim 6, wherein the two distinct polypeptide domains are operatively-associated through a linker.

8. The method of claim 5, wherein the heterologous RNase III exhibits increased specificity to double-stranded RNA molecules such that a proportionately higher amount of the RNA molecule comprising the at least two guide polynucleotide sequences are cleaved compared to a control.

9. A composition for delivering a plurality of guide polynucleotides for multiplexed genome editing of multiple genomic targets in a plant cell, the composition comprising:
(a) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence; and
(b) a heterologous eukaryotic RNase III that cleaves an RNA molecule transcribed from the polynucleotide molecule, wherein the RNA molecule having the RNase III recognition sequence comprises at least one stem loop.

10. The composition of claim 9, wherein the heterologous eukaryotic RNase III comprises a polypeptide domain that recognizes the stem loop of the RNA molecule.

11. The composition of claim 9, wherein the heterologous eukaryotic RNase III is a heterodimer molecule comprising two distinct polypeptide domains that recognize two stem loops of the RNA molecule.

12. The composition of claim 11, wherein the two distinct polypeptide domains are operatively-associated through a linker.

13. A plant cell comprising the composition of claim 9.

14. A method for editing a plant genome, the method comprising:
providing a plant cell with:
(a) a Cas endonuclease; and
(b) a polynucleotide molecule comprising at least two guide polynucleotide sequences targeting at least two distinct genomic target sites in the plant cell, wherein the at least two guide polynucleotide sequences are separated by a double-stranded RNA-specific RNase III recognition sequence;
expressing a eukaryotic RNase III in the plant cell to cleave an RNA molecule transcribed from the polynucleotide molecule, thereby delivering the at least two guide polynucleotide sequences as a first guide polynucleotide and a second guide polynucleotide, wherein the RNA molecule having the RNase III recognition sequence comprises at least one stem loop;
introducing a first site-specific modification in a first target nucleotide sequence in the plant cell when the Cas endonuclease complexes with the first guide polynucleotide; and
introducing a second site-specific modification in a second target nucleotide sequence in the plant cell when the Cas endonuclease complexes with the second guide polynucleotide.

15. The method of claim 14, wherein the first site-specific modification in the first target nucleotide sequence is an insertion, a deletion, a substitution, a transversion, and/or a transition.

16. The method of claim 14, wherein the second site-specific modification in the second target nucleotide sequence is an insertion, a deletion, a substitution, a transversion, and/or a transition.

17. The method of claim 14, further comprising providing a donor DNA to the plant cell.

18. The method of claim 14, wherein the Cas endonuclease is a Cas12 endonuclease or a Cas9 endonuclease.

19. The method of claim 14, wherein the Cas endonuclease comprises a deactivated Cas endonuclease (dCas) complexed to a deaminase.

20. The method of claim 19, wherein the deactivated Cas endonuclease is dCas12f or dCas9.

21. The method of claim 19, wherein the deaminase is a cytosine deaminase or an adenosine deaminase.

22. The method of claim 14, wherein the Cas endonuclease comprises a deactivated Cas endonuclease (dCas) complexed to a reverse transcriptase.

23. The method of claim 22, wherein the deactivated Cas endonuclease is dCas12f or dCas9.

24. The method of claim 14, wherein the Cas endonuclease has nickase activity.

25. The method of claim 14, wherein the at least one stem loop is a rabbit-ear stem loop.

26. The method of claim 14, wherein the eukaryotic RNase III is endogenous to the plant cell.

27. The method of claim 14, wherein the eukaryotic RNase III is a heterologous RNase III and the method further comprises:
engineering the heterologous RNase III to comprise a polypeptide domain capable of recognizing the stem loop of the RNA molecule.

28. The method of claim 14, wherein the RNase III is a heterologous RNase III and the method further comprises:
engineering the heterologous RNase III as a heterodimer molecule comprising two distinct polypeptide domains capable of recognizing two stem loops of the RNA molecule.

29. The method of claim 28, wherein the two distinct polypeptide domains are operatively-associated through a linker.

30. A method of delivering a plurality of guide polynucleotides for multiplexed genome editing of multiple genomic targets in a plant cell, the method comprising:
providing a polynucleotide molecule to the plant cell, the polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end; and
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

31. The method of claim 30, wherein a ribozyme encoded by the self-cleaving ribozyme-encoding nucleotide sequence is a Hammer-head self-cleaving ribozyme.

32. A composition for delivering a plurality of guide polynucleotides for multiplexed genome editing of multiple genomic targets in a plant cell, the composition comprising a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotides is flanked by the RNase recognition sequence at a 5′ end.

33. A plant cell comprising the composition of claim 32.

34. A method for editing a plant genome, the method comprising:
providing a plant cell with:
(a) a Cas endonuclease; and
(b) a polynucleotide molecule comprising an RNase Z recognition sequence, a self-cleaving ribozyme-encoding nucleotide sequence, and at least two guide polynucleotide sequences targeting at least two distinct genomic target sites in the plant cell, wherein the at least two guide polynucleotide sequences are flanked by the self-cleaving ribozyme-encoding nucleotide sequence at a 3′ end of the polynucleotide molecule and at least one of the two guide polynucleotide sequences is flanked by the RNase Z recognition sequence at a 5′ end,
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide;
introducing a first site-specific modification in a first target nucleotide sequence in the plant cell when the Cas endonuclease complexes with the first guide polynucleotide; and
introducing a second site-specific modification in a second target nucleotide sequence in the plant cell when the Cas endonuclease complexes with the second guide polynucleotide.

35. The method of claim 34, wherein the first site-specific modification in the first target nucleotide sequence is an insertion, a deletion, a substitution, a transversion, and/or a transition.

36. The method of claim 34, wherein the second site-specific modification in the second target nucleotide sequence is an insertion, a deletion, a substitution, a transversion, and/or a transition.

37. The method of claim 34, further comprising providing a donor DNA to the plant cell.

38. The method of claim 34, wherein the Cas endonuclease is a Cas12 endonuclease or a Cas9 endonuclease.

39. The method of claim 34, wherein the Cas endonuclease comprises a deactivated Cas endonuclease (dCas) complexed to a deaminase.

40. The method of claim 39, wherein the deactivated Cas endonuclease is dCas12f or dCas9.

41. The method of claim 39, wherein the deaminase is a cytosine deaminase or an adenosine deaminase.

42. The method of claim 34, wherein the Cas endonuclease comprises a deactivated Cas endonuclease (dCas) complexed to a reverse transcriptase.

43. The method of claim 42, wherein the deactivated Cas endonuclease is dCas12f or dCas9.

44. The method of claim 34, wherein the Cas endonuclease has nickase activity.

45. The method of claim 34, wherein a ribozyme encoded by the self-cleaving ribozyme-encoding nucleotide sequence is a Hammer-head self-cleaving ribozyme.

46. A method of delivering a plurality of guide polynucleotides for multiplexed genome editing of multiple genomic targets in a plant cell, the method comprising:
providing a polynucleotide molecule to the plant cell, the polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and
expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop.

47. The method of claim 46, wherein the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

48. The method of claim 47, wherein the ribonuclease domain of the viral coat protein comprises a PilT N-terminus (PIN) ribonuclease domain.

49. The method of claim 48, wherein the PIN ribonuclease domain is from a human telomerase-binding protein EST1A.

50. The method of claim 47, wherein the viral coat protein is a MS2 bacteriophage coat protein.

51. The method of claim 46, wherein the 5′ and 3′ ends of the polynucleotide molecule comprising at least two guide polynucleotide sequences are flanked with polynucleotide sequences encoding stem loop-forming RNA.

52. The method of claim 50, further comprising engineering the MS2 bacteriophage coat protein to comprise a polypeptide domain capable of recognizing the stem loop of the RNA molecule.

53. The method of claim 50, wherein the MS2 bacteriophage coat protein is a homodimer molecule comprising two polypeptide domains capable of recognizing two stem loops of the RNA molecule.

54. The method of claim 47, wherein the viral coat protein is a PP7 bacteriophage coat protein.

55. The method of claim 54, wherein the 5′ end of the polynucleotide molecule comprising at least two guide polynucleotide sequences is flanked with a polynucleotide sequence encoding stem loop-forming RNA and the 3′ end of the polynucleotide molecule comprising at least two guide polynucleotide sequences is flanked with a polynucleotide sequence encoding a ribozyme.

56. The method of claim 54, further comprising engineering the PP7 bacteriophage coat protein to comprise a polypeptide domain capable of recognizing the stem loop of the RNA molecule.

57. A composition for delivering a plurality of guide polynucleotides for multiplexed genome editing of multiple genomic targets in a plant cell, the composition comprising:
(c) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence; and
(d) a double-stranded RNA-specific protein comprising a ribonuclease domain that cleaves an RNA molecule transcribed from the polynucleotide molecule, wherein the RNA molecule having the double-stranded RNA-specific protein recognition sequence comprises at least one stem loop.

58. The composition of claim 57, wherein the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

59. The composition of claim 58, wherein the ribonuclease domain of the viral coat protein comprises a PilT N-terminus (PIN) ribonuclease domain.

60. The composition of claim 59, wherein the PIN ribonuclease domain is from a human telomerase-binding protein EST1A.

61. The composition of claim 57, wherein the viral coat protein is a MS2 bacteriophage coat protein.

62. The composition of claim 61, wherein the MS2 bacteriophage coat protein comprises a polypeptide domain capable of recognizing the stem loop of the RNA molecule.

63. The composition of claim 61, wherein the MS2 bacteriophage coat protein is a homodimer molecule comprising two polypeptide domains capable of recognizing two stem loops of the RNA molecule.

64. The composition of claim 57, wherein the viral coat protein is a PP7 bacteriophage coat protein.

65. The composition of claim 64, wherein the PP7 bacteriophage coat protein comprises a polypeptide domain capable of recognizing the stem loop of the RNA molecule.

66. A plant cell comprising the composition of claim 57.

67. A method for editing a plant genome, the method comprising:
providing a plant cell with:
(c) a Cas endonuclease; and
(d) a polynucleotide molecule comprising at least two guide polynucleotide sequences targeting at least two distinct genomic target sites in the plant cell, wherein the at least two guide polynucleotide sequences are separated by a double-stranded RNA-specific protein recognition sequence;
expressing a double-stranded RNA-specific protein comprising a ribonuclease domain in the plant cell to cleave an RNA molecule transcribed from the polynucleotide molecule, thereby delivering the at least two guide polynucleotide sequences as a first guide polynucleotide and a second guide polynucleotide, wherein the RNA molecule having the double-stranded RNA-specific protein recognition sequence comprises at least one stem loop;
introducing a first site-specific modification in a first target nucleotide sequence in the plant cell when the Cas endonuclease complexes with the first guide polynucleotide; and
introducing a second site-specific modification in a second target nucleotide sequence in the plant cell when the Cas endonuclease complexes with the second guide polynucleotide.

68. The method of claim 67, wherein the double-stranded RNA-specific protein is a viral coat protein comprising the ribonuclease domain, the double-stranded RNA-specific protein recognition sequence of the polynucleotide molecule is a viral coat protein recognition sequence, and the double-stranded RNA-specific protein recognition sequence of the RNA molecule is a viral coat protein comprising the at least one stem loop.

69. The method of claim 67, wherein the first site-specific modification in the first target nucleotide sequence is an insertion, a deletion, a substitution, a transversion, and/or a transition.

70. The method of claim 67, wherein the second site-specific modification in the second target nucleotide sequence is an insertion, a deletion, a substitution, a transversion, and/or a transition.

71. The method of claim 67, further comprising providing a donor DNA to the plant cell.

72. The method of claim 67, wherein the Cas endonuclease is a Cas12 endonuclease or a Cas9 endonuclease.

73. The method of claim 67, wherein the Cas endonuclease comprises a deactivated Cas endonuclease (dCas) complexed to a deaminase.

74. The method of claim 73, wherein the deactivated Cas endonuclease is dCas12f or dCas9.

75. The method of claim 73, wherein the deaminase is a cytosine deaminase or an adenosine deaminase.

76. The method of claim 67, wherein the Cas endonuclease comprises a deactivated Cas endonuclease (dCas) complexed to a reverse transcriptase.

77. The method of claim 76, wherein the deactivated Cas endonuclease is dCas12f or dCas9.

78. The method of claim 67, wherein the Cas endonuclease has nickase activity.

79. The method of claim 68, wherein the ribonuclease domain of the viral coat protein comprises a PilT N-terminus (PIN) ribonuclease domain.

80. The method of claim 79, wherein the PIN ribonuclease domain is from a human telomerase-binding protein EST1A.

81. The method of claim 68, wherein the viral coat protein is a MS2 bacteriophage coat protein.

82. The method of claim 81, further comprising engineering the MS2 bacteriophage coat protein to comprise a polypeptide domain capable of recognizing the stem loop of the RNA molecule.

83. The method of claim 81, wherein the MS2 bacteriophage coat protein is a homodimer molecule comprising two polypeptide domains capable of recognizing two stem loops of the RNA molecule.

84. The method of claim 68, wherein the viral coat protein is a PP7 bacteriophage coat protein.

85. The method of claim 84, further comprising engineering the PP7 bacteriophage coat protein to comprise a polypeptide domain capable of recognizing the stem loop of the RNA molecule.

86. The method of claim 67, wherein the 5′ and 3′ ends of the polynucleotide molecule comprising the at least two guide polynucleotide sequences are flanked with polynucleotide sequences encoding stem loop-forming RNA.

87. The method of claim 67, wherein the 5′ end of the polynucleotide molecule comprising the at least two guide polynucleotide sequences is flanked with a polynucleotide sequence encoding stem loop-forming RNA and the 3′ end of the polynucleotide molecule comprising the at least two guide polynucleotide sequences is flanked with a polynucleotide sequence encoding a ribozyme.

88. The method of claim 14, wherein the eukaryotic RNase III is maize-optimized yeast RNase III.

89. A method of delivering a plurality of guide polynucleotides for multiplexed genome editing of multiple genomic targets in a plant cell, the method comprising:
providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by an RNase Z recognition sequence; and
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

90. A composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by an RNase recognition sequence.

91. A plant cell comprising the composition of claim 90.

92. A method for editing a plant genome, the method comprising:
providing a plant cell with:
(c) a Cas endonuclease; and
(d) a polynucleotide molecule comprising at least two guide polynucleotide sequences targeting at least two distinct genomic target sites in the plant cell, wherein each of the at least two guide polynucleotide sequences is flanked by an RNase Z recognition sequence,
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide;
introducing a first site-specific modification in a first target nucleotide sequence in the plant cell when the Cas endonuclease complexes with the first guide polynucleotide; and
introducing a second site-specific modification in a second target nucleotide sequence in the plant cell when the Cas endonuclease complexes with the second guide polynucleotide.

93. The method of claim 92, wherein the first site-specific modification in the first target nucleotide sequence is an insertion, a deletion, a substitution, a transversion, and/or a transition.

94. The method of claim 92, wherein the second site-specific modification in the second target nucleotide sequence is an insertion, a deletion, a substitution, a transversion, and/or a transition.

95. The method of claim 92, further comprising providing a donor DNA to the plant cell.

96. The method of claim 92, wherein the Cas endonuclease is a Cas12 endonuclease or a Cas9 endonuclease.

97. The method of claim 92, wherein the Cas endonuclease comprises a deactivated Cas endonuclease (dCas) complexed to a deaminase.

98. The method of claim 97, wherein the deactivated Cas endonuclease is dCas12f or dCas9.

99. The method of claim 97, wherein the deaminase is a cytosine deaminase or an adenosine deaminase.

100. The method of claim 92, wherein the Cas endonuclease comprises a deactivated Cas endonuclease (dCas) complexed to a reverse transcriptase.

101. The method of claim 100, wherein the deactivated Cas endonuclease is dCas12f or dCas9.

102. The method of claim 92, wherein the Cas endonuclease has nickase activity.

103. A method of delivering a plurality of guide polynucleotides for multiplexed genome editing of multiple genomic targets in a plant cell, the method comprising:
providing a polynucleotide molecule to the plant cell, the polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence; and
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide.

104. The method of claim 103, wherein a ribozyme encoded by each of the self-cleaving ribozyme-encoding nucleotide sequences is a Hammer-head self-cleaving ribozyme.

105. A composition for delivering a plurality of guide polynucleotides for multiplexed genome editing of multiple genomic targets in a plant cell, the composition comprising a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence.

106. A plant cell comprising the composition of claim 105.

107. A method for editing a plant genome, the method comprising:
providing a plant cell with:
(e) a Cas endonuclease; and
(f) a polynucleotide molecule comprising at least two guide polynucleotide sequences targeting at least two distinct genomic target sites in the plant cell, wherein each of the at least two guide polynucleotide sequences is flanked by a self-cleaving ribozyme-encoding nucleotide sequence,
expressing the polynucleotide molecule in the plant cell to cleave an RNA molecule transcribed from the polynucleotide molecule, thereby delivering the at least two guide polynucleotide sequences as a first guide polynucleotide and a second guide polynucleotide;
introducing a first site-specific modification in a first target nucleotide sequence in the plant cell when the Cas endonuclease complexes with the first guide polynucleotide; and
introducing a second site-specific modification in a second target nucleotide sequence in the plant cell when the Cas endonuclease complexes with the second guide polynucleotide.

108. The method of claim 107, wherein the first site-specific modification in the first target nucleotide sequence is an insertion, a deletion, a substitution, a transversion, and/or a transition.

109. The method of claim 107, wherein the second site-specific modification in the second target nucleotide sequence is an insertion, a deletion, a substitution, a transversion, and/or a transition.

110. The method of claim 107, further comprising providing a donor DNA to the plant cell.

111. The method of claim 107, wherein the Cas endonuclease is a Cas12 endonuclease or a Cas9 endonuclease.

112. The method of claim 110, wherein the Cas endonuclease comprises a deactivated Cas endonuclease (dCas) complexed to a deaminase.

113. The method of claim 112, wherein the deactivated Cas endonuclease is dCas12f or dCas9.

114. The method of claim 112, wherein the deaminase is a cytosine deaminase or an adenosine deaminase.

115. The method of claim 107, wherein the Cas endonuclease comprises a deactivated Cas endonuclease (dCas) complexed to a reverse transcriptase.

116. The method of claim 115, wherein the deactivated Cas endonuclease is dCas12f or dCas9.

117. The method of claim 107, wherein the Cas endonuclease has nickase activity.

118. The method of claim 107, wherein a ribozyme encoded by the self-cleaving ribozyme-encoding nucleotide sequence is a Hammer-head self-cleaving ribozyme.

119. A method for generating a plurality of guide RNA molecules for genetic modification in a plant cell, the method comprising providing to the plant cell a polynucleotide expression cassette encoding two or more guide RNA sequences separated by one or more tRNA cleavage sequence, wherein each guide RNA sequence comprises a 3′ spacer sequence that exhibits complementarity to a target sequence in the plant cell, wherein the plant cell's tRNA processing system cleaves a transcript generated from the transcribed polynucleotide expression cassette, thereby generating the plurality of guide RNA sequences.

120. The method of claim 119, wherein the guide RNA sequences target multiple sites in one or more chromosome of the plant cell.

121. The method of claim 119, wherein said guide RNA sequences target one or more multigene families in the plant cell.

122. The method of claim 119, wherein the genetic modification is RNA guided chromosomal genome modification in the presence of a CRISPR-associated polypeptide.

123. The method of claim 119, wherein the genetic modification is a chromosomal genome modification selected from the group consisting of targeted mutation, homology-dependent repair, homology directed recombination, transcriptional activation, transcriptional downregulation, insertion, deletion, epigenome modification, and a combination of the foregoing.

124. The method of claim 119, wherein the genetic modification is RNA guided base editing.

125. The method of claim 119, wherein the tRNA cleavage sequence includes a pretRNA acceptor stem, a D-loop arm and a TΨC-loop arm.

126. The method of claim 119, wherein the tRNA cleavage sequence includes an active site for one or more of RNase P and/or RNase Z and/or RNase E.

127. A nucleic acid expression cassette for generating a plurality of guide RNA molecules for genetic modification in a plant cell comprising a polynucleotide encoding two or more guide RNA sequences separated by one or more tRNA cleavage sequence, wherein each guide RNA sequence comprises a 3′ spacer sequence that exhibits complementarity to a target sequence in the plant cell.

128. The nucleic acid expression cassette of claim 127, wherein the tRNA cleavage sequence includes a pretRNA acceptor stem, a D-loop arm and a TΨC-loop arm.

129. The nucleic acid expression cassette of claim 127, wherein the tRNA cleavage sequence includes an active site for one or more of RNase P and/or RNase Z and/or RNase E.

130. The nucleic acid expression cassette of claim 127, wherein the nucleic acid expression cassette includes a guide RNA-tRNA-guide RNA configuration such that the spacer sequence is at the 3′ position.


SEQUENCE LISTING (74 sequences)

SEQ ID NO: 1 - protein, 1368 aa, Steptococcus pyogenes [sequence omitted]

SEQ ID NO: 2 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 3 - protein, 497 aa, Synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 4 - protein, 497 aa, Synthetic construct
MGESVKAIKLKILDMFLDPECTKQDDNWRKDLSTMSRFCGEAGNSCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 5 - protein, 422 aa, Acidibacillus sulfuroxidans
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 6 - protein, 422 aa, Synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDYPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLTQGKYAGGARGGHGRDKRIKPIEQLRDKIPNFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 7 - protein, 18 aa, Agrobacterium tumefaciens
KRPRDRHDGELGGRKRAR

SEQ ID NO: 8 - DNA, 896 bp, Zea mays
  source 1..896: mol_type = unassigned DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 9 - DNA, 82 bp, Zea mays
  source 1..82: mol_type = unassigned DNA
tccccaacctcgtgttgttcggagcgcacacacacacaaccagatctcccccaaatccacccgtcggcacctccgcttcaag

SEQ ID NO: 10 - DNA, 1013 bp, Zea mays
  source 1..1013: mol_type = unassigned DNA
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 11 - DNA, 1680 bp, Synthetic construct
  source 1..1680: mol_type = other DNA
atgggcgagagcgtcaaggcaataaaattaaagatcctcgacatgttcctcgaccccgagtgcaccaagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggacgacaactggcgcaaggacctcagcaccatgagccgcttctgcggcgaggccggcaacatgtgcctcagggacctctacaactacttcagcatgcccaaggaggaccgcatcagctccaaggacttatataacgccatgtaccataaaactaagctcctccaccccggcctccccgggaaggtgaagaaccaaatcgtcaaccacgccaaggacgtctggaagcgcaacgccaagctcatctaccgcaaccaaatcagcatgcccacatataagatcaccaccgcccccatccgcctccaaaataacatctacaaattaataaaaaataagaacaaatacataatcgacgtccagctctacagcaaggagtacagcaaggacagcggcaagggcacccacaggtacttcctcgtcgccgtcagggacagcagcaccaggatgatcttcgacaggatcatgagcaaggaccacatcgacagcagcaagagctacaagcagggccagctccaaatcaagaaggaccaccagggcaagtggtactgcatcatcccctatacattccccacccacgaacacgtcctcgaccccgacaaggtcatgggcgtcgacctcggggtggctaaggccgtctactgggctttcaacagcagctataaaagaggctgcatcgacggcggcgagatcgagcacttcaggaagatgatcagggcccggcgcgtcagcatccagaatcaaatcaaacacagcggcgacgcccgcaagggccacggcaggaagagggccctcaagcccatcgaaaccctcagcgagaaggagagcaacttccgcgacacaataaacttcaggtacgccaacaggatcgtcgaggccgctatcaagcagggctgcggcaccatccagatcgagaacctcgagggcatcgctgacaggaccggcagcaagttcctcaagaactggccctactacgacctccagaccaagatcgtcaataaagccaaggagcacggcatcaccgtcgtcgcaataaacccccagtatacatcccagcgctgcagcatgtgcggctacatcgagaaaaccaacaggagcagccaggccgtgttcgagtgcaagcagtgcggctacggcagccgcaccaactgcatcaactgcaggcacgtccaagtctccggcgacgtctgcgaggagtgcggcggcatcgtcaagaaggagaacgtcaacgccgactacaacgccgccaagaacatcagcaccccctacatcgaccagataataatggagaagtgcctcgagctcggcatcccctaccgctccatcacctgcaaggagtgcggccacatccaggctagcggcaacacctgcgaggtctgcggcagcaccaacatcctcaaaccaaagaagatccgcaaggcaaaa

SEQ ID NO: 12 - DNA, 1491 bp, Synthetic construct
  source 1..1491: mol_type = other DNA
atgggcgagagcgtcaaggcaataaaattaaagatcctcgacatgttcctcgaccccgagtgcaccaagcaggacgacaactggcgcaaggacctcagcaccatgagccgcttctgcggcgaggccggcaacatgtgcctcagggacctctacaactacttcagcatgcccaaggaggaccgcatcagctccaaggacttatataacgccatgtaccataaaactaagctcctccaccccggcctccccgggaaggtgaagaaccaaatcgtcaaccacgccaaggacgtctggaagcgcaacgccaagctcatctaccgcaaccaaatcagcatgcccacatataagatcaccaccgcccccatccgcctccaaaataacatctacaaattaataaaaaataagaacaaatacataatcgacgtccagctctacagcaaggagtacagcaaggacagcggcaagggcacccacaggtacttcctcgtcgccgtcagggacagcagcaccaggatgatcttcgacaggatcatgagcaaggaccacatcgacagcagcaagagctacaagcagggccagctccaaatcaagaaggaccaccagggcaagtggtactgcatcatcccctatacattccccacccacgaacacgtcctcgaccccgacaaggtcatgggcgtcgacctcggggtggctaaggccgtctactgggctttcaacagcagctataaaagaggctgcatcgacggcggcgagatcgagcacttcaggaagatgatcagggcccggcgcgtcagcatccagaatcaaatcaaacacagcggcgacgcccgcaagggccacggcaggaagagggccctcaagcccatcgaaaccctcagcgagaaggagagcaacttccgcgacacaataaacttcaggtacgccaacaggatcgtcgaggccgctatcaagcagggctgcggcaccatccagatcgagaacctcgagggcatcgctgacaggaccggcagcaagttcctcaagaactggccctactacgacctccagaccaagatcgtcaataaagccaaggagcacggcatcaccgtcgtcgcaataaacccccagtatacatcccagcgctgcagcatgtgcggctacatcgagaaaaccaacaggagcagccaggccgtgttcgagtgcaagcagtgcggctacggcagccgcaccaactgcatcaactgcaggcacgtccaagtctccggcgacgtctgcgaggagtgcggcggcatcgtcaagaaggagaacgtcaacgccgactacaacgccgccaagaacatcagcaccccctacatcgaccagataataatggagaagtgcctcgagctcggcatcccctaccgctccatcacctgcaaggagtgcggccacatccaggctagcggcaacacctgcgaggtctgcggcagcaccaacatcctcaaaccaaagaagatccgcaaggcaaaa

SEQ ID NO: 13 - DNA, 189 bp, Solanum tuberosum
  source 1..189: mol_type = unassigned DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 14 - DNA, 21 bp, Synthetic construct
  source 1..21: mol_type = other DNA
ccgaagaagaagcgcaaggtg

SEQ ID NO: 15 - DNA, 910 bp, Zea mays
  source 1..910: mol_type = unassigned DNA
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgac

SEQ ID NO: 16 - DNA, 1001 bp, Zea mays
  source 1..1001: mol_type = unassigned DNA
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattg

SEQ ID NO: 17 - DNA, 77 bp, Oryza sativa
  source 1..77: mol_type = unassigned DNA
aacaaagcaccagtggtctagtggtagaatagtaccctgccacggtacagacccgggttcgattcccggctggtgca

SEQ ID NO: 18 - DNA, 77 bp, Zea mays
  source 1..77: mol_type = unassigned DNA
acaaaagcaccagtggtctagtggtagaatagtaccctgccacggtacagacccgggttcgattcccggctggtgca

SEQ ID NO: 19 - DNA, 189 bp, Zea mays
  source 1..189: mol_type = unassigned DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 20 - DNA, 20 bp, Zea mays
  source 1..20: mol_type = unassigned DNA
aatgccgcagttcattgagc

SEQ ID NO: 21 - DNA, 20 bp, Zea mays
  source 1..20: mol_type = unassigned DNA
tcatgtctactggtctattt

SEQ ID NO: 22 - DNA, 20 bp, Zea mays
  source 1..20: mol_type = unassigned DNA
agttcagagaaggcaacctt

SEQ ID NO: 23 - DNA, 20 bp, Zea mays
  source 1..20: mol_type = unassigned DNA
cacagggagggagcaaacaa

SEQ ID NO: 24 - DNA, 20 bp, Zea mays
  source 1..20: mol_type = unassigned DNA
aagttcacggcgttccaggc

SEQ ID NO: 25 - DNA, 68 bp, Synthetic construct
  source 1..68: mol_type = other DNA
ggccggcatggtcccagcctcctcgctggcgccggctgggcaacatgcttcggcatggcgaatgggac

SEQ ID NO: 26 - skipped

SEQ ID NO: 27 - protein, 181 aa, Homo sapiens
MELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVG

SEQ ID NO: 28 - protein, 130 aa, Synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 29 - protein, 128 aa, Pseudomonas phage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 30 - protein, 30 aa, Synthetic construct
SAGGGGSGGGGSGGGGSGPKKKRKVAAAGS

SEQ ID NO: 31 - protein, 21 aa, Synthetic construct
KRPRDRHDGELGGRKRARGRA

SEQ ID NO: 32 - protein, 27 aa, Synthetic construct
KRPRDRHDGELGGRKRARGGSGGSGGS

SEQ ID NO: 33 - protein, 28 aa, Synthetic construct
KRPRDRHDGELGGRKRARGRAVDTGNGS

SEQ ID NO: 34 - protein, 14 aa, Synthetic construct
SRADPKKKRKVGRA

SEQ ID NO: 35 - protein, 20 aa, Synthetic construct
SRADPKKKRKVGGSGGSGGS

SEQ ID NO: 36 - protein, 17 aa, Synthetic construct
SRADPKKKRKVDTGNGS

SEQ ID NO: 37 - DNA, 19 bp, Escherichia phage MS2
  source 1..19: mol_type = unassigned DNA
acatgaggatcacccatgt

SEQ ID NO: 38 - protein, 25 aa, Pseudomonas phage PP7
TAAGGAGTTTATATGGAAACCCTTA

SEQ ID NO: 39 - skipped

SEQ ID NO: 40 - DNA, 971 bp, Zea mays
  source 1..971: mol_type = unassigned DNA
ccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctag

SEQ ID NO: 41 - DNA, 91 bp, Zea mays
  source 1..91: mol_type = unassigned DNA
ctctagcaaagcacttgccatctaccgaccgccgcattccaaacagcccgacgagctagcagagcggcaggcacctccctcctcaaggaac

SEQ ID NO: 42 - DNA, 43 bp, Synthetic construct
  source 1..43: mol_type = other DNA
gtaaatctgatgagtccgtgaggacgaaacgagtaagctcgtc

SEQ ID NO: 43 - DNA, 12 bp, Synthetic construct
  source 1..12: mol_type = other DNA
aggattattccg

SEQ ID NO: 44 - DNA, 1416 bp, Saccharomyces cerevisiae
  source 1..1416: mol_type = unassigned DNA
atgggctcaaaagtagcaggtaaaaagaaaacccagaatgataataaactagataacgaaaatggttcacagcagcgcgaaaatatcaataccaagacgcttttgaaaggcaaccttaagatatcaaattacaaatatcttgaggtgattcaactagaacatgctgtgacaaaattggtggagtcttacaataaaataattgaactttcaccaaatttggtagcttacaatgaagccgttaacaatcaagatagagtgcctgttcagatacttccttctctatcacgttatcaattaaaactggcagctgaattaaaaaccttacatgatcttaaaaaagacgcaattttgacagaaattactgattatgaaaatgaatttgatactgaacaaaagcaacctatattgcaagaaatcagtaaagctgatatggaaaagttagagaagctagaacaagttaaaagggaaaagcgagagaaaatagatgtaaatgtgtacgaaaatttaaatgaaaaggaagatgaagaagaagacgaaggagaagatagctatgacccaacaaaggctggtgatatcgtcaaggcaacaaaatggcctccaaaattaccagagattcaagatttagcgattagggccagggtatttattcacaaatccacaataaaggataaagtttacttgtctggatcagaaatgatcaacgcacataatgaaagactagaattcctaggcgattcgatcttaaattctgttatgacattaattatttataacaagtttcccgattacagcgagggtcagttatcaacattaaggatgaatttggtaagcaacgaacagatcaaacaatggtcaataatgtacaatttccatgagaaacttaagacaaattttgacttgaaggatgaaaattccaattttcaaaacggcaaactaaaactgtatgcagatgtgttcgaagcttacattggcggcttgatggaagatgatccaaggaataatttgcccaaaataagaaaatggttaagaaagcttgccaaacctgtcattgaggaggctacccgtaatcaagttgccttggagaagacggataaacttgatatgaatgctaaaaggcagctttactctttgattggctatgcttcattacgtctacattatgttaccgtaaaaaaacccactgcagttgatcctaattccatagttgagtgcagagtaggtgatggaacagttttagggaccggtgtgggcagaaatatcaaaattgcgggtattagggctgcagaaaatgctcttcgtgacaaaaaaatgttagatttttacgccaaacaaagagctgccattcctaggagtgaatctgtgttaaaagatccctcacaaaagaataagaaaagaaaattctcagatacaagctga

SEQ ID NO: 45 - protein, 471 aa, Saccharomyces cerevisiae
MGSKVAGKKKTQNDNKLDNENGSQQRENINTKTLLKGNLKISNYKYLEVIQLEHAVTKLVESYNKIIELSPNLVAYNEAVNNQDRVPVQILPSLSRYQLKLAAELKTLHDLKKDAILTEITDYENEFDTEQKQPILQEISKADMEKLEKLEQVKREKREKIDVNVYENLNEKEDEEEDEGEDSYDPTKAGDIVKATKWPPKLPEIQDLAIRARVFIHKSTIKDKVYLSGSEMINAHNERLEFLGDSILNSVMTLIIYNKFPDYSEGQLSTLRMNLVSNEQIKQWSIMYNFHEKLKTNFDLKDENSNFQNGKLKLYADVFEAYIGGLMEDDPRNNLPKIRKWLRKLAKPVIEEATRNQVALEKTDKLDMNAKRQLYSLIGYASLRLHYVTVKKPTAVDPNSIVECRVGDGTVLGTGVGRNIKIAGIRAAENALRDKKMLDFYAKQRAAIPRSESVLKDPSQKNKKRKFSDTS

SEQ ID NO: 46 - DNA, 1416 bp, Synthetic construct
  source 1..1416: mol_type = other DNA
atgggcagcaaggtggccggcaagaagaagacccagaacgacaacaagctcgacaacgagaacggcagccagcagagggagaacatcaacaccaagaccctcctcaagggcaacctcaagatcagcaactacaagtacctcgaggtgatccagctcgagcacgccgtgaccaagctcgtggagagctacaacaagatcatcgagctcagcccgaacctcgtggcctacaacgaggccgtgaacaaccaggacagggtgccggtgcagatcctcccgagcctcagcaggtaccagctcaagctcgccgccgagctcaagaccctccacgacctcaagaaggacgccatcctcaccgagatcaccgactacgagaacgagttcgacaccgagcagaagcagccgatcctccaggagatcagcaaggccgacatggagaagctcgagaagctcgagcaggtgaagagggagaagagggagaagatcgacgtgaacgtgtacgagaacctcaacgagaaggaggacgaggaggaggacgagggcgaggacagctacgacccgaccaaggccggcgacatcgtgaaggccaccaagtggccgccgaagctcccggagatccaggacctcgccatcagggccagggtgttcatccacaagagcaccatcaaggacaaggtgtacctcagcggcagcgagatgatcaacgcccacaacgagaggctcgagttcctcggcgacagcatcctcaacagcgtgatgaccctcatcatctacaacaagttcccggactacagcgagggccagctcagcaccctcaggatgaacctcgtgagcaacgagcagatcaagcagtggagcatcatgtacaacttccacgagaagctcaagaccaacttcgacctcaaggacgagaacagcaacttccagaacggcaagctcaagctctacgccgacgtgttcgaggcctacatcggcggcctcatggaggacgacccgaggaacaacctcccgaagatcaggaagtggctcaggaagctcgccaagccggtgatcgaggaggccaccaggaaccaggtggccctcgagaagaccgacaagctcgacatgaacgccaagaggcagctctacagcctcatcggctacgccagcctcaggctccactacgtgaccgtgaagaagccgaccgccgtggacccgaacagcatcgtggagtgcagggtgggcgacggcaccgtgctcggcaccggcgtgggcaggaacatcaagatcgccggcatcagggccgccgagaacgccctcagggacaagaagatgctcgacttctacgccaagcagagggccgccatcccgaggagcgagagcgtgctcaaggacccgagccagaagaacaagaagaggaagttcagcgacaccagctag

SEQ ID NO: 47 - RNA, 48 nt, Synthetic construct
  source 1..48: mol_type = other RNA
ggcgtttagatgtcatgtcatgagtccatggcatggcagttagacgcc

SEQ ID NO: 48 - RNA, 48 nt, Synthetic construct
  source 1..48: mol_type = other RNA
ggcgctatcgtgtcatgtcatgagtccatggcatggcatgatagcgcc

SEQ ID NO: 49 - RNA, 56 nt, Saccharomyces cerevisiae
  source 1..56: mol_type = unassigned RNA
tttcctttgacttcgcatcatgaatgagttcattcatgatgaagcgaaaggagaga

SEQ ID NO: 50 - RNA, 50 nt, Saccharomyces cerevisiae
  source 1..50: mol_type = unassigned RNA
cttttctacaaatttgattcatgagtccatggaacaaatatatagaactc

SEQ ID NO: 51 - RNA, 34 nt, Saccharomyces cerevisiae
  source 1..34: mol_type = unassigned RNA
actgggatatatgtgagttcacgtgcgtcccaat

SEQ ID NO: 52 - RNA, 51 nt, Saccharomyces cerevisiae
  source 1..51: mol_type = unassigned RNA
tttctttctaagtgggtactggcaggagtcggggcctagtttagagagaag

SEQ ID NO: 53 - DNA, 48 bp, Synthetic construct
  source 1..48: mol_type = other DNA
ggcgtttagatgtcatgtcatgagtccatggcatggcagttagacgcc

SEQ ID NO: 54 - DNA, 48 bp, Synthetic construct
  source 1..48: mol_type = other DNA
ggcgctatcgtgtcatgtcatgagtccatggcatggcatgatagcgcc

SEQ ID NO: 55 - DNA, 56 bp, Synthetic construct
  source 1..56: mol_type = other DNA
tttcctttgacttcgcatcatgaatgagttcattcatgatgaagcgaaaggagaga

SEQ ID NO: 56 - DNA, 50 bp, Synthetic construct
  source 1..50: mol_type = other DNA
cttttctacaaatttgattcatgagtccatggaacaaatatatagaactc

SEQ ID NO: 57 - DNA, 34 bp, Synthetic construct
  source 1..34: mol_type = other DNA
actgggatatatgtgagttcacgtgcgtcccaat

SEQ ID NO: 58 - DNA, 51 bp, Synthetic construct
  source 1..51: mol_type = other DNA
tttctttctaagtgggtactggcaggagtcggggcctagtttagagagaag

SEQ ID NO: 59 - DNA, 402 bp, Lambda bacteriophage
  source 1..402: mol_type = unassigned DNA
atgtgccaatcgcggggggttttcgttcaggactacaactgccacacaccaccaaagctaactgacaggagaatccagatggatgcacaaacacgccgccgcgaacgtcgcgcagagaaacaggctcaatggaaagcagcaaatcccctgttggttggggtaagcgcaaaaccagttaaccgccctattctctcgctgaatcgcaaaccgaaatcacgagtagaaagcgcactaaatccgatagaccttacagtgctggctgaataccacaaacagattgaaagcaacctgcaacgtattgagcgcaagaatcagcgcacatggtacagcaagcctggcgaacgcggcataacatgcagtggacgccagaaaattaagggaaaatcgattcctcttatctag

SEQ ID NO: 60 - protein, 133 aa, Lambda bacteriophage
MCQSRGVFVQDYNCHTPPKLTDRRIQMDAQTRRRERRAEKQAQWKAANPLLVGVSAKPVNRPILSLNRKPKSRVESALNPIDLTVLAEYHKQIESNLQRIERKNQRTWYSKPGERGITCSGRQKIKGKSIPLI

SEQ ID NO: 61 - DNA, 66 bp, Synthetic construct
  source 1..66: mol_type = other DNA
atggatgcacaaacacgccgccgcgaacgtcgcgcagagaaacaggctcaatggaaagcagcaaat

SEQ ID NO: 62 - protein, 22 aa, Synthetic construct
MDAQTRRRERRAEKQAQWKAAN

SEQ ID NO: 63 - DNA, 303 bp, Salmonella phage
  source 1..303: mol_type = unassigned DNA
atgacggttatcacctacgggaagtcaacgtttgcaggcaatgctaaaactcgccgtcatgagcggcgcagaaagctagccatagagcgcgacaccatctgcaatatcatcgattcaatttttggctgcgatgctcctgatgcttctcaggaagttaaagccaaaagaattgaccgtgtcaccaaagccatttcgcttgccggaacgcgtcagaaggaagttgaaggaggatctgtacttcttccaggcgtagcactttacgcggctggtcatcgtaagagcaaacaaataacagcgaggtaa

SEQ ID NO: 64 - protein, 100 aa, Salmonella phage
MTVITYGKSTFAGNAKTRRHERRRKLAIERDTICNIIDSIFGCDAPDASQEVKAKRIDRVTKAISLAGTRQKEVEGGSVLLPGVALYAAGHRKSKQITAR

SEQ ID NO: 65 - DNA, 63 bp, Synthetic construct
  source 1..63: mol_type = other DNA
ggcaatgctaaaactcgccgtcatgagcggcgcagaaagctagccatagagcgcgacaccatc

SEQ ID NO: 66 - protein, 21 aa, Synthetic construct
GNAKTRRHERRRKLAIERDTI

SEQ ID NO: 67 - DNA, 34 bp, Synthetic construct
  source 1..34: mol_type = other DNA
gatgtcatgtcatgagtccatggcatggcagtta

SEQ ID NO: 68 - DNA, 34 bp, Synthetic construct
  source 1..34: mol_type = other DNA
cgtgtcatgtcatgagtccatggcatggcatgat

SEQ ID NO: 69 - DNA, 34 bp, Synthetic construct
  source 1..34: mol_type = other DNA
attgacggtacggtacctgagtactgtactgtag

SEQ ID NO: 70 - DNA, 34 bp, Synthetic construct
  source 1..34: mol_type = other DNA
tagtacggtacgggacctgagtactgtactgtgc

SEQ ID NO: 71 - DNA, 1416 bp, Synthetic construct
  source 1..1416: mol_type = other DNA
atgggcagcaaggtggccggcaagaagaagacccagaacgacaacaagctcgacaacgagaacggcagccagcagagggagaacatcaacaccaagaccctcctcaagggcaacctcaagatcagcaactacaagtacctcgaggtgatccagctcgagcacgccgtgaccaagctcgtggagagctacaacaagatcatcgagctcagcccgaacctcgtggcctacaacgaggccgtgaacaaccaggacagggtgccggtgcagatcctcccgagcctcagcaggtatcagctcaagctcgccgccgagctcaagaccctccacgacctcaagaaggacgccatcctcaccgagatcaccgactacgagaacgagttcgacaccgagcagaagcagccgatcctccaggagatcagcaaggccgacatggagaagctcgagaagctcgagcaggtgaagagggagaagagggagaagatcgacgtgaacgtgtacgagaacctcaacgagaaggaggacgaggaggaggacgagggcgaggacagctacgacccgaccaaggccggcgacatcgtgaaggccaccaagtggccgccgaagctcccggagatccaggacctcgccatcagggccagggtgttcatccacaagagcaccatcaaggacaaggtgtacctcagcggcagcgagatgatcaacgcccacaacgagaggctcgagttcctcggcgacagcatcctcaacagcgtgatgaccctcatcatctacaacaagttcccggactacagcgagggccagctcagcaccctcaggatgaacctcgtgagcaacgagcagatcaagcagtggagcatcatgtacaacttccacgagaagctcaagaccaacttcgacctcaaggacgagaacagcaacttccagaacggcaagctcaagctctacgccgacgtgttcgaggcctacatcggcggcctcatggaggacgacccgaggaacaacctcccgaagatcaggaagtggctcaggaagctcgccaagccggtgatcgaggaggccaccaggaaccaggtggccctcgagaagaccgacaagctcgacatgaacgccaagaggcagctctacagcctcatcggctacgccagcctcaggctccactacgtgaccgtgaagaagccgaccgccgtggacccgaacagcatcgtggagtgcagggtgggcgacggcaccgtgctcggcaccggcgtgggcaggaacatcaagatcgccggcatcagggccgccgagaacgccctcagggacaagaagatgctcgacttctacgccaagcagagggccgccatcccgaggagcgagagcgtgctcaaggacccgagccagaagaacaagaagaggaagttcagcgacaccagctag

SEQ ID NO: 72 - protein, 471 aa, Saccharomyces cerevisiae; identical to SEQ ID NO: 45

SEQ ID NO: 73 - DNA, 1605 bp, Synthetic construct
  source 1..1605: mol_type = other DNA
atgggcagcaaggtggccggcaagaagaagacccagaacgacaacaagctcgacaacgagaacggcagccagcagagggagaacatcaacaccaagaccctcctcaagggcaacctcaagatcagcaactacaagtacctcgaggtgatccagctcgagcacgccgtgaccaagctcgtggagagctacaacaagatcatcgagctcagcccgaacctcgtggcctacaacgaggccgtgaacaaccaggacagggtgccggtgcagatcctcccgagcctcagcaggtatcagctcaagctcgccgccgagctcaagaccctccacgacctcaagaaggacgccatcctcaccgagatcaccgactacgagaacgagttcgacaccgagcagaagcagccgatcctccaggagatcagcaaggccgacatggagaagctcgagaagctcgagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtgaagagggagaagagggagaagatcgacgtgaacgtgtacgagaacctcaacgagaaggaggacgaggaggaggacgagggcgaggacagctacgacccgaccaaggccggcgacatcgtgaaggccaccaagtggccgccgaagctcccggagatccaggacctcgccatcagggccagggtgttcatccacaagagcaccatcaaggacaaggtgtacctcagcggcagcgagatgatcaacgcccacaacgagaggctcgagttcctcggcgacagcatcctcaacagcgtgatgaccctcatcatctacaacaagttcccggactacagcgagggccagctcagcaccctcaggatgaacctcgtgagcaacgagcagatcaagcagtggagcatcatgtacaacttccacgagaagctcaagaccaacttcgacctcaaggacgagaacagcaacttccagaacggcaagctcaagctctacgccgacgtgttcgaggcctacatcggcggcctcatggaggacgacccgaggaacaacctcccgaagatcaggaagtggctcaggaagctcgccaagccggtgatcgaggaggccaccaggaaccaggtggccctcgagaagaccgacaagctcgacatgaacgccaagaggcagctctacagcctcatcggctacgccagcctcaggctccactacgtgaccgtgaagaagccgaccgccgtggacccgaacagcatcgtggagtgcagggtgggcgacggcaccgtgctcggcaccggcgtgggcaggaacatcaagatcgccggcatcagggccgccgagaacgccctcagggacaagaagatgctcgacttctacgccaagcagagggccgccatcccgaggagcgagagcgtgctcaaggacccgagccagaagaacaagaagaggaagttcagcgacaccagctag

SEQ ID NO: 74 - DNA, 85 bp, Zea mays
  source 1..85: mol_type = unassigned DNA
gggtaatacgcaaacgttttgtcccaccttgactaatcacaagagtggagcgtaccttataaaccgagccgcaagcaccgaattg